SEQUENCE LISTING (891 sequences)

SEQ ID NO: 1 - RNA, 24 nt, Homo sapiens
  source 1..24: mol_type = tRNA
agccatgatcgtatagtggttagt

SEQ ID NO: 2 - RNA, 17 nt, Homo sapiens
  source 1..17: mol_type = tRNA
agccgtgatcgtatagt

SEQ ID NO: 3 - RNA, 18 nt, Homo sapiens
  source 1..18: mol_type = tRNA
agccgtgatcgtatagtg

SEQ ID NO: 4 - RNA, 19 nt, Homo sapiens
  source 1..19: mol_type = tRNA
agccgtgatcgtatagtgg

SEQ ID NO: 5 - RNA, 20 nt, Homo sapiens
  source 1..20: mol_type = tRNA
agccgtgatcgtatagtggt

SEQ ID NO: 6 - RNA, 21 nt, Homo sapiens
  source 1..21: mol_type = tRNA
agccgtgatcgtatagtggtt

SEQ ID NO: 7 - RNA, 22 nt, Homo sapiens
  source 1..22: mol_type = tRNA
agccgtgatcgtatagtggtta

SEQ ID NO: 8 - RNA, 23 nt, Homo sapiens
  source 1..23: mol_type = tRNA
agccgtgatcgtatagtggttag

SEQ ID NO: 9 - RNA, 24 nt, Homo sapiens
  source 1..24: mol_type = tRNA
agccgtgatcgtatagtggttagt

SEQ ID NO: 10 - RNA, 25 nt, Homo sapiens
  source 1..25: mol_type = tRNA
agccgtgatcgtatagtggttagta

SEQ ID NO: 11 - RNA, 26 nt, Homo sapiens
  source 1..26: mol_type = tRNA
agccgtgatcgtatagtggttagtac

SEQ ID NO: 12 - RNA, 27 nt, Homo sapiens
  source 1..27: mol_type = tRNA
agccgtgatcgtatagtggttagtact

SEQ ID NO: 13 - RNA, 28 nt, Homo sapiens
  source 1..28: mol_type = tRNA
agccgtgatcgtatagtggttagtactc

SEQ ID NO: 14 - RNA, 29 nt, Homo sapiens
  source 1..29: mol_type = tRNA
agccgtgatcgtatagtggttagtactct

SEQ ID NO: 15 - RNA, 30 nt, Homo sapiens
  source 1..30: mol_type = tRNA
agccgtgatcgtatagtggttagtactctg

SEQ ID NO: 16 - RNA, 19 nt, Homo sapiens
  source 1..19: mol_type = tRNA
cgccgtgatcgtatagtgg

SEQ ID NO: 17 - RNA, 21 nt, Homo sapiens
  source 1..21: mol_type = tRNA
cgccgtgatcgtatagtggtt

SEQ ID NO: 18 - RNA, 22 nt, Homo sapiens
  source 1..22: mol_type = tRNA
cgccgtgatcgtatagtggtta

SEQ ID NO: 19 - RNA, 23 nt, Homo sapiens
  source 1..23: mol_type = tRNA
cgccgtgatcgtatagtggttag

SEQ ID NO: 20 - RNA, 24 nt, Homo sapiens
  source 1..24: mol_type = tRNA
cgccgtgatcgtatagtggttagt

SEQ ID NO: 21 - RNA, 27 nt, Homo sapiens
  source 1..27: mol_type = tRNA
cgccgtgatcgtatagtggttagtact

SEQ ID NO: 22 - RNA, 28 nt, Homo sapiens
  source 1..28: mol_type = tRNA
cgccgtgatcgtatagtggttagtactc

SEQ ID NO: 23 - RNA, 29 nt, Homo sapiens
  source 1..29: mol_type = tRNA
cgccgtgatcgtatagtggttagtactct

SEQ ID NO: 24 - RNA, 30 nt, Homo sapiens
  source 1..30: mol_type = tRNA
cgccgtgatcgtatagtggttagtactctg

SEQ ID NO: 25 - RNA, 24 nt, Homo sapiens
  source 1..24: mol_type = tRNA
ggccatgatcgtatagtggttagt

SEQ ID NO: 26 - RNA, 16 nt, Homo sapiens
  source 1..16: mol_type = tRNA
ggccgtgatcgtatag

SEQ ID NO: 27 - RNA, 17 nt, Homo sapiens
  source 1..17: mol_type = tRNA
ggccgtgatcgtatagt

SEQ ID NO: 28 - RNA, 18 nt, Homo sapiens
  source 1..18: mol_type = tRNA
ggccgtgatcgtatagtg

SEQ ID NO: 29 - RNA, 19 nt, Homo sapiens
  source 1..19: mol_type = tRNA
ggccgtgatcgtatagtgg

SEQ ID NO: 30 - RNA, 20 nt, Homo sapiens
  source 1..20: mol_type = tRNA
ggccgtgatcgtatagtggt

SEQ ID NO: 31 - RNA, 21 nt, Homo sapiens
  source 1..21: mol_type = tRNA
ggccgtgatcgtatagtggtt

SEQ ID NO: 32 - RNA, 22 nt, Homo sapiens
  source 1..22: mol_type = tRNA
ggccgtgatcgtatagtggtta

SEQ ID NO: 33 - RNA, 23 nt, Homo sapiens
  source 1..23: mol_type = tRNA
ggccgtgatcgtatagtggttag

SEQ ID NO: 34 - RNA, 24 nt, Homo sapiens
  source 1..24: mol_type = tRNA
ggccgtgatcgtatagtggttagt

SEQ ID NO: 35 - RNA, 25 nt, Homo sapiens
  source 1..25: mol_type = tRNA
ggccgtgatcgtatagtggttagta

SEQ ID NO: 36 - RNA, 26 nt, Homo sapiens
  source 1..26: mol_type = tRNA
ggccgtgatcgtatagtggttagtac

SEQ ID NO: 37 - RNA, 27 nt, Homo sapiens
  source 1..27: mol_type = tRNA
ggccgtgatcgtatagtggttagtact

SEQ ID NO: 38 - RNA, 28 nt, Homo sapiens
  source 1..28: mol_type = tRNA
ggccgtgatcgtatagtggttagtactc

SEQ ID NO: 39 - RNA, 29 nt, Homo sapiens
  source 1..29: mol_type = tRNA
ggccgtgatcgtatagtggttagtactct

SEQ ID NO: 40 - RNA, 30 nt, Homo sapiens
  source 1..30: mol_type = tRNA
ggccgtgatcgtatagtggttagtactctg

SEQ ID NO: 41 - RNA, 17 nt, Homo sapiens
  source 1..17: mol_type = tRNA
ggtaaatatagtttaac

SEQ ID NO: 42 - RNA, 18 nt, Homo sapiens
  source 1..18: mol_type = tRNA
ggtaaatatagtttaacc

SEQ ID NO: 43 - RNA, 28 nt, Homo sapiens
  source 1..28: mol_type = tRNA
ggtaaatatagtttaaccaaaacatcag

SEQ ID NO: 44 - RNA, 19 nt, Homo sapiens
  source 1..19: mol_type = tRNA
tgccatgatcgtatagtgg

SEQ ID NO: 45 - RNA, 21 nt, Homo sapiens
  source 1..21: mol_type = tRNA
tgccatgatcgtatagtggtt

SEQ ID NO: 46 - RNA, 22 nt, Homo sapiens
  source 1..22: mol_type = tRNA
tgccatgatcgtatagtggtta

SEQ ID NO: 47 - RNA, 23 nt, Homo sapiens
  source 1..23: mol_type = tRNA
tgccatgatcgtatagtggttag

SEQ ID NO: 48 - RNA, 24 nt, Homo sapiens
  source 1..24: mol_type = tRNA
tgccatgatcgtatagtggttagt

SEQ ID NO: 49 - RNA, 25 nt, Homo sapiens
  source 1..25: mol_type = tRNA
tgccatgatcgtatagtggttagta

SEQ ID NO: 50 - RNA, 28 nt, Homo sapiens
  source 1..28: mol_type = tRNA
tgccatgatcgtatagtggttagtactc

SEQ ID NO: 51 - RNA, 30 nt, Homo sapiens
  source 1..30: mol_type = tRNA
tgccatgatcgtatagtggttagtactctg

SEQ ID NO: 52 - RNA, 16 nt, Homo sapiens
  source 1..16: mol_type = tRNA
tgccgtgatcgtatag

SEQ ID NO: 53 - RNA, 17 nt, Homo sapiens
  source 1..17: mol_type = tRNA
tgccgtgatcgtatagt

SEQ ID NO: 54 - RNA, 18 nt, Homo sapiens
  source 1..18: mol_type = tRNA
tgccgtgatcgtatagtg

SEQ ID NO: 55 - RNA, 19 nt, Homo sapiens
  source 1..19: mol_type = tRNA
tgccgtgatcgtatagtgg

SEQ ID NO: 56 - RNA, 20 nt, Homo sapiens
  source 1..20: mol_type = tRNA
tgccgtgatcgtatagtggt

SEQ ID NO: 57 - RNA, 21 nt, Homo sapiens
  source 1..21: mol_type = tRNA
tgccgtgatcgtatagtggtt

SEQ ID NO: 58 - RNA, 22 nt, Homo sapiens
  source 1..22: mol_type = tRNA
tgccgtgatcgtatagtggtta

SEQ ID NO: 59 - RNA, 23 nt, Homo sapiens
  source 1..23: mol_type = tRNA
tgccgtgatcgtatagtggttag

SEQ ID NO: 60 - RNA, 24 nt, Homo sapiens
  source 1..24: mol_type = tRNA
tgccgtgatcgtatagtggttagt

SEQ ID NO: 61 - RNA, 25 nt, Homo sapiens
  source 1..25: mol_type = tRNA
tgccgtgatcgtatagtggttagta

SEQ ID NO: 62 - RNA, 26 nt, Homo sapiens
  source 1..26: mol_type = tRNA
tgccgtgatcgtatagtggttagtac

SEQ ID NO: 63 - RNA, 27 nt, Homo sapiens
  source 1..27: mol_type = tRNA
tgccgtgatcgtatagtggttagtact

SEQ ID NO: 64 - RNA, 28 nt, Homo sapiens
  source 1..28: mol_type = tRNA
tgccgtgatcgtatagtggttagtactc

SEQ ID NO: 65 - RNA, 29 nt, Homo sapiens
  source 1..29: mol_type = tRNA
tgccgtgatcgtatagtggttagtactct

SEQ ID NO: 66 - RNA, 30 nt, Homo sapiens
  source 1..30: mol_type = tRNA
tgccgtgatcgtatagtggttagtactctg

SEQ ID NO: 67 - RNA, 27 nt, Homo sapiens
  source 1..27: mol_type = tRNA
gccatgatcgtatagtggttagtactc

SEQ ID NO: 68 - RNA, 28 nt, Homo sapiens
  source 1..28: mol_type = tRNA
gccatgatcgtatagtggttagtactct

SEQ ID NO: 69 - RNA, 29 nt, Homo sapiens
  source 1..29: mol_type = tRNA
gccatgatcgtatagtggttagtactctg

SEQ ID NO: 70 - RNA, 30 nt, Homo sapiens
  source 1..30: mol_type = tRNA
gccatgatcgtatagtggttagtactctgc

SEQ ID NO: 71 - RNA, 16 nt, Homo sapiens
  source 1..16: mol_type = tRNA
gccgtgatcgtatagt

SEQ ID NO: 72 - RNA, 17 nt, Homo sapiens
  source 1..17: mol_type = tRNA
gccgtgatcgtatagtg

SEQ ID NO: 73 - RNA, 18 nt, Homo sapiens
  source 1..18: mol_type = tRNA
gccgtgatcgtatagtgg

SEQ ID NO: 74 - RNA, 19 nt, Homo sapiens
  source 1..19: mol_type = tRNA
gccgtgatcgtatagtggt

SEQ ID NO: 75 - RNA, 20 nt, Homo sapiens
  source 1..20: mol_type = tRNA
gccgtgatcgtatagtggtt

SEQ ID NO: 76 - RNA, 21 nt, Homo sapiens
  source 1..21: mol_type = tRNA
gccgtgatcgtatagtggtta

SEQ ID NO: 77 - RNA, 22 nt, Homo sapiens
  source 1..22: mol_type = tRNA
gccgtgatcgtatagtggttag

SEQ ID NO: 78 - RNA, 23 nt, Homo sapiens
  source 1..23: mol_type = tRNA
gccgtgatcgtatagtggttagt

SEQ ID NO: 79 - RNA, 24 nt, Homo sapiens
  source 1..24: mol_type = tRNA
gccgtgatcgtatagtggttagta

SEQ ID NO: 80 - RNA, 25 nt, Homo sapiens
  source 1..25: mol_type = tRNA
gccgtgatcgtatagtggttagtac

SEQ ID NO: 81 - RNA, 26 nt, Homo sapiens
  source 1..26: mol_type = tRNA
gccgtgatcgtatagtggttagtact

SEQ ID NO: 82 - RNA, 27 nt, Homo sapiens
  source 1..27: mol_type = tRNA
gccgtgatcgtatagtggttagtactc

SEQ ID NO: 83 - RNA, 28 nt, Homo sapiens
  source 1..28: mol_type = tRNA
gccgtgatcgtatagtggttagtactct

SEQ ID NO: 84 - RNA, 29 nt, Homo sapiens
  source 1..29: mol_type = tRNA
gccgtgatcgtatagtggttagtactctg

SEQ ID NO: 85 - RNA, 30 nt, Homo sapiens
  source 1..30: mol_type = tRNA
gccgtgatcgtatagtggttagtactctgc

SEQ ID NO: 86 - RNA, 17 nt, Homo sapiens
  source 1..17: mol_type = tRNA
gtaaatatagtttaacc

SEQ ID NO: 87 - RNA, 30 nt, Homo sapiens
  source 1..30: mol_type = tRNA
gtaaatatagtttaaccaaaacatcagatt

SEQ ID NO: 88 - RNA, 16 nt, Homo sapiens
  source 1..16: mol_type = tRNA
aaaacatcagattgtg

SEQ ID NO: 89 - RNA, 17 nt, Homo sapiens
  source 1..17: mol_type = tRNA
aaaacatcagattgtga

SEQ ID NO: 90 - RNA, 18 nt, Homo sapiens
  source 1..18: mol_type = tRNA
aaaacatcagattgtgaa

SEQ ID NO: 91 - RNA, 19 nt, Homo sapiens
  source 1..19: mol_type = tRNA
aaaacatcagattgtgaat

SEQ ID NO: 92 - RNA, 20 nt, Homo sapiens
  source 1..20: mol_type = tRNA
aaaacatcagattgtgaatc

SEQ ID NO: 93 - RNA, 21 nt, Homo sapiens
  source 1..21: mol_type = tRNA
aaaacatcagattgtgaatct

SEQ ID NO: 94 - RNA, 22 nt, Homo sapiens
  source 1..22: mol_type = tRNA
aaaacatcagattgtgaatctg

SEQ ID NO: 95 - RNA, 23 nt, Homo sapiens
  source 1..23: mol_type = tRNA
aaaacatcagattgtgaatctga

SEQ ID NO: 96 - RNA, 24 nt, Homo sapiens
  source 1..24: mol_type = tRNA
aaaacatcagattgtgaatctgac

SEQ ID NO: 97 - RNA, 25 nt, Homo sapiens
  source 1..25: mol_type = tRNA
aaaacatcagattgtgaatctgaca

SEQ ID NO: 98 - RNA, 26 nt, Homo sapiens
  source 1..26: mol_type = tRNA
aaaacatcagattgtgaatctgacaa

SEQ ID NO: 99 - RNA, 27 nt, Homo sapiens
  source 1..27: mol_type = tRNA
aaaacatcagattgtgaatctgacaac

SEQ ID NO: 100 - RNA, 28 nt, Homo sapiens
  source 1..28: mol_type = tRNA
aaaacatcagattgtgaatctgacaaca

SEQ ID NO: 101 - RNA, 29 nt, Homo sapiens
  source 1..29: mol_type = tRNA
aaaacatcagattgtgaatctgacaacag

SEQ ID NO: 102 - RNA, 16 nt, Homo sapiens
  source 1..16: mol_type = tRNA
aaacatcagattgtga

SEQ ID NO: 103 - RNA, 18 nt, Homo sapiens
  source 1..18: mol_type = tRNA
aaacatcagattgtgaat

SEQ ID NO: 104 - RNA, 19 nt, Homo sapiens
  source 1..19: mol_type = tRNA
aaacatcagattgtgaatc

SEQ ID NO: 105 - RNA, 20 nt, Homo sapiens
  source 1..20: mol_type = tRNA
aaacatcagattgtgaatct

SEQ ID NO: 106 - RNA, 21 nt, Homo sapiens
  source 1..21: mol_type = tRNA
aaacatcagattgtgaatctg

SEQ ID NO: 107 - RNA, 22 nt, Homo sapiens
  source 1..22: mol_type = tRNA
aaacatcagattgtgaatctga

SEQ ID NO: 108 - RNA, 23 nt, Homo sapiens
  source 1..23: mol_type = tRNA
aaacatcagattgtgaatctgac

SEQ ID NO: 109 - RNA, 24 nt, Homo sapiens
  source 1..24: mol_type = tRNA
aaacatcagattgtgaatctgaca

SEQ ID NO: 110 - RNA, 25 nt, Homo sapiens
  source 1..25: mol_type = tRNA
aaacatcagattgtgaatctgacaa

SEQ ID NO: 111 - RNA, 26 nt, Homo sapiens
  source 1..26: mol_type = tRNA
aaacatcagattgtgaatctgacaac

SEQ ID NO: 112 - RNA, 27 nt, Homo sapiens
  source 1..27: mol_type = tRNA
aaacatcagattgtgaatctgacaaca

SEQ ID NO: 113 - RNA, 28 nt, Homo sapiens
  source 1..28: mol_type = tRNA
aaacatcagattgtgaatctgacaacag

SEQ ID NO: 114 - RNA, 29 nt, Homo sapiens
  source 1..29: mol_type = tRNA
aaacatcagattgtgaatctgacaacaga

SEQ ID NO: 115 - RNA, 30 nt, Homo sapiens
  source 1..30: mol_type = tRNA
aaacatcagattgtgaatctgacaacagag

SEQ ID NO: 116 - RNA, 19 nt, Homo sapiens
  source 1..19: mol_type = tRNA
aacagaggcttacgacccc

SEQ ID NO: 117 - RNA, 17 nt, Homo sapiens
  source 1..17: mol_type = tRNA
aacatcagattgtgaat

SEQ ID NO: 118 - RNA, 18 nt, Homo sapiens
  source 1..18: mol_type = tRNA
aacatcagattgtgaatc

SEQ ID NO: 119 - RNA, 19 nt, Homo sapiens
  source 1..19: mol_type = tRNA
aacatcagattgtgaatct

SEQ ID NO: 120 - RNA, 20 nt, Homo sapiens
  source 1..20: mol_type = tRNA
aacatcagattgtgaatctg

SEQ ID NO: 121 - RNA, 21 nt, Homo sapiens
  source 1..21: mol_type = tRNA
aacatcagattgtgaatctga

SEQ ID NO: 122 - RNA, 22 nt, Homo sapiens
  source 1..22: mol_type = tRNA
aacatcagattgtgaatctgac

SEQ ID NO: 123 - RNA, 23 nt, Homo sapiens
  source 1..23: mol_type = tRNA
aacatcagattgtgaatctgaca

SEQ ID NO: 124 - RNA, 24 nt, Homo sapiens
  source 1..24: mol_type = tRNA
aacatcagattgtgaatctgacaa

SEQ ID NO: 125 - RNA, 25 nt, Homo sapiens
  source 1..25: mol_type = tRNA
aacatcagattgtgaatctgacaac

SEQ ID NO: 126 - RNA, 26 nt, Homo sapiens
  source 1..26: mol_type = tRNA
aacatcagattgtgaatctgacaaca

SEQ ID NO: 127 - RNA, 27 nt, Homo sapiens
  source 1..27: mol_type = tRNA
aacatcagattgtgaatctgacaacag

SEQ ID NO: 128 - RNA, 28 nt, Homo sapiens
  source 1..28: mol_type = tRNA
aacatcagattgtgaatctgacaacaga

SEQ ID NO: 129 - RNA, 29 nt, Homo sapiens
  source 1..29: mol_type = tRNA
aacatcagattgtgaatctgacaacagag

SEQ ID NO: 130 - RNA, 30 nt, Homo sapiens
  source 1..30: mol_type = tRNA
aacatcagattgtgaatctgacaacagagg

SEQ ID NO: 131 - RNA, 18 nt, Homo sapiens
  source 1..18: mol_type = tRNA
aaccaaaacatcagattg

SEQ ID NO: 132 - RNA, 19 nt, Homo sapiens
  source 1..19: mol_type = tRNA
aaccaaaacatcagattgt

SEQ ID NO: 133 - RNA, 20 nt, Homo sapiens
  source 1..20: mol_type = tRNA
aaccaaaacatcagattgtg

SEQ ID NO: 134 - RNA, 21 nt, Homo sapiens
  source 1..21: mol_type = tRNA
aaccaaaacatcagattgtga

SEQ ID NO: 135 - RNA, 23 nt, Homo sapiens
  source 1..23: mol_type = tRNA
aaccaaaacatcagattgtgaat

SEQ ID NO: 136 - RNA, 24 nt, Homo sapiens
  source 1..24: mol_type = tRNA
aaccaaaacatcagattgtgaatc

SEQ ID NO: 137 - RNA, 25 nt, Homo sapiens
  source 1..25: mol_type = tRNA
aaccaaaacatcagattgtgaatct

SEQ ID NO: 138 - RNA, 29 nt, Homo sapiens
  source 1..29: mol_type = tRNA
aaccaaaacatcagattgtgaatctgaca

SEQ ID NO: 139 - RNA, 25 nt, Homo sapiens
  source 1..25: mol_type = tRNA
aacctcggttcgaatccgagtcacg

SEQ ID NO: 140 - RNA, 18 nt, Homo sapiens
  source 1..18: mol_type = tRNA
aatctgacaacagaggct

SEQ ID NO: 141 - RNA, 19 nt, Homo sapiens
  source 1..19: mol_type = tRNA
aatctgacaacagaggctt

SEQ ID NO: 142 - RNA, 20 nt, Homo sapiens
  source 1..20: mol_type = tRNA
aatctgacaacagaggctta

SEQ ID NO: 143 - RNA, 22 nt, Homo sapiens
  source 1..22: mol_type = tRNA
aatctgacaacagaggcttacg

SEQ ID NO: 144 - RNA, 29 nt, Homo sapiens
  source 1..29: mol_type = tRNA
aatctgacaacagaggcttacgacccctt

SEQ ID NO: 145 - RNA, 30 nt, Homo sapiens
  source 1..30: mol_type = tRNA
acaacagaggcttacgaccccttatttacc

SEQ ID NO: 146 - RNA, 18 nt, Homo sapiens
  source 1..18: mol_type = tRNA
acagaggcttacgacccc

SEQ ID NO: 147 - RNA, 19 nt, Homo sapiens
  source 1..19: mol_type = tRNA
acagaggcttacgacccct

SEQ ID NO: 148 - RNA, 20 nt, Homo sapiens
  source 1..20: mol_type = tRNA
acagaggcttacgacccctt

SEQ ID NO: 149 - RNA, 21 nt, Homo sapiens
  source 1..21: mol_type = tRNA
acagaggcttacgacccctta

SEQ ID NO: 150 - RNA, 23 nt, Homo sapiens
  source 1..23: mol_type = tRNA
acagaggcttacgaccccttatt

SEQ ID NO: 151 - RNA, 27 nt, Homo sapiens
  source 1..27: mol_type = tRNA
acagaggcttacgaccccttatttacc

SEQ ID NO: 152 - RNA, 16 nt, Homo sapiens
  source 1..16: mol_type = tRNA
acatcagattgtgaat

SEQ ID NO: 153 - RNA, 17 nt, Homo sapiens
  source 1..17: mol_type = tRNA
acatcagattgtgaatc

SEQ ID NO: 154 - RNA, 18 nt, Homo sapiens
  source 1..18: mol_type = tRNA
acatcagattgtgaatct

SEQ ID NO: 155 - RNA, 20 nt, Homo sapiens
  source 1..20: mol_type = tRNA
acatcagattgtgaatctga

SEQ ID NO: 156 - RNA, 21 nt, Homo sapiens
  source 1..21: mol_type = tRNA
acatcagattgtgaatctgac

SEQ ID NO: 157 - RNA, 22 nt, Homo sapiens
  source 1..22: mol_type = tRNA
acatcagattgtgaatctgaca

SEQ ID NO: 158 - RNA, 23 nt, Homo sapiens
  source 1..23: mol_type = tRNA
acatcagattgtgaatctgacaa

SEQ ID NO: 159 - RNA, 24 nt, Homo sapiens
  source 1..24: mol_type = tRNA
acatcagattgtgaatctgacaac

SEQ ID NO: 160 - RNA, 25 nt, Homo sapiens
  source 1..25: mol_type = tRNA
acatcagattgtgaatctgacaaca

SEQ ID NO: 161 - RNA, 26 nt, Homo sapiens
  source 1..26: mol_type = tRNA
acatcagattgtgaatctgacaacag

SEQ ID NO: 162 - RNA, 27 nt, Homo sapiens
  source 1..27: mol_type = tRNA
acatcagattgtgaatctgacaacaga

SEQ ID NO: 163 - RNA, 28 nt, Homo sapiens
  source 1..28: mol_type = tRNA
acatcagattgtgaatctgacaacagag

SEQ ID NO: 164 - RNA, 29 nt, Homo sapiens
  source 1..29: mol_type = tRNA
acatcagattgtgaatctgacaacagagg

SEQ ID NO: 165 - RNA, 30 nt, Homo sapiens
  source 1..30: mol_type = tRNA
acatcagattgtgaatctgacaacagaggc

SEQ ID NO: 166 - RNA, 18 nt, Homo sapiens
  source 1..18: mol_type = tRNA
accaaaacatcagattgt

SEQ ID NO: 167 - RNA, 19 nt, Homo sapiens
  source 1..19: mol_type = tRNA
accaaaacatcagattgtg

SEQ ID NO: 168 - RNA, 20 nt, Homo sapiens
  source 1..20: mol_type = tRNA
accaaaacatcagattgtga

SEQ ID NO: 169 - RNA, 23 nt, Homo sapiens
  source 1..23: mol_type = tRNA
accaaaacatcagattgtgaatc

SEQ ID NO: 170 - RNA, 24 nt, Homo sapiens
  source 1..24: mol_type = tRNA
accaaaacatcagattgtgaatct

SEQ ID NO: 171 - RNA, 28 nt, Homo sapiens
  source 1..28: mol_type = tRNA
accaaaacatcagattgtgaatctgaca

SEQ ID NO: 172 - RNA, 30 nt, Homo sapiens
  source 1..30: mol_type = tRNA
accaaaacatcagattgtgaatctgacaac

SEQ ID NO: 173 - RNA, 16 nt, Homo sapiens
  source 1..16: mol_type = tRNA
actctgcgttgtggcc

SEQ ID NO: 174 - RNA, 17 nt, Homo sapiens
  source 1..17: mol_type = tRNA
actctgcgttgtggccg

SEQ ID NO: 175 - RNA, 18 nt, Homo sapiens
  source 1..18: mol_type = tRNA
actctgcgttgtggccgc

SEQ ID NO: 176 - RNA, 19 nt, Homo sapiens
  source 1..19: mol_type = tRNA
actctgcgttgtggccgca

SEQ ID NO: 177 - RNA, 21 nt, Homo sapiens
  source 1..21: mol_type = tRNA
actctgcgttgtggccgcagc

SEQ ID NO: 178 - RNA, 25 nt, Homo sapiens
  source 1..25: mol_type = tRNA
actctgcgttgtggccgcagcaacc

SEQ ID NO: 179 - RNA, 30 nt, Homo sapiens
  source 1..30: mol_type = tRNA
actctgcgttgtggccgcagcaacctcggt

SEQ ID NO: 180 - RNA, 16 nt, Homo sapiens
  source 1..16: mol_type = tRNA
agaggcttacgacccc

SEQ ID NO: 181 - RNA, 25 nt, Homo sapiens
  source 1..25: mol_type = tRNA
agaggcttacgaccccttatttacc

SEQ ID NO: 182 - RNA, 16 nt, Homo sapiens
  source 1..16: mol_type = tRNA
agattgtgaatctgac

SEQ ID NO: 183 - RNA, 17 nt, Homo sapiens
  source 1..17: mol_type = tRNA
agattgtgaatctgaca

SEQ ID NO: 184 - RNA, 18 nt, Homo sapiens
  source 1..18: mol_type = tRNA
agattgtgaatctgacaa

SEQ ID NO: 185 - RNA, 19 nt, Homo sapiens
  source 1..19: mol_type = tRNA
agattgtgaatctgacaac

SEQ ID NO: 186 - RNA, 20 nt, Homo sapiens
  source 1..20: mol_type = tRNA
agattgtgaatctgacaaca

SEQ ID NO: 187 - RNA, 21 nt, Homo sapiens
  source 1..21: mol_type = tRNA
agattgtgaatctgacaacag

SEQ ID NO: 188 - RNA, 22 nt, Homo sapiens
  source 1..22: mol_type = tRNA
agattgtgaatctgacaacaga

SEQ ID NO: 189 - RNA, 23 nt, Homo sapiens
  source 1..23: mol_type = tRNA
agattgtgaatctgacaacagag

SEQ ID NO: 190 - RNA, 24 nt, Homo sapiens
  source 1..24: mol_type = tRNA
agattgtgaatctgacaacagagg

SEQ ID NO: 191 - RNA, 25 nt, Homo sapiens
  source 1..25: mol_type = tRNA
agattgtgaatctgacaacagaggc

SEQ ID NO: 192 - RNA, 26 nt, Homo sapiens
  source 1..26: mol_type = tRNA
agattgtgaatctgacaacagaggct

SEQ ID NO: 193 - RNA, 27 nt, Homo sapiens
  source 1..27: mol_type = tRNA
agattgtgaatctgacaacagaggctt

SEQ ID NO: 194 - RNA, 29 nt, Homo sapiens
  source 1..29: mol_type = tRNA
agattgtgaatctgacaacagaggcttac

SEQ ID NO: 195 - RNA, 30 nt, Homo sapiens
  source 1..30: mol_type = tRNA
agattgtgaatctgacaacagaggcttacg

SEQ ID NO: 196 - RNA, 23 nt, Homo sapiens
  source 1..23: mol_type = tRNA
aggcttacgaccccttatttacc

SEQ ID NO: 197 - RNA, 16 nt, Homo sapiens
  source 1..16: mol_type = tRNA
agtactctgcgttgtg

SEQ ID NO: 198 - RNA, 17 nt, Homo sapiens
  source 1..17: mol_type = tRNA
agtactctgcgttgtgg

SEQ ID NO: 199 - RNA, 18 nt, Homo sapiens
  source 1..18: mol_type = tRNA
agtactctgcgttgtggc

SEQ ID NO: 200 - RNA, 19 nt, Homo sapiens
  source 1..19: mol_type = tRNA
agtactctgcgttgtggcc

SEQ ID NO: 201 - RNA, 20 nt, Homo sapiens
  source 1..20: mol_type = tRNA
agtactctgcgttgtggccg

SEQ ID NO: 202 - RNA, 21 nt, Homo sapiens
  source 1..21: mol_type = tRNA
agtactctgcgttgtggccgc

SEQ ID NO: 203 - RNA, 22 nt, Homo sapiens
  source 1..22: mol_type = tRNA
agtactctgcgttgtggccgca

SEQ ID NO: 204 - RNA, 30 nt, Homo sapiens
  source 1..30: mol_type = tRNA
agtactctgcgttgtggccgcagcaacctc

SEQ ID NO: 205 - RNA, 16 nt, Homo sapiens
  source 1..16: mol_type = tRNA
agtggttagtactctg

SEQ ID NO: 206 - RNA, 17 nt, Homo sapiens
  source 1..17: mol_type = tRNA
agtggttagtactctgc

SEQ ID NO: 207 - RNA, 18 nt, Homo sapiens
  source 1..18: mol_type = tRNA
agtggttagtactctgcg

SEQ ID NO: 208 - RNA, 19 nt, Homo sapiens
  source 1..19: mol_type = tRNA
agtggttagtactctgcgc

SEQ ID NO: 209 - RNA, 20 nt, Homo sapiens
  source 1..20: mol_type = tRNA
agtggttagtactctgcgct

SEQ ID NO: 210 - RNA, 19 nt, Homo sapiens
  source 1..19: mol_type = tRNA
agtggttagtactctgcgt

SEQ ID NO: 211 - RNA, 20 nt, Homo sapiens
  source 1..20: mol_type = tRNA
agtggttagtactctgcgtt

SEQ ID NO: 212 - RNA, 21 nt, Homo sapiens
  source 1..21: mol_type = tRNA
agtggttagtactctgcgttg

SEQ ID NO: 213 - RNA, 22 nt, Homo sapiens
  source 1..22: mol_type = tRNA
agtggttagtactctgcgttgt

SEQ ID NO: 214 - RNA, 23 nt, Homo sapiens
  source 1..23: mol_type = tRNA
agtggttagtactctgcgttgtg

SEQ ID NO: 215 - RNA, 24 nt, Homo sapiens
  source 1..24: mol_type = tRNA
agtggttagtactctgcgttgtgg

SEQ ID NO: 216 - RNA, 25 nt, Homo sapiens
  source 1..25: mol_type = tRNA
agtggttagtactctgcgttgtggc

SEQ ID NO: 217 - RNA, 26 nt, Homo sapiens
  source 1..26: mol_type = tRNA
agtggttagtactctgcgttgtggcc

SEQ ID NO: 218 - RNA, 27 nt, Homo sapiens
  source 1..27: mol_type = tRNA
agtggttagtactctgcgttgtggccg

SEQ ID NO: 219 - RNA, 19 nt, Homo sapiens
  source 1..19: mol_type = tRNA
agtttaaccaaaacatcag

SEQ ID NO: 220 - RNA, 25 nt, Homo sapiens
  source 1..25: mol_type = tRNA
agtttaaccaaaacatcagattgtg

SEQ ID NO: 221 - RNA, 26 nt, Homo sapiens
  source 1..26: mol_type = tRNA
agtttaaccaaaacatcagattgtga

SEQ ID NO: 222 - RNA, 29 nt, Homo sapiens
  source 1..29: mol_type = tRNA
agtttaaccaaaacatcagattgtgaatc

SEQ ID NO: 223 - RNA, 17 nt, Homo sapiens
  source 1..17: mol_type = tRNA
atagtggttagtactct

SEQ ID NO: 224 - RNA, 18 nt, Homo sapiens
  source 1..18: mol_type = tRNA
atagtggttagtactctg

SEQ ID NO: 225 - RNA, 19 nt, Homo sapiens
  source 1..19: mol_type = tRNA
atagtggttagtactctgc

SEQ ID NO: 226 - RNA, 20 nt, Homo sapiens
  source 1..20: mol_type = tRNA
atagtggttagtactctgcg

SEQ ID NO: 227 - RNA, 23 nt, Homo sapiens
  source 1..23: mol_type = tRNA
atagtggttagtactctgcgctg

SEQ ID NO: 228 - RNA, 21 nt, Homo sapiens
  source 1..21: mol_type = tRNA
atagtggttagtactctgcgt

SEQ ID NO: 229 - RNA, 22 nt, Homo sapiens
  source 1..22: mol_type = tRNA
atagtggttagtactctgcgtt

SEQ ID NO: 230 - RNA, 23 nt, Homo sapiens
  source 1..23: mol_type = tRNA
atagtggttagtactctgcgttg

SEQ ID NO: 231 - RNA, 24 nt, Homo sapiens
  source 1..24: mol_type = tRNA
atagtggttagtactctgcgttgt

SEQ ID NO: 232 - RNA, 25 nt, Homo sapiens
  source 1..25: mol_type = tRNA
atagtggttagtactctgcgttgtg

SEQ ID NO: 233 - RNA, 26 nt, Homo sapiens
  source 1..26: mol_type = tRNA
atagtggttagtactctgcgttgtgg

SEQ ID NO: 234 - RNA, 27 nt, Homo sapiens
  source 1..27: mol_type = tRNA
atagtggttagtactctgcgttgtggc

SEQ ID NO: 235 - RNA, 28 nt, Homo sapiens
  source 1..28: mol_type = tRNA
atagtggttagtactctgcgttgtggcc

SEQ ID NO: 236 - RNA, 29 nt, Homo sapiens
  source 1..29: mol_type = tRNA
atagtggttagtactctgcgttgtggccg

SEQ ID NO: 237 - RNA, 28 nt, Homo sapiens
  source 1..28: mol_type = tRNA
atagtttaaccaaaacatcagattgtga

SEQ ID NO: 238 - RNA, 29 nt, Homo sapiens
  source 1..29: mol_type = tRNA
atatagtttaaccaaaacatcagattgtg

SEQ ID NO: 239 - RNA, 18 nt, Homo sapiens
  source 1..18: mol_type = tRNA
atcagattgtgaatctga

SEQ ID NO: 240 - RNA, 19 nt, Homo sapiens
  source 1..19: mol_type = tRNA
atcagattgtgaatctgac

SEQ ID NO: 241 - RNA, 20 nt, Homo sapiens
  source 1..20: mol_type = tRNA
atcagattgtgaatctgaca

SEQ ID NO: 242 - RNA, 23 nt, Homo sapiens
  source 1..23: mol_type = tRNA
atcagattgtgaatctgacaaca

SEQ ID NO: 243 - RNA, 24 nt, Homo sapiens
  source 1..24: mol_type = tRNA
atcagattgtgaatctgacaacag

SEQ ID NO: 244 - RNA, 27 nt, Homo sapiens
  source 1..27: mol_type = tRNA
atcagattgtgaatctgacaacagagg

SEQ ID NO: 245 - RNA, 29 nt, Homo sapiens
  source 1..29: mol_type = tRNA
atcagattgtgaatctgacaacagaggct

SEQ ID NO: 246 - RNA, 17 nt, Homo sapiens
  source 1..17: mol_type = tRNA
atcgtatagtggttagt

SEQ ID NO: 247 - RNA, 18 nt, Homo sapiens
  source 1..18: mol_type = tRNA
atcgtatagtggttagta

SEQ ID NO: 248 - RNA, 20 nt, Homo sapiens
  source 1..20: mol_type = tRNA
atcgtatagtggttagtact

SEQ ID NO: 249 - RNA, 21 nt, Homo sapiens
  source 1..21: mol_type = tRNA
atcgtatagtggttagtactc

SEQ ID NO: 250 - RNA, 22 nt, Homo sapiens
  source 1..22: mol_type = tRNA
atcgtatagtggttagtactct

SEQ ID NO: 251 - RNA, 23 nt, Homo sapiens
  source 1..23: mol_type = tRNA
atcgtatagtggttagtactctg

SEQ ID NO: 252 - RNA, 24 nt, Homo sapiens
  source 1..24: mol_type = tRNA
atcgtatagtggttagtactctgc

SEQ ID NO: 253 - RNA, 25 nt, Homo sapiens
  source 1..25: mol_type = tRNA
atcgtatagtggttagtactctgcg

SEQ ID NO: 254 - RNA, 26 nt, Homo sapiens
  source 1..26: mol_type = tRNA
atcgtatagtggttagtactctgcgt

SEQ ID NO: 255 - RNA, 27 nt, Homo sapiens
  source 1..27: mol_type = tRNA
atcgtatagtggttagtactctgcgtt

SEQ ID NO: 256 - RNA, 28 nt, Homo sapiens
  source 1..28: mol_type = tRNA
atcgtatagtggttagtactctgcgttg

SEQ ID NO: 257 - RNA, 29 nt, Homo sapiens
  source 1..29: mol_type = tRNA
atcgtatagtggttagtactctgcgttgt

SEQ ID NO: 258 - RNA, 30 nt, Homo sapiens
  source 1..30: mol_type = tRNA
atcgtatagtggttagtactctgcgttgtg

SEQ ID NO: 259 - RNA, 28 nt, Homo sapiens
  source 1..28: mol_type = tRNA
atctgacaacagaggcttacgacccctt

SEQ ID NO: 260 - RNA, 19 nt, Homo sapiens
  source 1..19: mol_type = tRNA
atgatcgtatagtggttag

SEQ ID NO: 261 - RNA, 20 nt, Homo sapiens
  source 1..20: mol_type = tRNA
atgatcgtatagtggttagt

SEQ ID NO: 262 - RNA, 21 nt, Homo sapiens
  source 1..21: mol_type = tRNA
atgatcgtatagtggttagta

SEQ ID NO: 263 - RNA, 23 nt, Homo sapiens
  source 1..23: mol_type = tRNA
atgatcgtatagtggttagtact

SEQ ID NO: 264 - RNA, 24 nt, Homo sapiens
  source 1..24: mol_type = tRNA
atgatcgtatagtggttagtactc

SEQ ID NO: 265 - RNA, 25 nt, Homo sapiens
  source 1..25: mol_type = tRNA
atgatcgtatagtggttagtactct

SEQ ID NO: 266 - RNA, 26 nt, Homo sapiens
  source 1..26: mol_type = tRNA
atgatcgtatagtggttagtactctg

SEQ ID NO: 267 - RNA, 27 nt, Homo sapiens
  source 1..27: mol_type = tRNA
atgatcgtatagtggttagtactctgc

SEQ ID NO: 268 - RNA, 28 nt, Homo sapiens
  source 1..28: mol_type = tRNA
atgatcgtatagtggttagtactctgcg

SEQ ID NO: 269 - RNA, 16 nt, Homo sapiens
  source 1..16: mol_type = tRNA
attgtgaatctgacaa

SEQ ID NO: 270 - RNA, 17 nt, Homo sapiens
  source 1..17: mol_type = tRNA
attgtgaatctgacaac

SEQ ID NO: 271 - RNA, 18 nt, Homo sapiens
  source 1..18: mol_type = tRNA
attgtgaatctgacaaca

SEQ ID NO: 272 - RNA, 19 nt, Homo sapiens
  source 1..19: mol_type = tRNA
attgtgaatctgacaacag

SEQ ID NO: 273 - RNA, 20 nt, Homo sapiens
  source 1..20: mol_type = tRNA
attgtgaatctgacaacaga

SEQ ID NO: 274 - RNA, 21 nt, Homo sapiens
  source 1..21: mol_type = tRNA
attgtgaatctgacaacagag

SEQ ID NO: 275 - RNA, 22 nt, Homo sapiens
  source 1..22: mol_type = tRNA
attgtgaatctgacaacagagg

SEQ ID NO: 276 - RNA, 23 nt, Homo sapiens
  source 1..23: mol_type = tRNA
attgtgaatctgacaacagaggc

SEQ ID NO: 277 - RNA, 24 nt, Homo sapiens
  source 1..24: mol_type = tRNA
attgtgaatctgacaacagaggct

SEQ ID NO: 278 - RNA, 25 nt, Homo sapiens
  source 1..25: mol_type = tRNA
attgtgaatctgacaacagaggctt

SEQ ID NO: 279 - RNA, 26 nt, Homo sapiens
  source 1..26: mol_type = tRNA
attgtgaatctgacaacagaggctta

SEQ ID NO: 280 - RNA, 27 nt, Homo sapiens
  source 1..27: mol_type = tRNA
attgtgaatctgacaacagaggcttac

SEQ ID NO: 281 - RNA, 28 nt, Homo sapiens
  source 1..28: mol_type = tRNA
attgtgaatctgacaacagaggcttacg

SEQ ID NO: 282 - RNA, 29 nt, Homo sapiens
  source 1..29: mol_type = tRNA
attgtgaatctgacaacagaggcttacga

SEQ ID NO: 283 - RNA, 30 nt, Homo sapiens
  source 1..30: mol_type = tRNA
attgtgaatctgacaacagaggcttacgac

SEQ ID NO: 284 - RNA, 17 nt, Homo sapiens
  source 1..17: mol_type = tRNA
caaaacatcagattgtg

SEQ ID NO: 285 - RNA, 18 nt, Homo sapiens
  source 1..18: mol_type = tRNA
caaaacatcagattgtga

SEQ ID NO: 286 - RNA, 19 nt, Homo sapiens
  source 1..19: mol_type = tRNA
caaaacatcagattgtgaa

SEQ ID NO: 287 - RNA, 20 nt, Homo sapiens
  source 1..20: mol_type = tRNA
caaaacatcagattgtgaat

SEQ ID NO: 288 - RNA, 21 nt, Homo sapiens
  source 1..21: mol_type = tRNA
caaaacatcagattgtgaatc

SEQ ID NO: 289 - RNA, 22 nt, Homo sapiens
  source 1..22: mol_type = tRNA
caaaacatcagattgtgaatct

SEQ ID NO: 290 - RNA, 24 nt, Homo sapiens
  source 1..24: mol_type = tRNA
caaaacatcagattgtgaatctga

SEQ ID NO: 291 - RNA, 25 nt, Homo sapiens
  source 1..25: mol_type = tRNA
caaaacatcagattgtgaatctgac

SEQ ID NO: 292 - RNA, 26 nt, Homo sapiens
  source 1..26: mol_type = tRNA
caaaacatcagattgtgaatctgaca

SEQ ID NO: 293 - RNA, 29 nt, Homo sapiens
  source 1..29: mol_type = tRNA
caaaacatcagattgtgaatctgacaaca

SEQ ID NO: 294 - RNA, 30 nt, Homo sapiens
  source 1..30: mol_type = tRNA
caaaacatcagattgtgaatctgacaacag

SEQ ID NO: 295 - RNA, 22 nt, Homo sapiens
  source 1..22: mol_type = tRNA
caacagaggcttacgacccctt

SEQ ID NO: 296 - RNA, 16 nt, Homo sapiens
  source 1..16: mol_type = tRNA
cagaggcttacgaccc

SEQ ID NO: 297 - RNA, 17 nt, Homo sapiens
  source 1..17: mol_type = tRNA
cagaggcttacgacccc

SEQ ID NO: 298 - RNA, 19 nt, Homo sapiens
  source 1..19: mol_type = tRNA
cagaggcttacgacccctt

SEQ ID NO: 299 - RNA, 16 nt, Homo sapiens
  source 1..16: mol_type = tRNA
cagattgtgaatctga

SEQ ID NO: 300 - RNA, 17 nt, Homo sapiens
  source 1..17: mol_type = tRNA
cagattgtgaatctgac

SEQ ID NO: 301 - RNA, 18 nt, Homo sapiens
  source 1..18: mol_type = tRNA
cagattgtgaatctgaca

SEQ ID NO: 302 - RNA, 19 nt, Homo sapiens
  source 1..19: mol_type = tRNA
cagattgtgaatctgacaa

SEQ ID NO: 303 - RNA, 20 nt, Homo sapiens
  source 1..20: mol_type = tRNA
cagattgtgaatctgacaac

SEQ ID NO: 304 - RNA, 21 nt, Homo sapiens
  source 1..21: mol_type = tRNA
cagattgtgaatctgacaaca

SEQ ID NO: 305 - RNA, 22 nt, Homo sapiens
  source 1..22: mol_type = tRNA
cagattgtgaatctgacaacag

SEQ ID NO: 306 - RNA, 23 nt, Homo sapiens
  source 1..23: mol_type = tRNA
cagattgtgaatctgacaacaga

SEQ ID NO: 307 - RNA, 24 nt, Homo sapiens
  source 1..24: mol_type = tRNA
cagattgtgaatctgacaacagag

SEQ ID NO: 308 - RNA, 26 nt, Homo sapiens
  source 1..26: mol_type = tRNA
cagattgtgaatctgacaacagaggc

SEQ ID NO: 309 - RNA, 27 nt, Homo sapiens
  source 1..27: mol_type = tRNA
cagattgtgaatctgacaacagaggct

SEQ ID NO: 310 - RNA, 28 nt, Homo sapiens
  source 1..28: mol_type = tRNA
cagattgtgaatctgacaacagaggctt

SEQ ID NO: 311 - RNA, 30 nt, Homo sapiens
  source 1..30: mol_type = tRNA
cagattgtgaatctgacaacagaggcttac

SEQ ID NO: 312 - RNA, 16 nt, Homo sapiens
  source 1..16: mol_type = tRNA
catcagattgtgaatc

SEQ ID NO: 313 - RNA, 17 nt, Homo sapiens
  source 1..17: mol_type = tRNA
catcagattgtgaatct

SEQ ID NO: 314 - RNA, 18 nt, Homo sapiens
  source 1..18: mol_type = tRNA
catcagattgtgaatctg

SEQ ID NO: 315 - RNA, 19 nt, Homo sapiens
  source 1..19: mol_type = tRNA
catcagattgtgaatctga

SEQ ID NO: 316 - RNA, 20 nt, Homo sapiens
  source 1..20: mol_type = tRNA
catcagattgtgaatctgac

SEQ ID NO: 317 - RNA, 21 nt, Homo sapiens
  source 1..21: mol_type = tRNA
catcagattgtgaatctgaca

SEQ ID NO: 318 - RNA, 22 nt, Homo sapiens
  source 1..22: mol_type = tRNA
catcagattgtgaatctgacaa

SEQ ID NO: 319 - RNA, 23 nt, Homo sapiens
  source 1..23: mol_type = tRNA
catcagattgtgaatctgacaac

SEQ ID NO: 320 - RNA, 24 nt, Homo sapiens
  source 1..24: mol_type = tRNA
catcagattgtgaatctgacaaca

SEQ ID NO: 321 - RNA, 25 nt, Homo sapiens
  source 1..25: mol_type = tRNA
catcagattgtgaatctgacaacag

SEQ ID NO: 322 - RNA, 26 nt, Homo sapiens
  source 1..26: mol_type = tRNA
catcagattgtgaatctgacaacaga

SEQ ID NO: 323 - RNA, 28 nt, Homo sapiens
  source 1..28: mol_type = tRNA
catcagattgtgaatctgacaacagagg

SEQ ID NO: 324 - RNA, 29 nt, Homo sapiens
  source 1..29: mol_type = tRNA
catcagattgtgaatctgacaacagaggc

SEQ ID NO: 325 - RNA, 30 nt, Homo sapiens
  source 1..30: mol_type = tRNA
catcagattgtgaatctgacaacagaggct

SEQ ID NO: 326 - RNA, 21 nt, Homo sapiens
  source 1..21: mol_type = tRNA
catgatcgtatagtggttagt

SEQ ID NO: 327 - RNA, 25 nt, Homo sapiens
  source 1..25: mol_type = tRNA
catgatcgtatagtggttagtactc

SEQ ID NO: 328 - RNA, 26 nt, Homo sapiens
  source 1..26: mol_type = tRNA
catgatcgtatagtggttagtactct

SEQ ID NO: 329 - RNA, 27 nt, Homo sapiens
  source 1..27: mol_type = tRNA
catgatcgtatagtggttagtactctg

SEQ ID NO: 330 - RNA, 28 nt, Homo sapiens
  source 1..28: mol_type = tRNA
catgatcgtatagtggttagtactctgc

SEQ ID NO: 331 - RNA, 17 nt, Homo sapiens
  source 1..17: mol_type = tRNA
ccaaaacatcagattgt

SEQ ID NO: 332 - RNA, 18 nt, Homo sapiens
  source 1..18: mol_type = tRNA
ccaaaacatcagattgtg

SEQ ID NO: 333 - RNA, 19 nt, Homo sapiens
  source 1..19: mol_type = tRNA
ccaaaacatcagattgtga

SEQ ID NO: 334 - RNA, 20 nt, Homo sapiens
  source 1..20: mol_type = tRNA
ccaaaacatcagattgtgaa

SEQ ID NO: 335 - RNA, 21 nt, Homo sapiens
  source 1..21: mol_type = tRNA
ccaaaacatcagattgtgaat

SEQ ID NO: 336 - RNA, 22 nt, Homo sapiens
  source 1..22: mol_type = tRNA
ccaaaacatcagattgtgaatc

SEQ ID NO: 337 - RNA, 23 nt, Homo sapiens
  source 1..23: mol_type = tRNA
ccaaaacatcagattgtgaatct

SEQ ID NO: 338 - RNA, 25 nt, Homo sapiens
  source 1..25: mol_type = tRNA
ccaaaacatcagattgtgaatctga

SEQ ID NO: 339 - RNA, 26 nt, Homo sapiens
  source 1..26: mol_type = tRNA
ccaaaacatcagattgtgaatctgac

SEQ ID NO: 340 - RNA, 27 nt, Homo sapiens
  source 1..27: mol_type = tRNA
ccaaaacatcagattgtgaatctgaca

SEQ ID NO: 341 - RNA, 29 nt, Homo sapiens
  source 1..29: mol_type = tRNA
ccaaaacatcagattgtgaatctgacaac

SEQ ID NO: 342 - RNA, 17 nt, Homo sapiens
  source 1..17: mol_type = tRNA
ccatgatcgtatagtgg

SEQ ID NO: 343 - RNA, 22 nt, Homo sapiens
  source 1..22: mol_type = tRNA
ccatgatcgtatagtggttagt

SEQ ID NO: 344 - RNA, 26 nt, Homo sapiens
  source 1..26: mol_type = tRNA
ccatgatcgtatagtggttagtactc

SEQ ID NO: 345 - RNA, 27 nt, Homo sapiens
  source 1..27: mol_type = tRNA
ccatgatcgtatagtggttagtactct

SEQ ID NO: 346 - RNA, 29 nt, Homo sapiens
  source 1..29: mol_type = tRNA
ccatgatcgtatagtggttagtactctgc

SEQ ID NO: 347 - RNA, 30 nt, Homo sapiens
  source 1..30: mol_type = tRNA
ccatgatcgtatagtggttagtactctgcg

SEQ ID NO: 348 - RNA, 18 nt, Homo sapiens
  source 1..18: mol_type = tRNA
ccgcagcaacctcggttc

SEQ ID NO: 349 - RNA, 19 nt, Homo sapiens
  source 1..19: mol_type = tRNA
ccgcagcaacctcggttcg

SEQ ID NO: 350 - RNA, 29 nt, Homo sapiens
  source 1..29: mol_type = tRNA
ccgcagcaacctcggttcgaatccgagtc

SEQ ID NO: 351 - RNA, 16 nt, Homo sapiens
  source 1..16: mol_type = tRNA
ccgtgatcgtatagtg

SEQ ID NO: 352 - RNA, 17 nt, Homo sapiens
  source 1..17: mol_type = tRNA
ccgtgatcgtatagtgg

SEQ ID NO: 353 - RNA, 18 nt, Homo sapiens
  source 1..18: mol_type = tRNA
ccgtgatcgtatagtggt

SEQ ID NO: 354 - RNA, 19 nt, Homo sapiens
  source 1..19: mol_type = tRNA
ccgtgatcgtatagtggtt

SEQ ID NO: 355 - RNA, 20 nt, Homo sapiens
  source 1..20: mol_type = tRNA
ccgtgatcgtatagtggtta

SEQ ID NO: 356 - RNA, 21 nt, Homo sapiens
  source 1..21: mol_type = tRNA
ccgtgatcgtatagtggttag

SEQ ID NO: 357 - RNA, 22 nt, Homo sapiens
  source 1..22: mol_type = tRNA
ccgtgatcgtatagtggttagt

SEQ ID NO: 358 - RNA, 23 nt, Homo sapiens
  source 1..23: mol_type = tRNA
ccgtgatcgtatagtggttagta

SEQ ID NO: 359 - RNA, 24 nt, Homo sapiens
  source 1..24: mol_type = tRNA
ccgtgatcgtatagtggttagtac

SEQ ID NO: 360 - RNA, 25 nt, Homo sapiens
  source 1..25: mol_type = tRNA
ccgtgatcgtatagtggttagtact

SEQ ID NO: 361 - RNA, 26 nt, Homo sapiens
  source 1..26: mol_type = tRNA
ccgtgatcgtatagtggttagtactc

SEQ ID NO: 362 - RNA, 27 nt, Homo sapiens
  source 1..27: mol_type = tRNA
ccgtgatcgtatagtggttagtactct

SEQ ID NO: 363 - RNA, 28 nt, Homo sapiens
  source 1..28: mol_type = tRNA
ccgtgatcgtatagtggttagtactctg

SEQ ID NO: 364 - RNA, 29 nt, Homo sapiens
  source 1..29: mol_type = tRNA
ccgtgatcgtatagtggttagtactctgc

SEQ ID NO: 365 - RNA, 30 nt, Homo sapiens
  source 1..30: mol_type = tRNA
ccgtgatcgtatagtggttagtactctgcg

SEQ ID NO: 366 - RNA, 16 nt, Homo sapiens
  source 1..16: mol_type = tRNA
cgaccccttatttacc

SEQ ID NO: 367 - RNA, 17 nt, Homo sapiens
  source 1..17: mol_type = tRNA
cgcagcaacctcggttc

SEQ ID NO: 368 - RNA, 18 nt, Homo sapiens
  source 1..18: mol_type = tRNA
cgcagcaacctcggttcg

SEQ ID NO: 369 - RNA, 30 nt, Homo sapiens
  source 1..30: mol_type = tRNA
cgcagcaacctcggttcgaatccgagtcac

SEQ ID NO: 370 - RNA, 18 nt, Homo sapiens
  source 1..18: mol_type = tRNA
cgtatagtggttagtact

SEQ ID NO: 371 - RNA, 19 nt, Homo sapiens
  source 1..19: mol_type = tRNA
cgtatagtggttagtactc

SEQ ID NO: 372 - RNA, 20 nt, Homo sapiens
  source 1..20: mol_type = tRNA
cgtatagtggttagtactct

SEQ ID NO: 373 - RNA, 21 nt, Homo sapiens
  source 1..21: mol_type = tRNA
cgtatagtggttagtactctg

SEQ ID NO: 374 - RNA, 22 nt, Homo sapiens
  source 1..22: mol_type = tRNA
cgtatagtggttagtactctgc

SEQ ID NO: 375 - RNA, 23 nt, Homo sapiens
  source 1..23: mol_type = tRNA
cgtatagtggttagtactctgcg

SEQ ID NO: 376 - RNA, 24 nt, Homo sapiens
  source 1..24: mol_type = tRNA
cgtatagtggttagtactctgcgc

SEQ ID NO: 377 - RNA, 25 nt, Homo sapiens
  source 1..25: mol_type = tRNA
cgtatagtggttagtactctgcgct

SEQ ID NO: 378 - RNA, 26 nt, Homo sapiens
  source 1..26: mol_type = tRNA
cgtatagtggttagtactctgcgctg

SEQ ID NO: 379 - RNA, 24 nt, Homo sapiens
  source 1..24: mol_type = tRNA
cgtatagtggttagtactctgcgt

SEQ ID NO: 380 - RNA, 25 nt, Homo sapiens
  source 1..25: mol_type = tRNA
cgtatagtggttagtactctgcgtt

SEQ ID NO: 381 - RNA, 26 nt, Homo sapiens
  source 1..26: mol_type = tRNA
cgtatagtggttagtactctgcgttg

SEQ ID NO: 382 - RNA, 27 nt, Homo sapiens
  source 1..27: mol_type = tRNA
cgtatagtggttagtactctgcgttgt

SEQ ID NO: 383 - RNA, 28 nt, Homo sapiens
  source 1..28: mol_type = tRNA
cgtatagtggttagtactctgcgttgtg

SEQ ID NO: 384 - RNA, 30 nt, Homo sapiens
  source 1..30: mol_type = tRNA
cgtatagtggttagtactctgcgttgtggc

SEQ ID NO: 385 - RNA, 16 nt, Homo sapiens
  source 1..16: mol_type = tRNA
cgtgatcgtatagtgg

SEQ ID NO: 386 - RNA, 17 nt, Homo sapiens
  source 1..17: mol_type = tRNA
cgtgatcgtatagtggt

SEQ ID NO: 387 - RNA, 18 nt, Homo sapiens
  source 1..18: mol_type = tRNA
cgtgatcgtatagtggtt

SEQ ID NO: 388 - RNA, 19 nt, Homo sapiens
  source 1..19: mol_type = tRNA
cgtgatcgtatagtggtta

SEQ ID NO: 389 - RNA, 20 nt, Homo sapiens
  source 1..20: mol_type = tRNA
cgtgatcgtatagtggttag

SEQ ID NO: 390 - RNA, 21 nt, Homo sapiens
  source 1..21: mol_type = tRNA
cgtgatcgtatagtggttagt

SEQ ID NO: 391 - RNA, 22 nt, Homo sapiens
  source 1..22: mol_type = tRNA
cgtgatcgtatagtggttagta

SEQ ID NO: 392 - RNA, 23 nt, Homo sapiens
  source 1..23: mol_type = tRNA
cgtgatcgtatagtggttagtac

SEQ ID NO: 393 - RNA, 24 nt, Homo sapiens
  source 1..24: mol_type = tRNA
cgtgatcgtatagtggttagtact

SEQ ID NO: 394 - RNA, 25 nt, Homo sapiens
  source 1..25: mol_type = tRNA
cgtgatcgtatagtggttagtactc

SEQ ID NO: 395 - RNA, 26 nt, Homo sapiens
  source 1..26: mol_type = tRNA
cgtgatcgtatagtggttagtactct

SEQ ID NO: 396 - RNA, 27 nt, Homo sapiens
  source 1..27: mol_type = tRNA
cgtgatcgtatagtggttagtactctg

SEQ ID NO: 397 - RNA, 28 nt, Homo sapiens
  source 1..28: mol_type = tRNA
cgtgatcgtatagtggttagtactctgc

SEQ ID NO: 398 - RNA, 29 nt, Homo sapiens
  source 1..29: mol_type = tRNA
cgtgatcgtatagtggttagtactctgcg

SEQ ID NO: 399 - RNA, 30 nt, Homo sapiens
  source 1..30: mol_type = tRNA
cgtgatcgtatagtggttagtactctgcgt

SEQ ID NO: 400 - RNA, 19 nt, Homo sapiens
  source 1..19: mol_type = tRNA
cgttgtggccgcagcaacc

SEQ ID NO: 401 - RNA, 24 nt, Homo sapiens
  source 1..24: mol_type = tRNA
cgttgtggccgcagcaacctcggt

SEQ ID NO: 402 - RNA, 18 nt, Homo sapiens
  source 1..18: mol_type = tRNA
ctcggttcgaatccgagt

SEQ ID NO: 403 - RNA, 22 nt, Homo sapiens
  source 1..22: mol_type = tRNA
ctcggttcgaatccgagtcacg

SEQ ID NO: 404 - RNA, 23 nt, Homo sapiens
  source 1..23: mol_type = tRNA
ctcggttcgaatccgagtcacgg

SEQ ID NO: 405 - RNA, 16 nt, Homo sapiens
  source 1..16: mol_type = tRNA
ctctgcgttgtggccg

SEQ ID NO: 406 - RNA, 17 nt, Homo sapiens
  source 1..17: mol_type = tRNA
ctctgcgttgtggccgc

SEQ ID NO: 407 - RNA, 18 nt, Homo sapiens
  source 1..18: mol_type = tRNA
ctctgcgttgtggccgca

SEQ ID NO: 408 - RNA, 19 nt, Homo sapiens
  source 1..19: mol_type = tRNA
ctctgcgttgtggccgcag

SEQ ID NO: 409 - RNA, 20 nt, Homo sapiens
  source 1..20: mol_type = tRNA
ctctgcgttgtggccgcagc

SEQ ID NO: 410 - RNA, 21 nt, Homo sapiens
  source 1..21: mol_type = tRNA
ctctgcgttgtggccgcagca

SEQ ID NO: 411 - RNA, 23 nt, Homo sapiens
  source 1..23: mol_type = tRNA
ctctgcgttgtggccgcagcaac

SEQ ID NO: 412 - RNA, 24 nt, Homo sapiens
  source 1..24: mol_type = tRNA
ctctgcgttgtggccgcagcaacc

SEQ ID NO: 413 - RNA, 28 nt, Homo sapiens
  source 1..28: mol_type = tRNA
ctctgcgttgtggccgcagcaacctcgg

SEQ ID NO: 414 - RNA, 29 nt, Homo sapiens
  source 1..29: mol_type = tRNA
ctctgcgttgtggccgcagcaacctcggt

SEQ ID NO: 415 - RNA, 18 nt, Homo sapiens
  source 1..18: mol_type = tRNA
ctgacaacagaggcttac

SEQ ID NO: 416 - RNA, 23 nt, Homo sapiens
  source 1..23: mol_type = tRNA
ctgacaacagaggcttacgaccc

SEQ ID NO: 417 - RNA, 27 nt, Homo sapiens
  source 1..27: mol_type = tRNA
ctgacaacagaggcttacgacccctta

SEQ ID NO: 418 - RNA, 30 nt, Homo sapiens
  source 1..30: mol_type = tRNA
ctgacaacagaggcttacgaccccttattt

SEQ ID NO: 419 - RNA, 24 nt, Homo sapiens
  source 1..24: mol_type = tRNA
ctgcgttgtggccgcagcaacctc

SEQ ID NO: 420 - RNA, 25 nt, Homo sapiens
  source 1..25: mol_type = tRNA
ctgcgttgtggccgcagcaacctcg

SEQ ID NO: 421 - RNA, 26 nt, Homo sapiens
  source 1..26: mol_type = tRNA
ctgcgttgtggccgcagcaacctcgg

SEQ ID NO: 422 - RNA, 16 nt, Homo sapiens
  source 1..16: mol_type = tRNA
cttacgaccccttatt

SEQ ID NO: 423 - RNA, 17 nt, Homo sapiens
  source 1..17: mol_type = tRNA
cttacgaccccttattt

SEQ ID NO: 424 - RNA, 18 nt, Homo sapiens
  source 1..18: mol_type = tRNA
cttacgaccccttattta

SEQ ID NO: 425 - RNA, 19 nt, Homo sapiens
  source 1..19: mol_type = tRNA
cttacgaccccttatttac

SEQ ID NO: 426 - RNA, 20 nt, Homo sapiens
  source 1..20: mol_type = tRNA
cttacgaccccttatttacc

SEQ ID NO: 427 - RNA, 18 nt, Homo sapiens
  source 1..18: mol_type = tRNA
gaatctgacaacagaggc

SEQ ID NO: 428 - RNA, 19 nt, Homo sapiens
  source 1..19: mol_type = tRNA
gaatctgacaacagaggct

SEQ ID NO: 429 - RNA, 20 nt, Homo sapiens
  source 1..20: mol_type = tRNA
gaatctgacaacagaggctt

SEQ ID NO: 430 - RNA, 21 nt, Homo sapiens
  source 1..21: mol_type = tRNA
gaatctgacaacagaggctta

SEQ ID NO: 431 - RNA, 23 nt, Homo sapiens
  source 1..23: mol_type = tRNA
gaatctgacaacagaggcttacg

SEQ ID NO: 432 - RNA, 30 nt, Homo sapiens
  source 1..30: mol_type = tRNA
gaatctgacaacagaggcttacgacccctt

SEQ ID NO: 433 - RNA, 16 nt, Homo sapiens
  source 1..16: mol_type = tRNA
gacaacagaggcttac

SEQ ID NO: 434 - RNA, 22 nt, Homo sapiens
  source 1..22: mol_type = tRNA
gacaacagaggcttacgacccc

SEQ ID NO: 435 - RNA, 24 nt, Homo sapiens
  source 1..24: mol_type = tRNA
gacaacagaggcttacgacccctt

SEQ ID NO: 436 - RNA, 25 nt, Homo sapiens
  source 1..25: mol_type = tRNA
gacaacagaggcttacgacccctta

SEQ ID NO: 437 - RNA, 30 nt, Homo sapiens
  source 1..30: mol_type = tRNA
gacaacagaggcttacgaccccttatttac

SEQ ID NO: 438 - RNA, 16 nt, Homo sapiens
  source 1..16: mol_type = tRNA
gaggcttacgacccct

SEQ ID NO: 439 - RNA, 17 nt, Homo sapiens
  source 1..17: mol_type = tRNA
gaggcttacgacccctt

SEQ ID NO: 440 - RNA, 18 nt, Homo sapiens
  source 1..18: mol_type = tRNA
gaggcttacgacccctta

SEQ ID NO: 441 - RNA, 19 nt, Homo sapiens
  source 1..19: mol_type = tRNA
gaggcttacgaccccttat

SEQ ID NO: 442 - RNA, 20 nt, Homo sapiens
  source 1..20: mol_type = tRNA
gaggcttacgaccccttatt

SEQ ID NO: 443 - RNA, 21 nt, Homo sapiens
  source 1..21: mol_type = tRNA
gaggcttacgaccccttattt

SEQ ID NO: 444 - RNA, 22 nt, Homo sapiens
  source 1..22: mol_type = tRNA
gaggcttacgaccccttattta

SEQ ID NO: 445 - RNA, 23 nt, Homo sapiens
  source 1..23: mol_type = tRNA
gaggcttacgaccccttatttac

SEQ ID NO: 446 - RNA, 24 nt, Homo sapiens
  source 1..24: mol_type = tRNA
gaggcttacgaccccttatttacc

SEQ ID NO: 447 - RNA, 17 nt, Homo sapiens
  source 1..17: mol_type = tRNA
gatcgtatagtggttag

SEQ ID NO: 448 - RNA, 18 nt, Homo sapiens
  source 1..18: mol_type = tRNA
gatcgtatagtggttagt

SEQ ID NO: 449 - RNA, 19 nt, Homo sapiens
  source 1..19: mol_type = tRNA
gatcgtatagtggttagta

SEQ ID NO: 450 - RNA, 20 nt, Homo sapiens
  source 1..20: mol_type = tRNA
gatcgtatagtggttagtac

SEQ ID NO: 451 - RNA, 21 nt, Homo sapiens
  source 1..21: mol_type = tRNA
gatcgtatagtggttagtact

SEQ ID NO: 452 - RNA, 22 nt, Homo sapiens
  source 1..22: mol_type = tRNA
gatcgtatagtggttagtactc

SEQ ID NO: 453 - RNA, 23 nt, Homo sapiens
  source 1..23: mol_type = tRNA
gatcgtatagtggttagtactct

SEQ ID NO: 454 - RNA, 24 nt, Homo sapiens
  source 1..24: mol_type = tRNA
gatcgtatagtggttagtactctg

SEQ ID NO: 455 - RNA, 25 nt, Homo sapiens
  source 1..25: mol_type = tRNA
gatcgtatagtggttagtactctgc

SEQ ID NO: 456 - RNA, 26 nt, Homo sapiens
  source 1..26: mol_type = tRNA
gatcgtatagtggttagtactctgcg

SEQ ID NO: 457 - RNA, 27 nt, Homo sapiens
  source 1..27: mol_type = tRNA
gatcgtatagtggttagtactctgcgt

SEQ ID NO: 458 - RNA, 28 nt, Homo sapiens
  source 1..28: mol_type = tRNA
gatcgtatagtggttagtactctgcgtt

SEQ ID NO: 459 - RNA, 29 nt, Homo sapiens
  source 1..29: mol_type = tRNA
gatcgtatagtggttagtactctgcgttg

SEQ ID NO: 460 - RNA, 30 nt, Homo sapiens
  source 1..30: mol_type = tRNA
gatcgtatagtggttagtactctgcgttgt

SEQ ID NO: 461 - RNA, 16 nt, Homo sapiens
  source 1..16: mol_type = tRNA
gattgtgaatctgaca

SEQ ID NO: 462 - RNA, 17 nt, Homo sapiens
  source 1..17: mol_type = tRNA
gattgtgaatctgacaa

SEQ ID NO: 463 - RNA, 18 nt, Homo sapiens
  source 1..18: mol_type = tRNA
gattgtgaatctgacaac

SEQ ID NO: 464 - RNA, 19 nt, Homo sapiens
  source 1..19: mol_type = tRNA
gattgtgaatctgacaaca

SEQ ID NO: 465 - RNA, 20 nt, Homo sapiens
  source 1..20: mol_type = tRNA
gattgtgaatctgacaacag

SEQ ID NO: 466 - RNA, 21 nt, Homo sapiens
  source 1..21: mol_type = tRNA
gattgtgaatctgacaacaga

SEQ ID NO: 467 - RNA, 22 nt, Homo sapiens
  source 1..22: mol_type = tRNA
gattgtgaatctgacaacagag

SEQ ID NO: 468 - RNA, 23 nt, Homo sapiens
  source 1..23: mol_type = tRNA
gattgtgaatctgacaacagagg

SEQ ID NO: 469 - RNA, 24 nt, Homo sapiens
  source 1..24: mol_type = tRNA
gattgtgaatctgacaacagaggc

SEQ ID NO: 470 - RNA, 25 nt, Homo sapiens
  source 1..25: mol_type = tRNA
gattgtgaatctgacaacagaggct

SEQ ID NO: 471 - RNA, 26 nt, Homo sapiens
  source 1..26: mol_type = tRNA
gattgtgaatctgacaacagaggctt

SEQ ID NO: 472 - RNA, 27 nt, Homo sapiens
  source 1..27: mol_type = tRNA
gattgtgaatctgacaacagaggctta

SEQ ID NO: 473 - RNA, 28 nt, Homo sapiens
  source 1..28: mol_type = tRNA
gattgtgaatctgacaacagaggcttac

SEQ ID NO: 474 - RNA, 29 nt, Homo sapiens
  source 1..29: mol_type = tRNA
gattgtgaatctgacaacagaggcttacg

SEQ ID NO: 475 - RNA, 27 nt, Homo sapiens
  source 1..27: mol_type = tRNA
gcaacctcggttcgaatccgagtcacg

SEQ ID NO: 476 - RNA, 28 nt, Homo sapiens
  source 1..28: mol_type = tRNA
gcaacctcggttcgaatccgagtcacgg

SEQ ID NO: 477 - RNA, 29 nt, Homo sapiens
  source 1..29: mol_type = tRNA
gcaacctcggttcgaatccgagtcacggc

SEQ ID NO: 478 - RNA, 16 nt, Homo sapiens
  source 1..16: mol_type = tRNA
gcagcaacctcggttc

SEQ ID NO: 479 - RNA, 17 nt, Homo sapiens
  source 1..17: mol_type = tRNA
gcagcaacctcggttcg

SEQ ID NO: 480 - RNA, 19 nt, Homo sapiens
  source 1..19: mol_type = tRNA
gcagcaacctcggttcgaa

SEQ ID NO: 481 - RNA, 21 nt, Homo sapiens
  source 1..21: mol_type = tRNA
gcagcaacctcggttcgaatc

SEQ ID NO: 482 - RNA, 26 nt, Homo sapiens
  source 1..26: mol_type = tRNA
gcagcaacctcggttcgaatccgagt

SEQ ID NO: 483 - RNA, 27 nt, Homo sapiens
  source 1..27: mol_type = tRNA
gcagcaacctcggttcgaatccgagtc

SEQ ID NO: 484 - RNA, 28 nt, Homo sapiens
  source 1..28: mol_type = tRNA
gcagcaacctcggttcgaatccgagtca

SEQ ID NO: 485 - RNA, 29 nt, Homo sapiens
  source 1..29: mol_type = tRNA
gcagcaacctcggttcgaatccgagtcac

SEQ ID NO: 486 - RNA, 30 nt, Homo sapiens
  source 1..30: mol_type = tRNA
gcagcaacctcggttcgaatccgagtcacg

SEQ ID NO: 487 - RNA, 30 nt, Homo sapiens
  source 1..30: mol_type = tRNA
gccgcagcaacctcggttcgaatccgagtc

SEQ ID NO: 488 - RNA, 20 nt, Homo sapiens
  source 1..20: mol_type = tRNA
gcgttgtggccgcagcaacc

SEQ ID NO: 489 - RNA, 23 nt, Homo sapiens
  source 1..23: mol_type = tRNA
gcgttgtggccgcagcaacctcg

SEQ ID NO: 490 - RNA, 24 nt, Homo sapiens
  source 1..24: mol_type = tRNA
gcgttgtggccgcagcaacctcgg

SEQ ID NO: 491 - RNA, 17 nt, Homo sapiens
  source 1..17: mol_type = tRNA
gcttacgaccccttatt

SEQ ID NO: 492 - RNA, 18 nt, Homo sapiens
  source 1..18: mol_type = tRNA
gcttacgaccccttattt

SEQ ID NO: 493 - RNA, 20 nt, Homo sapiens
  source 1..20: mol_type = tRNA
gcttacgaccccttatttac

SEQ ID NO: 494 - RNA, 21 nt, Homo sapiens
  source 1..21: mol_type = tRNA
gcttacgaccccttatttacc

SEQ ID NO: 495 - RNA, 17 nt, Homo sapiens
  source 1..17: mol_type = tRNA
ggccgcagcaacctcgg

SEQ ID NO: 496 - RNA, 18 nt, Homo sapiens
  source 1..18: mol_type = tRNA
ggccgcagcaacctcggt

SEQ ID NO: 497 - RNA, 20 nt, Homo sapiens
  source 1..20: mol_type = tRNA
ggccgcagcaacctcggttc

SEQ ID NO: 498 - RNA, 21 nt, Homo sapiens
  source 1..21: mol_type = tRNA
ggccgcagcaacctcggttcg

SEQ ID NO: 499 - RNA, 16 nt, Homo sapiens
  source 1..16: mol_type = tRNA
ggcttacgacccctta

SEQ ID NO: 500 - RNA, 19 nt, Homo sapiens
  source 1..19: mol_type = tRNA
ggcttacgaccccttattt

SEQ ID NO: 501 - RNA, 22 nt, Homo sapiens
  source 1..22: mol_type = tRNA
ggcttacgaccccttatttacc

SEQ ID NO: 502 - RNA, 16 nt, Homo sapiens
  source 1..16: mol_type = tRNA
ggttagtactctgcgc

SEQ ID NO: 503 - RNA, 17 nt, Homo sapiens
  source 1..17: mol_type = tRNA
ggttagtactctgcgct

SEQ ID NO: 504 - RNA, 18 nt, Homo sapiens
  source 1..18: mol_type = tRNA
ggttagtactctgcgctg

SEQ ID NO: 505 - RNA, 16 nt, Homo sapiens
  source 1..16: mol_type = tRNA
ggttagtactctgcgt

SEQ ID NO: 506 - RNA, 17 nt, Homo sapiens
  source 1..17: mol_type = tRNA
ggttagtactctgcgtt

SEQ ID NO: 507 - RNA, 18 nt, Homo sapiens
  source 1..18: mol_type = tRNA
ggttagtactctgcgttg

SEQ ID NO: 508 - RNA, 19 nt, Homo sapiens
  source 1..19: mol_type = tRNA
ggttagtactctgcgttgt

SEQ ID NO: 509 - RNA, 20 nt, Homo sapiens
  source 1..20: mol_type = tRNA
ggttagtactctgcgttgtg

SEQ ID NO: 510 - RNA, 21 nt, Homo sapiens
  source 1..21: mol_type = tRNA
ggttagtactctgcgttgtgg

SEQ ID NO: 511 - RNA, 22 nt, Homo sapiens
  source 1..22: mol_type = tRNA
ggttagtactctgcgttgtggc

SEQ ID NO: 512 - RNA, 23 nt, Homo sapiens
  source 1..23: mol_type = tRNA
ggttagtactctgcgttgtggcc

SEQ ID NO: 513 - RNA, 24 nt, Homo sapiens
  source 1..24: mol_type = tRNA
ggttagtactctgcgttgtggccg

SEQ ID NO: 514 - RNA, 25 nt, Homo sapiens
  source 1..25: mol_type = tRNA
ggttagtactctgcgttgtggccgc

SEQ ID NO: 515 - RNA, 28 nt, Homo sapiens
  source 1..28: mol_type = tRNA
ggttagtactctgcgttgtggccgcagc

SEQ ID NO: 516 - RNA, 16 nt, Homo sapiens
  source 1..16: mol_type = tRNA
ggttcgaatccgagtc

SEQ ID NO: 517 - RNA, 18 nt, Homo sapiens
  source 1..18: mol_type = tRNA
ggttcgaatccgagtcac

SEQ ID NO: 518 - RNA, 19 nt, Homo sapiens
  source 1..19: mol_type = tRNA
ggttcgaatccgagtcacg

SEQ ID NO: 519 - RNA, 20 nt, Homo sapiens
  source 1..20: mol_type = tRNA
ggttcgaatccgagtcacgg

SEQ ID NO: 520 - RNA, 22 nt, Homo sapiens
  source 1..22: mol_type = tRNA
ggttcgaatccgagtcacggca

SEQ ID NO: 521 - RNA, 17 nt, Homo sapiens
  source 1..17: mol_type = tRNA
gtactctgcgttgtggc

SEQ ID NO: 522 - RNA, 18 nt, Homo sapiens
  source 1..18: mol_type = tRNA
gtactctgcgttgtggcc

SEQ ID NO: 523 - RNA, 19 nt, Homo sapiens
  source 1..19: mol_type = tRNA
gtactctgcgttgtggccg

SEQ ID NO: 524 - RNA, 20 nt, Homo sapiens
  source 1..20: mol_type = tRNA
gtactctgcgttgtggccgc

SEQ ID NO: 525 - RNA, 23 nt, Homo sapiens
  source 1..23: mol_type = tRNA
gtactctgcgttgtggccgcagc

SEQ ID NO: 526 - RNA, 20 nt, Homo sapiens
  source 1..20: mol_type = tRNA
gtatagtggttagcactctg

SEQ ID NO: 527 - RNA, 16 nt, Homo sapiens
  source 1..16: mol_type = tRNA
gtatagtggttagtac

SEQ ID NO: 528 - RNA, 17 nt, Homo sapiens
  source 1..17: mol_type = tRNA
gtatagtggttagtact

SEQ ID NO: 529 - RNA, 18 nt, Homo sapiens
  source 1..18: mol_type = tRNA
gtatagtggttagtactc

SEQ ID NO: 530 - RNA, 19 nt, Homo sapiens
  source 1..19: mol_type = tRNA
gtatagtggttagtactct

SEQ ID NO: 531 - RNA, 20 nt, Homo sapiens
  source 1..20: mol_type = tRNA
gtatagtggttagtactctg

SEQ ID NO: 532 - RNA, 21 nt, Homo sapiens
  source 1..21: mol_type = tRNA
gtatagtggttagtactctgc

SEQ ID NO: 533 - RNA, 22 nt, Homo sapiens
  source 1..22: mol_type = tRNA
gtatagtggttagtactctgcg

SEQ ID NO: 534 - RNA, 23 nt, Homo sapiens
  source 1..23: mol_type = tRNA
gtatagtggttagtactctgcgt

SEQ ID NO: 535 - RNA, 24 nt, Homo sapiens
  source 1..24: mol_type = tRNA
gtatagtggttagtactctgcgtt

SEQ ID NO: 536 - RNA, 25 nt, Homo sapiens
  source 1..25: mol_type = tRNA
gtatagtggttagtactctgcgttg

SEQ ID NO: 537 - RNA, 26 nt, Homo sapiens
  source 1..26: mol_type = tRNA
gtatagtggttagtactctgcgttgt

SEQ ID NO: 538 - RNA, 27 nt, Homo sapiens
  source 1..27: mol_type = tRNA
gtatagtggttagtactctgcgttgtg

SEQ ID NO: 539 - RNA, 29 nt, Homo sapiens
  source 1..29: mol_type = tRNA
gtatagtggttagtactctgcgttgtggc

SEQ ID NO: 540 - RNA, 30 nt, Homo sapiens
  source 1..30: mol_type = tRNA
gtatagtggttagtactctgcgttgtggcc

SEQ ID NO: 541 - RNA, 16 nt, Homo sapiens
  source 1..16: mol_type = tRNA
gtgaatctgacaacag

SEQ ID NO: 542 - RNA, 18 nt, Homo sapiens
  source 1..18: mol_type = tRNA
gtgaatctgacaacagag

SEQ ID NO: 543 - RNA, 19 nt, Homo sapiens
  source 1..19: mol_type = tRNA
gtgaatctgacaacagagg

SEQ ID NO: 544 - RNA, 20 nt, Homo sapiens
  source 1..20: mol_type = tRNA
gtgaatctgacaacagaggc

SEQ ID NO: 545 - RNA, 21 nt, Homo sapiens
  source 1..21: mol_type = tRNA
gtgaatctgacaacagaggct

SEQ ID NO: 546 - RNA, 22 nt, Homo sapiens
  source 1..22: mol_type = tRNA
gtgaatctgacaacagaggctt

SEQ ID NO: 547 - RNA, 23 nt, Homo sapiens
  source 1..23: mol_type = tRNA
gtgaatctgacaacagaggctta

SEQ ID NO: 548 - RNA, 24 nt, Homo sapiens
  source 1..24: mol_type = tRNA
gtgaatctgacaacagaggcttac

SEQ ID NO: 549 - RNA, 25 nt, Homo sapiens
  source 1..25: mol_type = tRNA
gtgaatctgacaacagaggcttacg

SEQ ID NO: 550 - RNA, 26 nt, Homo sapiens
  source 1..26: mol_type = tRNA
gtgaatctgacaacagaggcttacga

SEQ ID NO: 551 - RNA, 27 nt, Homo sapiens
  source 1..27: mol_type = tRNA
gtgaatctgacaacagaggcttacgac

SEQ ID NO: 552 - RNA, 28 nt, Homo sapiens
  source 1..28: mol_type = tRNA
gtgaatctgacaacagaggcttacgacc

SEQ ID NO: 553 - RNA, 29 nt, Homo sapiens
  source 1..29: mol_type = tRNA
gtgaatctgacaacagaggcttacgaccc

SEQ ID NO: 554 - RNA, 30 nt, Homo sapiens
  source 1..30: mol_type = tRNA
gtgaatctgacaacagaggcttacgacccc

SEQ ID NO: 555 - RNA, 16 nt, Homo sapiens
  source 1..16: mol_type = tRNA
gtgatcgtatagtggt

SEQ ID NO: 556 - RNA, 17 nt, Homo sapiens
  source 1..17: mol_type = tRNA
gtgatcgtatagtggtt

SEQ ID NO: 557 - RNA, 18 nt, Homo sapiens
  source 1..18: mol_type = tRNA
gtgatcgtatagtggtta

SEQ ID NO: 558 - RNA, 19 nt, Homo sapiens
  source 1..19: mol_type = tRNA
gtgatcgtatagtggttag

SEQ ID NO: 559 - RNA, 20 nt, Homo sapiens
  source 1..20: mol_type = tRNA
gtgatcgtatagtggttagt

SEQ ID NO: 560 - RNA, 21 nt, Homo sapiens
  source 1..21: mol_type = tRNA
gtgatcgtatagtggttagta

SEQ ID NO: 561 - RNA, 22 nt, Homo sapiens
  source 1..22: mol_type = tRNA
gtgatcgtatagtggttagtac

SEQ ID NO: 562 - RNA, 23 nt, Homo sapiens
  source 1..23: mol_type = tRNA
gtgatcgtatagtggttagtact

SEQ ID NO: 563 - RNA, 24 nt, Homo sapiens
  source 1..24: mol_type = tRNA
gtgatcgtatagtggttagtactc

SEQ ID NO: 564 - RNA, 25 nt, Homo sapiens
  source 1..25: mol_type = tRNA
gtgatcgtatagtggttagtactct

SEQ ID NO: 565 - RNA, 26 nt, Homo sapiens
  source 1..26: mol_type = tRNA
gtgatcgtatagtggttagtactctg

SEQ ID NO: 566 - RNA, 27 nt, Homo sapiens
  source 1..27: mol_type = tRNA
gtgatcgtatagtggttagtactctgc

SEQ ID NO: 567 - RNA, 28 nt, Homo sapiens
  source 1..28: mol_type = tRNA
gtgatcgtatagtggttagtactctgcg

SEQ ID NO: 568 - RNA, 29 nt, Homo sapiens
  source 1..29: mol_type = tRNA
gtgatcgtatagtggttagtactctgcgt

SEQ ID NO: 569 - RNA, 30 nt, Homo sapiens
  source 1..30: mol_type = tRNA
gtgatcgtatagtggttagtactctgcgtt

SEQ ID NO: 570 - RNA, 17 nt, Homo sapiens
  source 1..17: mol_type = tRNA
gtggccgcagcaacctc

SEQ ID NO: 571 - RNA, 18 nt, Homo sapiens
  source 1..18: mol_type = tRNA
gtggccgcagcaacctcg

SEQ ID NO: 572 - RNA, 19 nt, Homo sapiens
  source 1..19: mol_type = tRNA
gtggccgcagcaacctcgg

SEQ ID NO: 573 - RNA, 20 nt, Homo sapiens
  source 1..20: mol_type = tRNA
gtggccgcagcaacctcggt

SEQ ID NO: 574 - RNA, 22 nt, Homo sapiens
  source 1..22: mol_type = tRNA
gtggccgcagcaacctcggttc

SEQ ID NO: 575 - RNA, 23 nt, Homo sapiens
  source 1..23: mol_type = tRNA
gtggccgcagcaacctcggttcg

SEQ ID NO: 576 - RNA, 25 nt, Homo sapiens
  source 1..25: mol_type = tRNA
gtggccgcagcaacctcggttcgaa

SEQ ID NO: 577 - RNA, 16 nt, Homo sapiens
  source 1..16: mol_type = tRNA
gtggttagtactctgc

SEQ ID NO: 578 - RNA, 17 nt, Homo sapiens
  source 1..17: mol_type = tRNA
gtggttagtactctgcg

SEQ ID NO: 579 - RNA, 18 nt, Homo sapiens
  source 1..18: mol_type = tRNA
gtggttagtactctgcgc

SEQ ID NO: 580 - RNA, 19 nt, Homo sapiens
  source 1..19: mol_type = tRNA
gtggttagtactctgcgct

SEQ ID NO: 581 - RNA, 20 nt, Homo sapiens
  source 1..20: mol_type = tRNA
gtggttagtactctgcgctg

SEQ ID NO: 582 - RNA, 21 nt, Homo sapiens
  source 1..21: mol_type = tRNA
gtggttagtactctgcgctgt

SEQ ID NO: 583 - RNA, 22 nt, Homo sapiens
  source 1..22: mol_type = tRNA
gtggttagtactctgcgctgtg

SEQ ID NO: 584 - RNA, 18 nt, Homo sapiens
  source 1..18: mol_type = tRNA
gtggttagtactctgcgt

SEQ ID NO: 585 - RNA, 19 nt, Homo sapiens
  source 1..19: mol_type = tRNA
gtggttagtactctgcgtt

SEQ ID NO: 586 - RNA, 20 nt, Homo sapiens
  source 1..20: mol_type = tRNA
gtggttagtactctgcgttg

SEQ ID NO: 587 - RNA, 21 nt, Homo sapiens
  source 1..21: mol_type = tRNA
gtggttagtactctgcgttgt

SEQ ID NO: 588 - RNA, 22 nt, Homo sapiens
  source 1..22: mol_type = tRNA
gtggttagtactctgcgttgtg

SEQ ID NO: 589 - RNA, 23 nt, Homo sapiens
  source 1..23: mol_type = tRNA
gtggttagtactctgcgttgtgg

SEQ ID NO: 590 - RNA, 24 nt, Homo sapiens
  source 1..24: mol_type = tRNA
gtggttagtactctgcgttgtggc

SEQ ID NO: 591 - RNA, 25 nt, Homo sapiens
  source 1..25: mol_type = tRNA
gtggttagtactctgcgttgtggcc

SEQ ID NO: 592 - RNA, 26 nt, Homo sapiens
  source 1..26: mol_type = tRNA
gtggttagtactctgcgttgtggccg

SEQ ID NO: 593 - RNA, 27 nt, Homo sapiens
  source 1..27: mol_type = tRNA
gtggttagtactctgcgttgtggccgc

SEQ ID NO: 594 - RNA, 30 nt, Homo sapiens
  source 1..30: mol_type = tRNA
gtggttagtactctgcgttgtggccgcagc

SEQ ID NO: 595 - RNA, 17 nt, Homo sapiens
  source 1..17: mol_type = tRNA
gttagtactctgcgctg

SEQ ID NO: 596 - RNA, 18 nt, Homo sapiens
  source 1..18: mol_type = tRNA
gttagtactctgcgctgt

SEQ ID NO: 597 - RNA, 16 nt, Homo sapiens
  source 1..16: mol_type = tRNA
gttagtactctgcgtt

SEQ ID NO: 598 - RNA, 17 nt, Homo sapiens
  source 1..17: mol_type = tRNA
gttagtactctgcgttg

SEQ ID NO: 599 - RNA, 18 nt, Homo sapiens
  source 1..18: mol_type = tRNA
gttagtactctgcgttgt

SEQ ID NO: 600 - RNA, 19 nt, Homo sapiens
  source 1..19: mol_type = tRNA
gttagtactctgcgttgtg

SEQ ID NO: 601 - RNA, 20 nt, Homo sapiens
  source 1..20: mol_type = tRNA
gttagtactctgcgttgtgg

SEQ ID NO: 602 - RNA, 21 nt, Homo sapiens
  source 1..21: mol_type = tRNA
gttagtactctgcgttgtggc

SEQ ID NO: 603 - RNA, 22 nt, Homo sapiens
  source 1..22: mol_type = tRNA
gttagtactctgcgttgtggcc

SEQ ID NO: 604 - RNA, 23 nt, Homo sapiens
  source 1..23: mol_type = tRNA
gttagtactctgcgttgtggccg

SEQ ID NO: 605 - RNA, 24 nt, Homo sapiens
  source 1..24: mol_type = tRNA
gttagtactctgcgttgtggccgc

SEQ ID NO: 606 - RNA, 27 nt, Homo sapiens
  source 1..27: mol_type = tRNA
gttagtactctgcgttgtggccgcagc

SEQ ID NO: 607 - RNA, 17 nt, Homo sapiens
  source 1..17: mol_type = tRNA
gttcgaatccgagtcac

SEQ ID NO: 608 - RNA, 18 nt, Homo sapiens
  source 1..18: mol_type = tRNA
gttcgaatccgagtcacg

SEQ ID NO: 609 - RNA, 19 nt, Homo sapiens
  source 1..19: mol_type = tRNA
gttcgaatccgagtcacgg

SEQ ID NO: 610 - RNA, 20 nt, Homo sapiens
  source 1..20: mol_type = tRNA
gttcgaatccgagtcacggc

SEQ ID NO: 611 - RNA, 21 nt, Homo sapiens
  source 1..21: mol_type = tRNA
gttcgaatccgagtcacggca

SEQ ID NO: 612 - RNA, 20 nt, Homo sapiens
  source 1..20: mol_type = tRNA
gttgtggccgcagcaacctc

SEQ ID NO: 613 - RNA, 21 nt, Homo sapiens
  source 1..21: mol_type = tRNA
gttgtggccgcagcaacctcg

SEQ ID NO: 614 - RNA, 22 nt, Homo sapiens
  source 1..22: mol_type = tRNA
gttgtggccgcagcaacctcgg

SEQ ID NO: 615 - RNA, 23 nt, Homo sapiens
  source 1..23: mol_type = tRNA
gttgtggccgcagcaacctcggt

SEQ ID NO: 616 - RNA, 18 nt, Homo sapiens
  source 1..18: mol_type = tRNA
gtttaaccaaaacatcag

SEQ ID NO: 617 - RNA, 22 nt, Homo sapiens
  source 1..22: mol_type = tRNA
gtttaaccaaaacatcagattg

SEQ ID NO: 618 - RNA, 23 nt, Homo sapiens
  source 1..23: mol_type = tRNA
gtttaaccaaaacatcagattgt

SEQ ID NO: 619 - RNA, 24 nt, Homo sapiens
  source 1..24: mol_type = tRNA
gtttaaccaaaacatcagattgtg

SEQ ID NO: 620 - RNA, 25 nt, Homo sapiens
  source 1..25: mol_type = tRNA
gtttaaccaaaacatcagattgtga

SEQ ID NO: 621 - RNA, 27 nt, Homo sapiens
  source 1..27: mol_type = tRNA
gtttaaccaaaacatcagattgtgaat

SEQ ID NO: 622 - RNA, 28 nt, Homo sapiens
  source 1..28: mol_type = tRNA
gtttaaccaaaacatcagattgtgaatc

SEQ ID NO: 623 - RNA, 29 nt, Homo sapiens
  source 1..29: mol_type = tRNA
gtttaaccaaaacatcagattgtgaatct

SEQ ID NO: 624 - RNA, 30 nt, Homo sapiens
  source 1..30: mol_type = tRNA
gtttaaccaaaacatcagattgtgaatctg

SEQ ID NO: 625 - RNA, 30 nt, Homo sapiens
  source 1..30: mol_type = tRNA
taaatatagtttaaccaaaacatcagattg

SEQ ID NO: 626 - RNA, 19 nt, Homo sapiens
  source 1..19: mol_type = tRNA
taaccaaaacatcagattg

SEQ ID NO: 627 - RNA, 20 nt, Homo sapiens
  source 1..20: mol_type = tRNA
taaccaaaacatcagattgt

SEQ ID NO: 628 - RNA, 21 nt, Homo sapiens
  source 1..21: mol_type = tRNA
taaccaaaacatcagattgtg

SEQ ID NO: 629 - RNA, 22 nt, Homo sapiens
  source 1..22: mol_type = tRNA
taaccaaaacatcagattgtga

SEQ ID NO: 630 - RNA, 23 nt, Homo sapiens
  source 1..23: mol_type = tRNA
taaccaaaacatcagattgtgaa

SEQ ID NO: 631 - RNA, 24 nt, Homo sapiens
  source 1..24: mol_type = tRNA
taaccaaaacatcagattgtgaat

SEQ ID NO: 632 - RNA, 25 nt, Homo sapiens
  source 1..25: mol_type = tRNA
taaccaaaacatcagattgtgaatc

SEQ ID NO: 633 - RNA, 26 nt, Homo sapiens
  source 1..26: mol_type = tRNA
taaccaaaacatcagattgtgaatct

SEQ ID NO: 634 - RNA, 27 nt, Homo sapiens
  source 1..27: mol_type = tRNA
taaccaaaacatcagattgtgaatctg

SEQ ID NO: 635 - RNA, 29 nt, Homo sapiens
  source 1..29: mol_type = tRNA
taaccaaaacatcagattgtgaatctgac

SEQ ID NO: 636 - RNA, 18 nt, Homo sapiens
  source 1..18: mol_type = tRNA
tacgaccccttatttacc

SEQ ID NO: 637 - RNA, 16 nt, Homo sapiens
  source 1..16: mol_type = tRNA
tactctgcgttgtggc

SEQ ID NO: 638 - RNA, 17 nt, Homo sapiens
  source 1..17: mol_type = tRNA
tactctgcgttgtggcc

SEQ ID NO: 639 - RNA, 18 nt, Homo sapiens
  source 1..18: mol_type = tRNA
tactctgcgttgtggccg

SEQ ID NO: 640 - RNA, 19 nt, Homo sapiens
  source 1..19: mol_type = tRNA
tactctgcgttgtggccgc

SEQ ID NO: 641 - RNA, 21 nt, Homo sapiens
  source 1..21: mol_type = tRNA
tactctgcgttgtggccgcag

SEQ ID NO: 642 - RNA, 22 nt, Homo sapiens
  source 1..22: mol_type = tRNA
tactctgcgttgtggccgcagc

SEQ ID NO: 643 - RNA, 16 nt, Homo sapiens
  source 1..16: mol_type = tRNA
tagtactctgcgttgt

SEQ ID NO: 644 - RNA, 17 nt, Homo sapiens
  source 1..17: mol_type = tRNA
tagtactctgcgttgtg

SEQ ID NO: 645 - RNA, 18 nt, Homo sapiens
  source 1..18: mol_type = tRNA
tagtactctgcgttgtgg

SEQ ID NO: 646 - RNA, 19 nt, Homo sapiens
  source 1..19: mol_type = tRNA
tagtactctgcgttgtggc

SEQ ID NO: 647 - RNA, 20 nt, Homo sapiens
  source 1..20: mol_type = tRNA
tagtactctgcgttgtggcc

SEQ ID NO: 648 - RNA, 21 nt, Homo sapiens
  source 1..21: mol_type = tRNA
tagtactctgcgttgtggccg

SEQ ID NO: 649 - RNA, 22 nt, Homo sapiens
  source 1..22: mol_type = tRNA
tagtactctgcgttgtggccgc

SEQ ID NO: 650 - RNA, 23 nt, Homo sapiens
  source 1..23: mol_type = tRNA
tagtactctgcgttgtggccgca

SEQ ID NO: 651 - RNA, 25 nt, Homo sapiens
  source 1..25: mol_type = tRNA
tagtactctgcgttgtggccgcagc

SEQ ID NO: 652 - RNA, 30 nt, Homo sapiens
  source 1..30: mol_type = tRNA
tagtactctgcgttgtggccgcagcaacct

SEQ ID NO: 653 - RNA, 16 nt, Homo sapiens
  source 1..16: mol_type = tRNA
tagtggttagtactct

SEQ ID NO: 654 - RNA, 17 nt, Homo sapiens
  source 1..17: mol_type = tRNA
tagtggttagtactctg

SEQ ID NO: 655 - RNA, 18 nt, Homo sapiens
  source 1..18: mol_type = tRNA
tagtggttagtactctgc

SEQ ID NO: 656 - RNA, 19 nt, Homo sapiens
  source 1..19: mol_type = tRNA
tagtggttagtactctgcg

SEQ ID NO: 657 - RNA, 20 nt, Homo sapiens
  source 1..20: mol_type = tRNA
tagtggttagtactctgcgc

SEQ ID NO: 658 - RNA, 21 nt, Homo sapiens
  source 1..21: mol_type = tRNA
tagtggttagtactctgcgct

SEQ ID NO: 659 - RNA, 22 nt, Homo sapiens
  source 1..22: mol_type = tRNA
tagtggttagtactctgcgctg

SEQ ID NO: 660 - RNA, 23 nt, Homo sapiens
  source 1..23: mol_type = tRNA
tagtggttagtactctgcgctgt

SEQ ID NO: 661 - RNA, 20 nt, Homo sapiens
  source 1..20: mol_type = tRNA
tagtggttagtactctgcgt

SEQ ID NO: 662 - RNA, 21 nt, Homo sapiens
  source 1..21: mol_type = tRNA
tagtggttagtactctgcgtt

SEQ ID NO: 663 - RNA, 22 nt, Homo sapiens
  source 1..22: mol_type = tRNA
tagtggttagtactctgcgttg

SEQ ID NO: 664 - RNA, 23 nt, Homo sapiens
  source 1..23: mol_type = tRNA
tagtggttagtactctgcgttgt

SEQ ID NO: 665 - RNA, 24 nt, Homo sapiens
  source 1..24: mol_type = tRNA
tagtggttagtactctgcgttgtg

SEQ ID NO: 666 - RNA, 25 nt, Homo sapiens
  source 1..25: mol_type = tRNA
tagtggttagtactctgcgttgtgg

SEQ ID NO: 667 - RNA, 26 nt, Homo sapiens
  source 1..26: mol_type = tRNA
tagtggttagtactctgcgttgtggc

SEQ ID NO: 668 - RNA, 27 nt, Homo sapiens
  source 1..27: mol_type = tRNA
tagtggttagtactctgcgttgtggcc

SEQ ID NO: 669 - RNA, 28 nt, Homo sapiens
  source 1..28: mol_type = tRNA
tagtggttagtactctgcgttgtggccg

SEQ ID NO: 670 - RNA, 29 nt, Homo sapiens
  source 1..29: mol_type = tRNA
tagtggttagtactctgcgttgtggccgc

SEQ ID NO: 671 - RNA, 26 nt, Homo sapiens
  source 1..26: mol_type = tRNA
tagtttaaccaaaacatcagattgtg

SEQ ID NO: 672 - RNA, 27 nt, Homo sapiens
  source 1..27: mol_type = tRNA
tagtttaaccaaaacatcagattgtga

SEQ ID NO: 673 - RNA, 16 nt, Homo sapiens
  source 1..16: mol_type = tRNA
tatagtggttagtact

SEQ ID NO: 674 - RNA, 18 nt, Homo sapiens
  source 1..18: mol_type = tRNA
tatagtggttagtactct

SEQ ID NO: 675 - RNA, 19 nt, Homo sapiens
  source 1..19: mol_type = tRNA
tatagtggttagtactctg

SEQ ID NO: 676 - RNA, 20 nt, Homo sapiens
  source 1..20: mol_type = tRNA
tatagtggttagtactctgc

SEQ ID NO: 677 - RNA, 21 nt, Homo sapiens
  source 1..21: mol_type = tRNA
tatagtggttagtactctgcg

SEQ ID NO: 678 - RNA, 22 nt, Homo sapiens
  source 1..22: mol_type = tRNA
tatagtggttagtactctgcgc

SEQ ID NO: 679 - RNA, 23 nt, Homo sapiens
  source 1..23: mol_type = tRNA
tatagtggttagtactctgcgct

SEQ ID NO: 680 - RNA, 24 nt, Homo sapiens
  source 1..24: mol_type = tRNA
tatagtggttagtactctgcgctg

SEQ ID NO: 681 - RNA, 22 nt, Homo sapiens
  source 1..22: mol_type = tRNA
tatagtggttagtactctgcgt

SEQ ID NO: 682 - RNA, 23 nt, Homo sapiens
  source 1..23: mol_type = tRNA
tatagtggttagtactctgcgtt

SEQ ID NO: 683 - RNA, 24 nt, Homo sapiens
  source 1..24: mol_type = tRNA
tatagtggttagtactctgcgttg

SEQ ID NO: 684 - RNA, 25 nt, Homo sapiens
  source 1..25: mol_type = tRNA
tatagtggttagtactctgcgttgt

SEQ ID NO: 685 - RNA, 26 nt, Homo sapiens
  source 1..26: mol_type = tRNA
tatagtggttagtactctgcgttgtg

SEQ ID NO: 686 - RNA, 27 nt, Homo sapiens
  source 1..27: mol_type = tRNA
tatagtggttagtactctgcgttgtgg

SEQ ID NO: 687 - RNA, 28 nt, Homo sapiens
  source 1..28: mol_type = tRNA
tatagtggttagtactctgcgttgtggc

SEQ ID NO: 688 - RNA, 29 nt, Homo sapiens
  source 1..29: mol_type = tRNA
tatagtggttagtactctgcgttgtggcc

SEQ ID NO: 689 - RNA, 30 nt, Homo sapiens
  source 1..30: mol_type = tRNA
tatagtggttagtactctgcgttgtggccg

SEQ ID NO: 690 - RNA, 29 nt, Homo sapiens
  source 1..29: mol_type = tRNA
tatagtttaaccaaaacatcagattgtga

SEQ ID NO: 691 - RNA, 16 nt, Homo sapiens
  source 1..16: mol_type = tRNA
tcagattgtgaatctg

SEQ ID NO: 692 - RNA, 17 nt, Homo sapiens
  source 1..17: mol_type = tRNA
tcagattgtgaatctga

SEQ ID NO: 693 - RNA, 18 nt, Homo sapiens
  source 1..18: mol_type = tRNA
tcagattgtgaatctgac

SEQ ID NO: 694 - RNA, 19 nt, Homo sapiens
  source 1..19: mol_type = tRNA
tcagattgtgaatctgaca

SEQ ID NO: 695 - RNA, 21 nt, Homo sapiens
  source 1..21: mol_type = tRNA
tcagattgtgaatctgacaac

SEQ ID NO: 696 - RNA, 22 nt, Homo sapiens
  source 1..22: mol_type = tRNA
tcagattgtgaatctgacaaca

SEQ ID NO: 697 - RNA, 23 nt, Homo sapiens
  source 1..23: mol_type = tRNA
tcagattgtgaatctgacaacag

SEQ ID NO: 698 - RNA, 24 nt, Homo sapiens
  source 1..24: mol_type = tRNA
tcagattgtgaatctgacaacaga

SEQ ID NO: 699 - RNA, 25 nt, Homo sapiens
  source 1..25: mol_type = tRNA
tcagattgtgaatctgacaacagag

SEQ ID NO: 700 - RNA, 26 nt, Homo sapiens
  source 1..26: mol_type = tRNA
tcagattgtgaatctgacaacagagg

SEQ ID NO: 701 - RNA, 27 nt, Homo sapiens
  source 1..27: mol_type = tRNA
tcagattgtgaatctgacaacagaggc

SEQ ID NO: 702 - RNA, 28 nt, Homo sapiens
  source 1..28: mol_type = tRNA
tcagattgtgaatctgacaacagaggct

SEQ ID NO: 703 - RNA, 29 nt, Homo sapiens
  source 1..29: mol_type = tRNA
tcagattgtgaatctgacaacagaggctt

SEQ ID NO: 704 - RNA, 19 nt, Homo sapiens
  source 1..19: mol_type = tRNA
tcgaatccgagtcacggca

SEQ ID NO: 705 - RNA, 16 nt, Homo sapiens
  source 1..16: mol_type = tRNA
tcgtatagtggttagt

SEQ ID NO: 706 - RNA, 17 nt, Homo sapiens
  source 1..17: mol_type = tRNA
tcgtatagtggttagta

SEQ ID NO: 707 - RNA, 19 nt, Homo sapiens
  source 1..19: mol_type = tRNA
tcgtatagtggttagtact

SEQ ID NO: 708 - RNA, 20 nt, Homo sapiens
  source 1..20: mol_type = tRNA
tcgtatagtggttagtactc

SEQ ID NO: 709 - RNA, 21 nt, Homo sapiens
  source 1..21: mol_type = tRNA
tcgtatagtggttagtactct

SEQ ID NO: 710 - RNA, 22 nt, Homo sapiens
  source 1..22: mol_type = tRNA
tcgtatagtggttagtactctg

SEQ ID NO: 711 - RNA, 23 nt, Homo sapiens
  source 1..23: mol_type = tRNA
tcgtatagtggttagtactctgc

SEQ ID NO: 712 - RNA, 24 nt, Homo sapiens
  source 1..24: mol_type = tRNA
tcgtatagtggttagtactctgcg

SEQ ID NO: 713 - RNA, 25 nt, Homo sapiens
  source 1..25: mol_type = tRNA
tcgtatagtggttagtactctgcgc

SEQ ID NO: 714 - RNA, 25 nt, Homo sapiens
  source 1..25: mol_type = tRNA
tcgtatagtggttagtactctgcgt

SEQ ID NO: 715 - RNA, 26 nt, Homo sapiens
  source 1..26: mol_type = tRNA
tcgtatagtggttagtactctgcgtt

SEQ ID NO: 716 - RNA, 27 nt, Homo sapiens
  source 1..27: mol_type = tRNA
tcgtatagtggttagtactctgcgttg

SEQ ID NO: 717 - RNA, 28 nt, Homo sapiens
  source 1..28: mol_type = tRNA
tcgtatagtggttagtactctgcgttgt

SEQ ID NO: 718 - RNA, 29 nt, Homo sapiens
  source 1..29: mol_type = tRNA
tcgtatagtggttagtactctgcgttgtg

SEQ ID NO: 719 - RNA, 30 nt, Homo sapiens
  source 1..30: mol_type = tRNA
tcgtatagtggttagtactctgcgttgtgg

SEQ ID NO: 720 - RNA, 22 nt, Homo sapiens
  source 1..22: mol_type = tRNA
tctgacaacagaggcttacgac

SEQ ID NO: 721 - RNA, 25 nt, Homo sapiens
  source 1..25: mol_type = tRNA
tctgacaacagaggcttacgacccc

SEQ ID NO: 722 - RNA, 26 nt, Homo sapiens
  source 1..26: mol_type = tRNA
tctgacaacagaggcttacgacccct

SEQ ID NO: 723 - RNA, 27 nt, Homo sapiens
  source 1..27: mol_type = tRNA
tctgacaacagaggcttacgacccctt

SEQ ID NO: 724 - RNA, 28 nt, Homo sapiens
  source 1..28: mol_type = tRNA
tctgacaacagaggcttacgacccctta

SEQ ID NO: 725 - RNA, 30 nt, Homo sapiens
  source 1..30: mol_type = tRNA
tctgacaacagaggcttacgaccccttatt

SEQ ID NO: 726 - RNA, 17 nt, Homo sapiens
  source 1..17: mol_type = tRNA
tgaatctgacaacagag

SEQ ID NO: 727 - RNA, 18 nt, Homo sapiens
  source 1..18: mol_type = tRNA
tgaatctgacaacagagg

SEQ ID NO: 728 - RNA, 19 nt, Homo sapiens
  source 1..19: mol_type = tRNA
tgaatctgacaacagaggc

SEQ ID NO: 729 - RNA, 20 nt, Homo sapiens
  source 1..20: mol_type = tRNA
tgaatctgacaacagaggct

SEQ ID NO: 730 - RNA, 21 nt, Homo sapiens
  source 1..21: mol_type = tRNA
tgaatctgacaacagaggctt

SEQ ID NO: 731 - RNA, 22 nt, Homo sapiens
  source 1..22: mol_type = tRNA
tgaatctgacaacagaggctta

SEQ ID NO: 732 - RNA, 23 nt, Homo sapiens
  source 1..23: mol_type = tRNA
tgaatctgacaacagaggcttac

SEQ ID NO: 733 - RNA, 24 nt, Homo sapiens
  source 1..24: mol_type = tRNA
tgaatctgacaacagaggcttacg

SEQ ID NO: 734 - RNA, 25 nt, Homo sapiens
  source 1..25: mol_type = tRNA
tgaatctgacaacagaggcttacga

SEQ ID NO: 735 - RNA, 26 nt, Homo sapiens
  source 1..26: mol_type = tRNA
tgaatctgacaacagaggcttacgac

SEQ ID NO: 736 - RNA, 27 nt, Homo sapiens
  source 1..27: mol_type = tRNA
tgaatctgacaacagaggcttacgacc

SEQ ID NO: 737 - RNA, 28 nt, Homo sapiens
  source 1..28: mol_type = tRNA
tgaatctgacaacagaggcttacgaccc

SEQ ID NO: 738 - RNA, 29 nt, Homo sapiens
  source 1..29: mol_type = tRNA
tgaatctgacaacagaggcttacgacccc

SEQ ID NO: 739 - RNA, 30 nt, Homo sapiens
  source 1..30: mol_type = tRNA
tgaatctgacaacagaggcttacgacccct

SEQ ID NO: 740 - RNA, 18 nt, Homo sapiens
  source 1..18: mol_type = tRNA
tgatcgtatagtggttag

SEQ ID NO: 741 - RNA, 19 nt, Homo sapiens
  source 1..19: mol_type = tRNA
tgatcgtatagtggttagt

SEQ ID NO: 742 - RNA, 20 nt, Homo sapiens
  source 1..20: mol_type = tRNA
tgatcgtatagtggttagta

SEQ ID NO: 743 - RNA, 21 nt, Homo sapiens
  source 1..21: mol_type = tRNA
tgatcgtatagtggttagtac

SEQ ID NO: 744 - RNA, 22 nt, Homo sapiens
  source 1..22: mol_type = tRNA
tgatcgtatagtggttagtact

SEQ ID NO: 745 - RNA, 23 nt, Homo sapiens
  source 1..23: mol_type = tRNA
tgatcgtatagtggttagtactc

SEQ ID NO: 746 - RNA, 24 nt, Homo sapiens
  source 1..24: mol_type = tRNA
tgatcgtatagtggttagtactct

SEQ ID NO: 747 - RNA, 25 nt, Homo sapiens
  source 1..25: mol_type = tRNA
tgatcgtatagtggttagtactctg

SEQ ID NO: 748 - RNA, 26 nt, Homo sapiens
  source 1..26: mol_type = tRNA
tgatcgtatagtggttagtactctgc

SEQ ID NO: 749 - RNA, 27 nt, Homo sapiens
  source 1..27: mol_type = tRNA
tgatcgtatagtggttagtactctgcg

SEQ ID NO: 750 - RNA, 28 nt, Homo sapiens
  source 1..28: mol_type = tRNA
tgatcgtatagtggttagtactctgcgc

SEQ ID NO: 751 - RNA, 28 nt, Homo sapiens
  source 1..28: mol_type = tRNA
tgatcgtatagtggttagtactctgcgt

SEQ ID NO: 752 - RNA, 29 nt, Homo sapiens
  source 1..29: mol_type = tRNA
tgatcgtatagtggttagtactctgcgtt

SEQ ID NO: 753 - RNA, 30 nt, Homo sapiens
  source 1..30: mol_type = tRNA
tgatcgtatagtggttagtactctgcgttg

SEQ ID NO: 754 - RNA, 21 nt, Homo sapiens
  source 1..21: mol_type = tRNA
tgcgttgtggccgcagcaacc

SEQ ID NO: 755 - RNA, 16 nt, Homo sapiens
  source 1..16: mol_type = tRNA
tggccgcagcaacctc

SEQ ID NO: 756 - RNA, 17 nt, Homo sapiens
  source 1..17: mol_type = tRNA
tggccgcagcaacctcg

SEQ ID NO: 757 - RNA, 18 nt, Homo sapiens
  source 1..18: mol_type = tRNA
tggccgcagcaacctcgg

SEQ ID NO: 758 - RNA, 19 nt, Homo sapiens
  source 1..19: mol_type = tRNA
tggccgcagcaacctcggt

SEQ ID NO: 759 - RNA, 21 nt, Homo sapiens
  source 1..21: mol_type = tRNA
tggccgcagcaacctcggttc

SEQ ID NO: 760 - RNA, 22 nt, Homo sapiens
  source 1..22: mol_type = tRNA
tggccgcagcaacctcggttcg

SEQ ID NO: 761 - RNA, 23 nt, Homo sapiens
  source 1..23: mol_type = tRNA
tggccgcagcaacctcggttcga

SEQ ID NO: 762 - RNA, 24 nt, Homo sapiens
  source 1..24: mol_type = tRNA
tggccgcagcaacctcggttcgaa

SEQ ID NO: 763 - RNA, 16 nt, Homo sapiens
  source 1..16: mol_type = tRNA
tggttagtactctgcg

SEQ ID NO: 764 - RNA, 17 nt, Homo sapiens
  source 1..17: mol_type = tRNA
tggttagtactctgcgc

SEQ ID NO: 765 - RNA, 18 nt, Homo sapiens
  source 1..18: mol_type = tRNA
tggttagtactctgcgct

SEQ ID NO: 766 - RNA, 19 nt, Homo sapiens
  source 1..19: mol_type = tRNA
tggttagtactctgcgctg

SEQ ID NO: 767 - RNA, 20 nt, Homo sapiens
  source 1..20: mol_type = tRNA
tggttagtactctgcgctgt

SEQ ID NO: 768 - RNA, 17 nt, Homo sapiens
  source 1..17: mol_type = tRNA
tggttagtactctgcgt

SEQ ID NO: 769 - RNA, 18 nt, Homo sapiens
  source 1..18: mol_type = tRNA
tggttagtactctgcgtt

SEQ ID NO: 770 - RNA, 19 nt, Homo sapiens
  source 1..19: mol_type = tRNA
tggttagtactctgcgttg

SEQ ID NO: 771 - RNA, 20 nt, Homo sapiens
  source 1..20: mol_type = tRNA
tggttagtactctgcgttgt

SEQ ID NO: 772 - RNA, 21 nt, Homo sapiens
  source 1..21: mol_type = tRNA
tggttagtactctgcgttgtg

SEQ ID NO: 773 - RNA, 22 nt, Homo sapiens
  source 1..22: mol_type = tRNA
tggttagtactctgcgttgtgg

SEQ ID NO: 774 - RNA, 23 nt, Homo sapiens
  source 1..23: mol_type = tRNA
tggttagtactctgcgttgtggc

SEQ ID NO: 775 - RNA, 24 nt, Homo sapiens
  source 1..24: mol_type = tRNA
tggttagtactctgcgttgtggcc

SEQ ID NO: 776 - RNA, 25 nt, Homo sapiens
  source 1..25: mol_type = tRNA
tggttagtactctgcgttgtggccg

SEQ ID NO: 777 - RNA, 26 nt, Homo sapiens
  source 1..26: mol_type = tRNA
tggttagtactctgcgttgtggccgc

SEQ ID NO: 778 - RNA, 27 nt, Homo sapiens
  source 1..27: mol_type = tRNA
tggttagtactctgcgttgtggccgca

SEQ ID NO: 779 - RNA, 29 nt, Homo sapiens
  source 1..29: mol_type = tRNA
tggttagtactctgcgttgtggccgcagc

SEQ ID NO: 780 - RNA, 16 nt, Homo sapiens
  source 1..16: mol_type = tRNA
tgtgaatctgacaaca

SEQ ID NO: 781 - RNA, 17 nt, Homo sapiens
  source 1..17: mol_type = tRNA
tgtgaatctgacaacag

SEQ ID NO: 782 - RNA, 18 nt, Homo sapiens
  source 1..18: mol_type = tRNA
tgtgaatctgacaacaga

SEQ ID NO: 783 - RNA, 19 nt, Homo sapiens
  source 1..19: mol_type = tRNA
tgtgaatctgacaacagag

SEQ ID NO: 784 - RNA, 20 nt, Homo sapiens
  source 1..20: mol_type = tRNA
tgtgaatctgacaacagagg

SEQ ID NO: 785 - RNA, 21 nt, Homo sapiens
  source 1..21: mol_type = tRNA
tgtgaatctgacaacagaggc

SEQ ID NO: 786 - RNA, 22 nt, Homo sapiens
  source 1..22: mol_type = tRNA
tgtgaatctgacaacagaggct

SEQ ID NO: 787 - RNA, 23 nt, Homo sapiens
  source 1..23: mol_type = tRNA
tgtgaatctgacaacagaggctt

SEQ ID NO: 788 - RNA, 24 nt, Homo sapiens
  source 1..24: mol_type = tRNA
tgtgaatctgacaacagaggctta

SEQ ID NO: 789 - RNA, 25 nt, Homo sapiens
  source 1..25: mol_type = tRNA
tgtgaatctgacaacagaggcttac

SEQ ID NO: 790 - RNA, 26 nt, Homo sapiens
  source 1..26: mol_type = tRNA
tgtgaatctgacaacagaggcttacg

SEQ ID NO: 791 - RNA, 27 nt, Homo sapiens
  source 1..27: mol_type = tRNA
tgtgaatctgacaacagaggcttacga

SEQ ID NO: 792 - RNA, 28 nt, Homo sapiens
  source 1..28: mol_type = tRNA
tgtgaatctgacaacagaggcttacgac

SEQ ID NO: 793 - RNA, 29 nt, Homo sapiens
  source 1..29: mol_type = tRNA
tgtgaatctgacaacagaggcttacgacc

SEQ ID NO: 794 - RNA, 30 nt, Homo sapiens
  source 1..30: mol_type = tRNA
tgtgaatctgacaacagaggcttacgaccc

SEQ ID NO: 795 - RNA, 18 nt, Homo sapiens
  source 1..18: mol_type = tRNA
tgtggccgcagcaacctc

SEQ ID NO: 796 - RNA, 20 nt, Homo sapiens
  source 1..20: mol_type = tRNA
tgtggccgcagcaacctcgg

SEQ ID NO: 797 - RNA, 21 nt, Homo sapiens
  source 1..21: mol_type = tRNA
tgtggccgcagcaacctcggt

SEQ ID NO: 798 - RNA, 16 nt, Homo sapiens
  source 1..16: mol_type = tRNA
ttaaccaaaacatcag

SEQ ID NO: 799 - RNA, 17 nt, Homo sapiens
  source 1..17: mol_type = tRNA
ttaaccaaaacatcaga

SEQ ID NO: 800 - RNA, 20 nt, Homo sapiens
  source 1..20: mol_type = tRNA
ttaaccaaaacatcagattg

SEQ ID NO: 801 - RNA, 21 nt, Homo sapiens
  source 1..21: mol_type = tRNA
ttaaccaaaacatcagattgt

SEQ ID NO: 802 - RNA, 22 nt, Homo sapiens
  source 1..22: mol_type = tRNA
ttaaccaaaacatcagattgtg

SEQ ID NO: 803 - RNA, 23 nt, Homo sapiens
  source 1..23: mol_type = tRNA
ttaaccaaaacatcagattgtga

SEQ ID NO: 804 - RNA, 24 nt, Homo sapiens
  source 1..24: mol_type = tRNA
ttaaccaaaacatcagattgtgaa

SEQ ID NO: 805 - RNA, 25 nt, Homo sapiens
  source 1..25: mol_type = tRNA
ttaaccaaaacatcagattgtgaat

SEQ ID NO: 806 - RNA, 26 nt, Homo sapiens
  source 1..26: mol_type = tRNA
ttaaccaaaacatcagattgtgaatc

SEQ ID NO: 807 - RNA, 27 nt, Homo sapiens
  source 1..27: mol_type = tRNA
ttaaccaaaacatcagattgtgaatct

SEQ ID NO: 808 - RNA, 29 nt, Homo sapiens
  source 1..29: mol_type = tRNA
ttaaccaaaacatcagattgtgaatctga

SEQ ID NO: 809 - RNA, 30 nt, Homo sapiens
  source 1..30: mol_type = tRNA
ttaaccaaaacatcagattgtgaatctgac

SEQ ID NO: 810 - RNA, 17 nt, Homo sapiens
  source 1..17: mol_type = tRNA
ttacgaccccttattta

SEQ ID NO: 811 - RNA, 18 nt, Homo sapiens
  source 1..18: mol_type = tRNA
ttacgaccccttatttac

SEQ ID NO: 812 - RNA, 19 nt, Homo sapiens
  source 1..19: mol_type = tRNA
ttacgaccccttatttacc

SEQ ID NO: 813 - RNA, 16 nt, Homo sapiens
  source 1..16: mol_type = tRNA
ttagtactctgcgttg

SEQ ID NO: 814 - RNA, 17 nt, Homo sapiens
  source 1..17: mol_type = tRNA
ttagtactctgcgttgt

SEQ ID NO: 815 - RNA, 18 nt, Homo sapiens
  source 1..18: mol_type = tRNA
ttagtactctgcgttgtg

SEQ ID NO: 816 - RNA, 19 nt, Homo sapiens
  source 1..19: mol_type = tRNA
ttagtactctgcgttgtgg

SEQ ID NO: 817 - RNA, 20 nt, Homo sapiens
  source 1..20: mol_type = tRNA
ttagtactctgcgttgtggc

SEQ ID NO: 818 - RNA, 21 nt, Homo sapiens
  source 1..21: mol_type = tRNA
ttagtactctgcgttgtggcc

SEQ ID NO: 819 - RNA, 22 nt, Homo sapiens
  source 1..22: mol_type = tRNA
ttagtactctgcgttgtggccg

SEQ ID NO: 820 - RNA, 23 nt, Homo sapiens
  source 1..23: mol_type = tRNA
ttagtactctgcgttgtggccgc

SEQ ID NO: 821 - RNA, 24 nt, Homo sapiens
  source 1..24: mol_type = tRNA
ttagtactctgcgttgtggccgca

SEQ ID NO: 822 - RNA, 26 nt, Homo sapiens
  source 1..26: mol_type = tRNA
ttagtactctgcgttgtggccgcagc

SEQ ID NO: 823 - RNA, 30 nt, Homo sapiens
  source 1..30: mol_type = tRNA
ttagtactctgcgttgtggccgcagcaacc

SEQ ID NO: 824 - RNA, 19 nt, Homo sapiens
  source 1..19: mol_type = tRNA
ttcgaatccgagtcacggc

SEQ ID NO: 825 - RNA, 20 nt, Homo sapiens
  source 1..20: mol_type = tRNA
ttcgaatccgagtcacggca

SEQ ID NO: 826 - RNA, 16 nt, Homo sapiens
  source 1..16: mol_type = tRNA
ttgtgaatctgacaac

SEQ ID NO: 827 - RNA, 17 nt, Homo sapiens
  source 1..17: mol_type = tRNA
ttgtgaatctgacaaca

SEQ ID NO: 828 - RNA, 18 nt, Homo sapiens
  source 1..18: mol_type = tRNA
ttgtgaatctgacaacag

SEQ ID NO: 829 - RNA, 19 nt, Homo sapiens
  source 1..19: mol_type = tRNA
ttgtgaatctgacaacaga

SEQ ID NO: 830 - RNA, 20 nt, Homo sapiens
  source 1..20: mol_type = tRNA
ttgtgaatctgacaacagag

SEQ ID NO: 831 - RNA, 21 nt, Homo sapiens
  source 1..21: mol_type = tRNA
ttgtgaatctgacaacagagg

SEQ ID NO: 832 - RNA, 22 nt, Homo sapiens
  source 1..22: mol_type = tRNA
ttgtgaatctgacaacagaggc

SEQ ID NO: 833 - RNA, 23 nt, Homo sapiens
  source 1..23: mol_type = tRNA
ttgtgaatctgacaacagaggct

SEQ ID NO: 834 - RNA, 24 nt, Homo sapiens
  source 1..24: mol_type = tRNA
ttgtgaatctgacaacagaggctt

SEQ ID NO: 835 - RNA, 25 nt, Homo sapiens
  source 1..25: mol_type = tRNA
ttgtgaatctgacaacagaggctta

SEQ ID NO: 836 - RNA, 26 nt, Homo sapiens
  source 1..26: mol_type = tRNA
ttgtgaatctgacaacagaggcttac

SEQ ID NO: 837 - RNA, 27 nt, Homo sapiens
  source 1..27: mol_type = tRNA
ttgtgaatctgacaacagaggcttacg

SEQ ID NO: 838 - RNA, 28 nt, Homo sapiens
  source 1..28: mol_type = tRNA
ttgtgaatctgacaacagaggcttacga

SEQ ID NO: 839 - RNA, 29 nt, Homo sapiens
  source 1..29: mol_type = tRNA
ttgtgaatctgacaacagaggcttacgac

SEQ ID NO: 840 - RNA, 30 nt, Homo sapiens
  source 1..30: mol_type = tRNA
ttgtgaatctgacaacagaggcttacgacc

SEQ ID NO: 841 - RNA, 17 nt, Homo sapiens
  source 1..17: mol_type = tRNA
ttgtggccgcagcaacc

SEQ ID NO: 842 - RNA, 18 nt, Homo sapiens
  source 1..18: mol_type = tRNA
ttgtggccgcagcaacct

SEQ ID NO: 843 - RNA, 19 nt, Homo sapiens
  source 1..19: mol_type = tRNA
ttgtggccgcagcaacctc

SEQ ID NO: 844 - RNA, 20 nt, Homo sapiens
  source 1..20: mol_type = tRNA
ttgtggccgcagcaacctcg

SEQ ID NO: 845 - RNA, 21 nt, Homo sapiens
  source 1..21: mol_type = tRNA
ttgtggccgcagcaacctcgg

SEQ ID NO: 846 - RNA, 22 nt, Homo sapiens
  source 1..22: mol_type = tRNA
ttgtggccgcagcaacctcggt

SEQ ID NO: 847 - RNA, 23 nt, Homo sapiens
  source 1..23: mol_type = tRNA
ttgtggccgcagcaacctcggtt

SEQ ID NO: 848 - RNA, 24 nt, Homo sapiens
  source 1..24: mol_type = tRNA
ttgtggccgcagcaacctcggttc

SEQ ID NO: 849 - RNA, 17 nt, Homo sapiens
  source 1..17: mol_type = tRNA
tttaaccaaaacatcag

SEQ ID NO: 850 - RNA, 21 nt, Homo sapiens
  source 1..21: mol_type = tRNA
tttaaccaaaacatcagattg

SEQ ID NO: 851 - RNA, 22 nt, Homo sapiens
  source 1..22: mol_type = tRNA
tttaaccaaaacatcagattgt

SEQ ID NO: 852 - RNA, 23 nt, Homo sapiens
  source 1..23: mol_type = tRNA
tttaaccaaaacatcagattgtg

SEQ ID NO: 853 - RNA, 24 nt, Homo sapiens
  source 1..24: mol_type = tRNA
tttaaccaaaacatcagattgtga

SEQ ID NO: 854 - RNA, 25 nt, Homo sapiens
  source 1..25: mol_type = tRNA
tttaaccaaaacatcagattgtgaa

SEQ ID NO: 855 - RNA, 26 nt, Homo sapiens
  source 1..26: mol_type = tRNA
tttaaccaaaacatcagattgtgaat

SEQ ID NO: 856 - RNA, 27 nt, Homo sapiens
  source 1..27: mol_type = tRNA
tttaaccaaaacatcagattgtgaatc

SEQ ID NO: 857 - RNA, 28 nt, Homo sapiens
  source 1..28: mol_type = tRNA
tttaaccaaaacatcagattgtgaatct

SEQ ID NO: 858 - RNA, 29 nt, Homo sapiens
  source 1..29: mol_type = tRNA
tttaaccaaaacatcagattgtgaatctg

SEQ ID NO: 859 - RNA, 74 nt, Homo sapiens
  source 1..74: mol_type = tRNA
tcctcgtagtatagtggtgagtatccccgcctgtcacgcgggagaccggggttcgattccccgacggggagcca

SEQ ID NO: 860 - DNA, 45 bp, Homo sapiens
  source 1..45: mol_type = genomic DNA
gccgtgatcgtatagtggttagtactctgcgttgtggccgcagca

SEQ ID NO: 861 - RNA, 33 nt, Homo sapiens
  source 1..33: mol_type = tRNA
gccgtgatcgtatagtggttagtactctgcgtt

SEQ ID NO: 862 - RNA, 34 nt, Homo sapiens
  source 1..34: mol_type = tRNA
gccgtgatcgtatagtggttagtactctgcgttg

SEQ ID NO: 863 - RNA, 33 nt, Homo sapiens
  source 1..33: mol_type = tRNA
ccgtgatcgtatagtggttagtactctgcgttg

SEQ ID NO: 864 - RNA, 34 nt, Homo sapiens
  source 1..34: mol_type = tRNA
ggccgtgatcgtatagtggttagtactctgcgtt

SEQ ID NO: 865 - RNA, 35 nt, Homo sapiens
  source 1..35: mol_type = tRNA
ggccgtgatcgtatagtggttagtactctgcgttg

SEQ ID NO: 866 - RNA, 34 nt, Homo sapiens; identical to SEQ ID NO: 862
  source 1..34: mol_type = tRNA

SEQ ID NO: 867 - RNA, 34 nt, Homo sapiens
  source 1..34: mol_type = tRNA
tgccgtgatcgtatagtggttagtactctgcgtt

SEQ ID NO: 868 - RNA, 35 nt, Homo sapiens
  source 1..35: mol_type = tRNA
tgccgtgatcgtatagtggttagtactctgcgttg

SEQ ID NO: 869 - RNA, 34 nt, Homo sapiens
  source 1..34: mol_type = tRNA
tccgtgatcgtatagtggttagtactctgcgttg

SEQ ID NO: 870 - RNA, 34 nt, Homo sapiens
  source 1..34: mol_type = tRNA
agccgtgatcgtatagtggttagtactctgcgtt

SEQ ID NO: 871 - RNA, 35 nt, Homo sapiens
  source 1..35: mol_type = tRNA
agccgtgatcgtatagtggttagtactctgcgttg

SEQ ID NO: 872 - RNA, 34 nt, Homo sapiens
  source 1..34: mol_type = tRNA
accgtgatcgtatagtggttagtactctgcgttg

SEQ ID NO: 873 - RNA, 31 nt, Homo sapiens
  source 1..31: mol_type = tRNA
gccgtgatcgtatagtggttagtactctgcg

SEQ ID NO: 874 - RNA, 33 nt, Homo sapiens; identical to SEQ ID NO: 861
  source 1..33: mol_type = tRNA

SEQ ID NO: 875 - RNA, 34 nt, Homo sapiens; identical to SEQ ID NO: 862
  source 1..34: mol_type = tRNA

SEQ ID NO: 876 - RNA, 35 nt, Homo sapiens
  source 1..35: mol_type = tRNA
gccgtgatcgtatagtggttagtactctgcgttgt

SEQ ID NO: 877 - RNA, 31 nt, Homo sapiens
  source 1..31: mol_type = tRNA
ggccgtgatcgtatagtggttagtactctgc

SEQ ID NO: 878 - RNA, 32 nt, Homo sapiens
  source 1..32: mol_type = tRNA
ggccgtgatcgtatagtggttagtactctgcg

SEQ ID NO: 879 - RNA, 34 nt, Homo sapiens; identical to SEQ ID NO: 864
  source 1..34: mol_type = tRNA

SEQ ID NO: 880 - RNA, 35 nt, Homo sapiens; identical to SEQ ID NO: 865
  source 1..35: mol_type = tRNA

SEQ ID NO: 881 - RNA, 36 nt, Homo sapiens
  source 1..36: mol_type = tRNA
ggccgtgatcgtatagtggttagtactctgcgttgt

SEQ ID NO: 882 - RNA, 31 nt, Homo sapiens
  source 1..31: mol_type = tRNA
tgccgtgatcgtatagtggttagtactctgc

SEQ ID NO: 883 - RNA, 32 nt, Homo sapiens
  source 1..32: mol_type = tRNA
tgccgtgatcgtatagtggttagtactctgcg

SEQ ID NO: 884 - RNA, 34 nt, Homo sapiens; identical to SEQ ID NO: 867
  source 1..34: mol_type = tRNA

SEQ ID NO: 885 - RNA, 35 nt, Homo sapiens; identical to SEQ ID NO: 868
  source 1..35: mol_type = tRNA

SEQ ID NO: 886 - RNA, 36 nt, Homo sapiens
  source 1..36: mol_type = tRNA
tgccgtgatcgtatagtggttagtactctgcgttgt

SEQ ID NO: 887 - RNA, 31 nt, Homo sapiens
  source 1..31: mol_type = tRNA
agccgtgatcgtatagtggttagtactctgc

SEQ ID NO: 888 - RNA, 32 nt, Homo sapiens
  source 1..32: mol_type = tRNA
agccgtgatcgtatagtggttagtactctgcg

SEQ ID NO: 889 - RNA, 34 nt, Homo sapiens; identical to SEQ ID NO: 870
  source 1..34: mol_type = tRNA

SEQ ID NO: 890 - RNA, 35 nt, Homo sapiens; identical to SEQ ID NO: 871
  source 1..35: mol_type = tRNA

SEQ ID NO: 891 - RNA, 36 nt, Homo sapiens
  source 1..36: mol_type = tRNA
agccgtgatcgtatagtggttagtactctgcgttgt

(19) United States
(12) Patent Application Publication
(10) Pub. No.: US 2025/0215504 A1
(43) Pub. Date: Jul. 3, 2025

(54) COMPOSITIONS AND METHODS OF USING HisGTG TRANSFER RNAS (tRNAs)

(71) Applicant: Thomas Jefferson University, Philadelphia, PA (US)

(72) Inventor: Isidore Rigoutsos, Astoria, NY (US)

(21) Appl. No.: 18/982,106

(22) Filed: Dec. 16, 2024

Related U.S. Application Data
(63) Continuation of application No. 17/710,204, filed on Mar. 31, 2022, now abandoned.
Application No. 17/710,204 is a continuation of application No. 16/071,231, filed on Jul. 19, 2018, now abandoned.
(60) Provisional application No. 62/292,036, filed on Feb. 5, 2016.

(51) Int. Cl. C12Q 1/6886; C12Q 1/6883

(52) U.S. Cl. CPC C12Q 1/6886; C12Q 1/6883; C12Q 2600/158; C12Q 2600/178

(57) ABSTRACT

The present invention includes a method for analyzing tRNA^{HisGTG} fragments. In one aspect, the present invention includes a method of identifying a subject in need of therapeutic intervention to treat and/or prevent a disease or condition, disease recurrence, or disease progression comprises characterizing the identity of tRNA^{HisGTG} fragments. The invention further includes diagnosing, identifying or monitoring a disease or condition, a panel of engineered oligonucleotides, a kit for a high-throughput assay, and a method and system for identifying tRNA^{HisGTG} fragments.

### CROSS-REFERENCE TO RELATED APPLICATIONS

The present application is a continuation of and claims priority to U.S. patent application Ser. No. 17/710,204, filed Mar. 31, 2022, which is a continuation of and claims priority to U.S. patent application Ser. No. 16/071,231, filed Jul. 19, 2018, abandoned, which is a 35 U.S.C. §371 national phase application from International Application No. PCT/US2017/016560, filed Feb. 3, 2017, which claims priority under 35 U.S.C. §119(e) to U.S. Provisional Patent Application No. 62/292,036, filed Feb. 5, 2016, each of which applications is incorporated herein by reference in its entirety.

### INCORPORATION-BY-REFERENCE OF SEQUENCE LISTING

The present application contains a Sequence Listing which has been submitted in XML and is hereby incorporated by reference in its entirety. Said Sequence Listing, created on Dec. 11, 2024, is named 205961-7013US3 Sequence Listing.xml and is 782,336 bytes in size.

### BACKGROUND OF THE INVENTION

Improvements in deep-sequencing have been facilitating new discoveries that support a framework in which non-coding RNAs (ncRNAs) are as important as proteins. Accumulating data have led to the discovery of new families of ncRNAs and to an improved understanding of established families such as microRNAs (miRNAs) through the discovery of miRNA isoforms.

Transfer RNAs (tRNAs) are ancient molecules that are present in all three life kingdoms. tRNAs are integral components of the process of translation. Many fragments of the precursor and mature tRNAs co-exist with the full length mature tRNAs. In the early days, tRFs were thought to be degradation products or transcriptional noise but follow-up experimental work showed for several of them that they are functionally important.

Early studies with human cell lines established four structural categories of tRFs (FIG. 1): a) 5′-tRNA halves or ‘5-tRHs’ (dashed curves) are ˜34 nucleotides (nt) long and produced from the mature tRNA through cleavage at the anticodon, a step that is catalyzed by the enzyme Angiogenin (ANG); b) 3′-tRNA halves or ‘3′-tRHs’ (dotted black curves) are the tail-half of the mature tRNA following cleavage at the anticodon; c) 5′-tRFs (dotted light gray curves) are typically ˜20 nt long and produced through cleavage of the mature tRNAs at the D-loop; and, finally, d) 3′-tRFs (light gray continues curves) that are also typically ˜20 nt long and produced through cleavage at the T-loop. Recently, a novel category of tRFs that depends strongly on cell type was added to the tRF framework and was named ‘internal tRFs’ or ‘i-tRFs’ (FIG. 1, black continuous curves). i-tRFs begin and end in the interior of the mature tRNA's span. i-tRFs, as well as the number of different existing i-tRFs, are currently uncharacterized.

With regard to function, tRFs affect cell growth, cell proliferation, cellular response to DNA damage, translation initiation, and stress granule formation. tRFs have also been shown to be influenced by diet and trauma and to affect gene production in sperm, to inhibit HIV replication in HIV-infected human MT4 T-cells, or to promote viral replication following RSV infection. tRFs from all five structural categories shown in FIG. 1 were shown to be loaded on Argonaute (Ago), and, thus, they function in the RNAi pathway. For instance, i-tRFs can act as tumor suppressors by competing for binding to RNA binding proteins. It was reported recently that, in human tissues, tRFs are produced by nuclearly-encoded as well as mitochondrially-encoded tRNAs. tRFs were also shown to be produced constitutively, and to have quantized lengths and specific starting/ending points. In fact, the composition and abundance of tRFs were shown to depend on tissue type, tissue state, disease subtype, and a person's gender, population, and race. Considering the large diversity of tRFs and their strong tissue-specificity, very little is known about their roles in different cellular contexts.

Therefore, a need exists for uncovering key tRNA fragments having functional and regulatory roles in diseased and healthy cells. This invention addresses this need.

### BRIEF SUMMARY OF THE INVENTION

The invention provides a method of identifying a subject in need of therapeutic intervention to treat and/or prevent a disease, condition, disease recurrence or disease progression. The invention further provides a method of diagnosing, identifying or monitoring a disease or condition in a subject in need thereof. The invention further provides a method of identifying a cell's tissue of origin to treat and/or prevent a disease or condition, disease recurrence, or disease progression in a subject in need thereof. The invention further provides a set of engineered oligonucleotides. The invention further provides a kit for high-throughput analysis of tRNA^{HisGTG} fragment in a sample.

In certain embodiments, the method comprises isolating at least one tRNA^{HisGTG} fragment from a sample obtained from the subject. In other embodiments, the method comprises characterizing the tRNA^{HisGTG} fragment and its relative abundance in the sample to identify a signature. In yet other embodiments, when the signature is indicative of a diagnosis of the disease, condition, disease recurrence or disease progression, treatment of the subject is recommended.

In certain embodiments, the tRNA^{HisGTG} is at least one selected from the group consisting of a 5′-tRNA fragment (5′-tRF), an internal tRNA fragment (i-tRF), a 3′-tRNA fragment (3′-tRF), a 5′-tRNA half, and a 3′-tRNA half.

In certain embodiments, the tRNA^{HisGTG} fragment is at least one selected from the group consisting of a 5′-tRNA fragment (5′-tRF), an internal-tRNA fragment (i-tRF) and a 3′-tRNA fragment (3′-tRF).

In certain embodiments, the tRNA^{HisGTG} fragment has a length in the range of about 15 nucleotides to about 80 nucleotides.

In certain embodiments, the nucleic acid sequence of the tRNA^{HisGTG} fragment comprises at least one selected from the group consisting of SEQ ID NOs: 1-858.

In certain embodiments, the tRNA^{HisGTG} fragment is post-transcriptionally modified with at least one selected from the group consisting of guanylation, uridylation, adenylation, P, cP, OH, and aa.

In certain embodiments, the post-transcriptionally modified tRNA^{HisGTG} fragment interacts with Argonaute (Ago).

In certain embodiments, the relative abundance of the tRNA^{HisGTG} fragment is measured as a ratio of the tRNA^{HisGTG} fragment and another RNA transcript of interest.

In certain embodiments, the tRNA^{HisGTG} fragment is at least one selected from the group consisting of a 5′-tRNA fragment (5′-tRF), an internal-tRNA fragment (i-tRF) and a 3′-tRNA fragment (3′-tRF), and wherein the relative abundance is high in a hormone dependent cancer,

In certain embodiments, the another RNA transcript of interest is another tRNA^{HisGTG} fragment that differs by a single nucleotide.

In certain embodiments, the sample is isolated from a cell, tissue or body fluid obtained from the subject.

In certain embodiments, the body fluid is at least one selected from the group consisting of amniotic fluid, aqueous humour and vitreous humour, bile, blood serum, breast milk, cerebrospinal fluid, cerumen, chyle, chyme, endolymph and perilymph, exudates, feces, female ejaculate, gastric acid, gastric juice, lymph, mucus, pericardial fluid, peritoneal fluid, pleural fluid, pus, rheum, saliva, sebum, serous fluid, semen, smegma, sputum, synovial fluid, sweat, tears, urine, vaginal secretion, and vomit.

In certain embodiments, the sample is at least one selected from the group consisting of a peripheral blood cell, a tumor cell, a circulating tumor cell, an exosome, a bone marrow cell, a breast cell, a lung cell, a pancreatic cell, a prostate cell, a brain cell, a liver cell, and a skin cell.

In certain embodiments, the method comprises hybridizing the tRNA^{HisGTG} fragment obtained from a cell obtained from the subject to a panel of oligonucleotides engineered to detect the tRNA^{HisGTG} fragment. In other embodiments, the method comprises analyzing levels of the tRNA^{HisGTG} fragment present in the cell. In yet other embodiments, a differential in the measured tRNA^{HisGTG} fragment levels compared to a reference is indicative of a diagnosis or identification of breast cancer in the subject. In yet other embodiments, the method comprises providing a treatment regimen to the subject dependent on the differential in the measured tRNA^{HisGTG} fragment levels to the reference,

In certain embodiments, the disease or condition is a cancer selected from the group consisting of breast cancer, lung cancer, pancreatic cancer, prostate cancer, liver cancer and eye cancer.

In certain embodiments, the disease or condition is a neurological disease selected from the group consisting of Alzheimer's disease, Parkinson's disease and amyotrophic lateral sclerosis.

In certain embodiments, the set of engineered oligonucleotides comprises a mixture of oligonucleotides that are about 15 to about 50 nucleotides in length and capable of hybridizing at least one tRNA^{HisGTG} fragment.

In certain embodiments, the nucleic acid sequence of the at least one tRNA^{HisGTG} fragment comprises at least one selected from the group consisting of SEQ ID NOs: 1-858.

In certain embodiments, the kit for high-throughput analysis of tRNA^{HisGTG} fragment in a sample comprises the set of engineered oligonucleotides of the invention; hybridization reagents; and tRNA fragment isolation reagents.

In certain embodiments, the method comprises isolating at least one tRNA^{HisGTG} fragment from a cell obtained from the subject. In other embodiments, the method comprises characterizing the identity of the tRNA^{HisGTG} fragment and its relative abundance in the cell to identify a signature. In yet other embodiments, the signature is indicative of the cell's tissue of origin. In yet other embodiments, the method comprises providing a treatment regimen to the subject dependent on the cell's tissue of origin.

In certain embodiments, the nucleic acid sequence of the at least one tRNA^{HisGTG} fragment comprises at least one selected from the group consisting of SEQ ID NOs: 1-858.

In certain embodiments, the subject is a mammal. In other embodiments, the subject is a human.

### BRIEF DESCRIPTION OF THE DRAWINGS

The following detailed description of certain embodiments of the invention will be better understood when read in conjunction with the appended drawings. For the purpose of illustrating the invention, there are examples shown in the drawings illustrative embodiments.

It should be understood, however, that the invention is not limited to the precise arrangements and instrumentalities of the embodiments shown in the drawings.

FIG. 1 is an illustration showing the typical tRNA cloverleaf secondary structure with the four previously known structural categories of tRFs and the novel structural category (i-tRFs) superimposed. In practice, a typical tRNA may produce one or more distinct fragments. The sequence of the tRNA (SEQ ID NO: 859) is shown.

FIG. 2 is an alignment showing 41 abundant fragments from the 5′-region of the tRNA^{HisGTG} locus that are present in breast cancer tissue and cell lines. RNA111.HisGTG (SEQ ID NO: 860), from the reverse strand of chr 1 between locations 147774845 and 147774916 (hg19), was used to align the fragments (Chan & Lowe, 2009, Nucleic acids research 37, D93-97). SHOT-RNAs are noted (SEQ ID NOs: 861-863 lack the −1 position, SEQ ID NOs: 864-866 contain G at the −1 position, SEQ ID NOs: 867-869 contain U at the −1 position, and SEQ ID NOs: 870-872 contain A at the −1 position). Sequences of certain 5′-halves are shown (SEQ ID NOs: 85 and 873-876 lack the −1 position, SEQ ID NOs: 877-881 contain G at the −1 position, SEQ ID NOs: 882-886 contain U at the −1 position, and SEQ ID NOs: 887-891 contain A at the −1 position). Sequences of certain 5′-tRFs are shown (SEQ ID NOs: 71-84 lack the −1 position, SEQ ID NOs: 53-66 contain U at the −1 position, and SEQ ID NOs: 2-15 contain A at the −1 position. A 5′-halves proxy sequence is shown (SEQ ID NO: 85). Sequences of certain i-tRFs are shown (from top to bottom, SEQ ID NOs: 399, 661, 768, 569, 682, 662, 585, 769, 753, 381, 663, 586, 770, 507, 664, 587, 771, and 508). The anticodon and its loop as well as the D-loop are highlighted in grey. The ‘>’ and ‘<’ arrows show paired-up bases in the secondary structure.

FIGS. 3A-3B are a series of graphs showing that the internal tRFs (i-tRFs) are a rich, tissue-dependent novel category. Shown are the i-tRFs' starting positions, spans, and lengths for lymphoblastoid cells (FIG. 3A) and breast cancer samples from The Cancer Genome Atlas repository (FIG. 3B). Position numbers refer to the +1 position of the mature tRNA. Gray boxes highlight the D- and T-loops, and the anticodon. Bar shading captures the respective fragment's abundance. Right wall projections show proportionally how many distinct i-tRFs are produced from each tRNA region.

FIG. 4 is a set of graphs showing the tissue-state-dependence of the lengths of i-tRFs and 5′-tRFs.

FIG. 5 is a set of graphs showing that tRF profiles depend on an person's race both in health and disease. Top panel shows a separation of normal breast samples in White and Black individuals. FIG. 5, bottom panel, shows a separation of samples in White and Black individuals with triple negative breast cancer. All samples are from The Cancer Genome Atlas collection.

FIGS. 6A-6P are a set of graphs showing the abundance ratios of −1T 5′-tRFs from tRNA^{HisGTG} that end at consecutive positions within the mature tRNA for several TCGA cancers. Values are plotted only for statistically significant tRFs. Y-axis: log10. These plots correspond to the log10 of the mean ratio of (abundance of His(−1) 5′-tRF ending at position i)/(abundance of His(−1) 5′-tRF ending at position i+1), for all 32 cancer types. The various panels of this figure use the abbreviations shown in FIG. 15. In each sample, the tRF abundances were normalized by converting them to reads-per-million (RPM) values. E.g. two such consecutive fragments are T-GCCGTGATCGTATAGT (SEQ ID NO: 54) and T-GCCGTGATCGTATAGT-G (SEQ ID NO: 55). The ratios shown are for normal (grey) and cancer (black) samples across 32 TCGA cancers.

FIG. 7 is a set of graphs showing Ago-loaded His(−1) tRFs in three BRCA cell lines. Top panel: 5′-uridylated fragments (contain T at position −1). Bottom panel: 5′-guanylated fragments (contain G at position −1). Note the dependence on the cell line and the identity of the 5′ addition to position −1. The X-axis is the tRF's position in tRNA^{HisGTG}. The D-loop, anticodon loop, and anticodon are also shown highlighted.

FIG. 8 is a graph showing validation of an i-tRF AspGTC|15.35.21 in BRCA clinical samples using dumbbell-PCR. Subjects 3, 6, 7, 8, 10 and 11 are ER+.

FIG. 9 is an image showing a Pearson correlation of HisGTG −1T 5′-tRFs (grey) and i-tRFs (black) for 1,049 TCGA BRCA samples. Shown correlations are significant (P-val<0.01). tRFs listed by the location of their endpoints. Cells with asterisks (“*”) correspond to anti-correlated pairs.

FIG. 10 is a graph depicting a principal component analysis (PCA) of the experiments presented herein in which cells were transfected with a −1T TRF from tRNA^{HisGTG} or a control.

FIG. 11 is a graph depicting a principal component analysis (PCA) where transfections of two cell lines (BT-20 and MDA-MB-468) with two different tRFs from tRNA^{HisGTG} are compared. Note the more pronounced difference in response to the transfections in the MDA-MB-468 cell line.

FIG. 12 is a table listing 66 tRFs of interest that begin at position −1 of isodecoders of tRNA^{HisGTG} (SEQ ID NOs: 1-66). These tRFs were selected from 20,722 distinct tRFs generated by the analysis of the 10,274 datasets mentioned elsewhere herein.

FIG. 13 is a table listing 21 tRFs of interest that begin at position +1 of isodecoders of tRNA^{HisGTG} (SEQ ID NOs: 67-87). These tRFs were selected from 20,722 distinct tRFs generated by the analysis of the 10,274 datasets mentioned elsewhere herein.

FIGS. 14A-14K are a set of tables listing 771 tRFs that begin at positions other than −1 or +1 of isodecoders of tRNA^{HisGTG} (SEQ ID NOs: 88-858). These tRFs were selected from 20,722 distinct tRFs generated by the analysis of the 10,274 datasets mentioned elsewhere herein.

FIG. 15 is a table listing the abbreviations for the type of cancer referred to herein.

FIGS. 16A-16B are a set of table listing protein localization of mRNAs that are correlated with tRFs from tRNA^{HisGTG}, by cancer.

### DETAILED DESCRIPTION OF THE INVENTION

Definitions Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “complementary sequence” or “complement” is meant a nucleic acid base sequence that can form a double-stranded structure by matching base pairs to another polynucleotide sequence. Base pairing occurs through the formation of hydrogen bonds, which may be Watson-Crick, Hoogsteen or reversed Hoogsteen hydrogen bonding, between complementary nucleobases. For example, adenine and thymine are complementary nucleobases that pair through the formation of hydrogen bonds.

In this disclosure, “comprises,” “comprising,” “containing” and “having” and the like can have the meaning ascribed to them in U.S. Patent law and can mean “includes,” “including” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, eye cancer, lymphoma, leukemia, lung cancer and the like.

“Detect” refers to identifying the presence, absence or amount of the biomarker to be detected.

The phrase “differentially present” refers to differences in the quantity and/or the frequency of a biomarker present in a sample taken from subjects having a disease as compared to a control subject. A biomarker can be differentially present in terms of quantity, frequency or both. A polypeptide or polynucleotide is differentially present between two samples if the amount or frequency of the polypeptide or polynucleotide in one sample is statistically significantly different (either higher or lower) from the amount of the polypeptide or polynucleotide in the other sample, such as reference or control samples. Alternatively or additionally, a polypeptide or polynucleotide is differentially present between two sets of samples if the amount or frequency of the polypeptide or polynucleotide in samples of the first set, such as diseased subjects' samples, is statistically significantly (either higher or lower) from the amount of the polypeptide or polynucleotide in samples of the second set, such reference or control samples. A biomarker that is present in one sample, but undetectable in another sample is differentially present.

A “disease” is a state of health of an animal wherein the animal cannot maintain homeostasis, and wherein if the disease is not ameliorated then the animal's health continues to deteriorate. A “disease subtype” is a state of health of an animal wherein animals with the disease manifest different clinical features or symptoms. For example, Alzheimer's disease includes at least three subtypes, inflammatory, non-inflammatory, and cortical.

A “disorder” as used herein, is used interchangeably with “condition,” and refers to a state of health in an animal, wherein the animal is able to maintain homeostasis, but in which the animal's state of health is less favorable than it would be in the absence of the disorder. Left untreated, a disorder does not necessarily cause a further decrease in the animal's state of health.

By “effective amount” is meant the amount required to reduce or improve at least one symptom of a disease relative to an untreated patient. The effective amount of active compound(s) used to practice the present invention for therapeutic treatment of a disease varies depending upon the manner of administration, the age, body weight, and general health of the subject.

As used herein “endogenous” refers to any material from or produced inside an organism, cell, tissue or system.

The term “expression” as used herein is defined as the transcription and/or translation of a particular nucleotide sequence driven by its promoter.

By “fragment” is meant a portion of a polynucleotide or nucleic acid molecule. This portion contains, preferably, at least 10%, 20%, 30%, 40%, 50%, 60%, 70%, 80%, 90%, 91%, 92%, 93%, 94%, 95%, 96%, 97%, 98%, or 99% of the entire length of the reference nucleic acids. A fragment may contain 10, 20, 30, 40, 50, 60, 70, 80, 90, or 100, 200, 300, 400, 500, 600, 700, 800, 900, 1000, 1500, 2000 or 2500 (and any integer value in between) nucleotides. The fragment, as applied to a nucleic acid molecule, refers to a subsequence of a larger nucleic acid. The fragment can be an autonomous and functional molecule. A fragment may contain modifications at neither, one or both of its termini. A modification can include but is not limited to a phosphate, a cyclic phosphate, a hydroxyl, and an amino acid. A “fragment” of a nucleic acid molecule may be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Similar” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are similar at that position. The percent of similarity between two sequences is a function of the number of matching or similar positions shared by the two sequences divided by the number of positions compared×100. For example, if 6 of 10 of the positions in two sequences are matched or similar then the two sequences are 60% similar. By way of example, the DNA sequences ATTGCC and TATGGC share 50% similarity. Generally, a comparison is made when two sequences are aligned in a way that maximizes their similarity.

As used herein, the term “inhibit” is meant to refer to a decrease in biological state. For example, the term “inhibit” may be construed to refer to the ability to negatively regulate the expression, stability or activity of a protein, including but not limited to transcription of a protein mRNA, stability of a protein mRNA, translation of a protein mRNA, stability of a protein polypeptide, a protein post-translational modifications, a protein activity, a protein signaling pathway or any combination thereof.

Further, the term “inhibit” may be construed to refer to the ability to negatively affect the expression, stability or activity of a miRNA or tRNA or tRNA fragment, wherein such inhibition of the miRNA or tRNA or tRNA fragment may result in the modulation of a gene including but not limited to a protein's mRNA abundance, the stability of a protein's mRNA, the translation of a protein's mRNA, the stability of a protein, the post-translational modifications of a protein, and/or the activity of a protein.

“Instructional material,” as that term is used herein, includes a publication, a recording, a diagram, or any other medium of expression that may be used to communicate the usefulness of the compounds and/or methods of the invention. In some instances, the instructional material may be part of a kit useful for diagnosing and/or effecting alleviating or treating the various diseases or conditions recited herein. Optionally, or alternately, the instructional material may describe one or more methods of diagnosing and/or alleviating the diseases or conditions in a cell or a tissue of a mammal. The instructional material of the kit may, for example, be affixed to a container that contains the compounds of the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “mitochondrial tRNAs” is used to refer to tRNAs encoded in the mitochondrial genome. The term “nuclear tRNAs” is used to refer to tRNAs encoded in the nuclear genome. In certain non-limiting embodiments, the distinction of the origin of the DNA precursor template may not be entirely accurate from a biological standpoint: as reported in Telonis et al., 2014, Front Genet, 5:344; Telonis et al., 2015, RNA Biol, 12:4, 375-380), the nuclear genome contains numerous full-length lookalikes of mitochondrial tRNAs. It is currently unclear whether these nuclear lookalike sequences are transcribed or whether they act as tRNAs; thus, special consideration is needed to discard sequencing reads that may map to those lookalikes and to the tRNA space, which are defined elsewhere herein.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By “isolated polynucleotide” is meant a nucleic acid (e.g., a DNA or an RNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a rRNA, cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

The term “oligonucleotide panel” or “panel of oligonucleotides” refers to a collection of one or more oligonucleotides that may be used to identify DNA (e.g. genomic segments comprising a specific sequence, DNA sequences bound by particular protein, etc.) or RNA (e.g. mRNAs, microRNAs, tRNAs, rRNAs etc.) through hybridization of complementary regions between the oligonucleotides and the DNA or RNA. If the sought molecule is RNA, it is commonly converted to DNA through a reverse transcription step). The oligonucleotides may include complementary sequences to known DNA or known RNA sequences. The oligonucleotides may be engineered to be between about 5 nucleotides to about 40 nucleotides, or about 5 nucleotides to about 30 nucleotides, or about 5 nucleotides to about 20 nucleotides, or about 5 nucleotides to about 15 nucleotides in length. The term “oligonucleotide panel” or “panel of oligonucleotides” could also refer to a system and accompanying collection of reagents that, in addition to being able to hybridize to molecules containing a complementary sequence, can also ensure that the identified molecule's 3′ terminus matches precisely the 3′ terminus of the sought molecule, or that the identified molecule's 5′ terminus matches precisely the 5′ terminus of the sought molecule, or both: this ability is unlike what can be achieved by conventional assays such as e.g. Affymetrix chips, and methods (e.g. “dumbbell-PCR”) and systems (e.g. the Fireplex system of Firefly BioWorks) that can achieve this are now beginning to be available.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormally high level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art. The term “underexpressed” tumor antigen or “underexpression” of the tumor antigen is similarly analogous.

The term “overexpressed” tumor promoter or “overexpression” of the tumor promoter is intended to indicate an abnormally high level of expression of the tumor promoter RNA or protein in a cell from a disease area like a solid tumor within a specific tissue or organ of the patient relative to the level of expression in a normal cell from that tissue or organ. Patients having solid tumors or a hematological malignancy characterized by overexpression of the tumor promoter can be determined by standard assays known in the art. The term “underexpressed” tumor promoter or “underexpression” of the tumor promoter is similarly analogous.

The term “overexpressed” tumor suppressor or “overexpression” of the tumor suppressor is intended to indicate an abnormally high level of expression of the tumor suppressor RNA or protein in a cell from a specific area within a specific tissue or organ of an individual relative to the level of expression under typical circumstances in a cell from that tissue or organ. Individuals having characteristic overexpression of the tumor suppressor can be determined by standard assays known in the art. The term “underexpressed” tumor suppressor or “underexpression” of the tumor suppressor is similarly analogous.

The terms “patient,” “subject,” “individual,” and the like are used interchangeably herein, and refer to a human or non-human mammal, or cells thereof whether in vitro or in situ, amenable to the methods described herein. Non-human mammals include, for example, livestock and pets, such as ovine, bovine, porcine, canine, feline and murine mammals. The term “subject” is intended to include living organisms in which an immune response can be elicited (e.g., mammals). Examples of subjects include humans, dogs, cats, mice, rats, and transgenic species thereof. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which may be hydrolyzed into the monomeric “nucleotides.” The monomeric nucleotides may be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means. The following abbreviations for the commonly occurring nucleic acid bases are used. “A” refers to adenosine, “C” refers to cytosine, “G” refers to guanosine, “T” refers to thymidine, and “U” refers to uridine. The term “RNA” as used herein is defined as ribonucleic acid. The term “recombinant DNA” as used herein is defined as DNA produced by joining pieces of DNA from different sources.

As used herein, the term “population” refers to individuals of either sex that belong to the same race and originate from the same geographical area.

When referring to the phosphatase status of a fragment's 5′- and 3′-termini, the notation “X/Y” is used herein where X, Y can be: hydroxyl (OH), phosphate (P), cyclic phosphate (cP), or amino acid (aa). E.g., “P/cP” refers to fragments with a P at the 5′- and a cP at the 3′-terminus. tRFs of the “P/OH” type are referred to as “canonical.” All other tRF types are “non-canonical.”

As used herein, the terms “prevent,” “preventing,” “prevention,” and the like refer to reducing the probability of developing a disease or condition in a subject, who does not have, but is at risk of or susceptible to developing a disease or condition.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The terms “purified” or “biologically pure” refer to material that is free to varying degrees from components which normally accompany it as found in its native state. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “Race” refers to a taxonomic rank below the species level, a collection of genetically differentiated human populations defined by phenotype. White (Wh) is the National Health Institute/The Cancer Genome Atlas (NIH/TCGA) designation for a person with origins in any of the original peoples of the far Europe, the Middle East, or North Africa. Black or African American (B/Aa) is the NIH/TCGA designation for a person with origins in any of the black racial groups of Africa.

A “recyclable tRNA” refers to a tRNA that is aminoacylated and can be repeatedly reaminoacylated with an amino acid (e.g., an unnatural amino acid) for the incorporation of the amino acid (e.g., the unnatural amino acid) into one or more polypeptide chains during translation.

By “reduces” or “decreases” is meant a negative alteration of at least 10%, 25%, 50%, 75%, or 100%.

By “reference” is meant a standard or control. A “reference” is also a defined standard or control used as a basis for comparison.

As used herein, “relative abundance” refers to the ratio of the quantities of two or more molecules of interest (e.g. rRNAs, rRNA fragments, miRNAs, etc.) present in a sample. The relative abundance of two or more molecules of interest in a given sample may differ from the relative abundance of the same two or more molecules in a second sample. The terms “tRNA fragment” or “tRF” are all used to refer to short non-coding RNAs generated from a tRNA locus. tRNA fragments have lengths that range from 10 to 50 or more nucleotides. The tRF notation as introduced in Telonis et al., 2015, Oncotarget 6:28, 24797-24822, e.g. trna111_HisGTG_1_-_147774845_147774916@1.23.23 denotes a fragment from the isodecoder of the mature tRNA^{HisGTG} that is located on chromosome 1, on the reverse strand, between locations 147774845 and 147774916 inclusive, and begins at position 1 of the mature tRNA, ends at position 23 of the mature tRNA, and is 23 nucleotides (nt) long. The terms “tRNA HisGTG” and “HisGTG tRNA” and “tRNA^{HisGTG}” are used interchangeably herein.

As used herein, the tRNA fragments from His that begin at position “−1” are referred to as 5′-tRFs.

As used herein, “sample” or “biological sample” refers to anything, which may contain the biomarker (e.g., polypeptide, polynucleotide, or fragment thereof) for which a biomarker assay is desired. The sample may be a biological sample, such as a biological fluid or a biological tissue. In certain embodiments, a biological sample is a tissue sample including pulmonary vascular cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the percentage of biomarker-detected subjects with a particular disease.

As used herein, “sample” or “biological sample” refers to anything, which may contain the biomarker (e.g., polypeptide, polynucleotide, or fragment thereof) for which a biomarker assay is desired. The sample may be a biological sample, such as a biological fluid or a biological tissue. In certain embodiments, a biological sample is a tissue sample including pulmonary vascular cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the percentage of biomarker-detected subjects with a particular disease.

The terms “short RNA profile” or “RNA profile” or “tRNA profile” or “tRNA fragment profile” are used interchangeably and refer to a genetic makeup of the RNA molecules that are present in a sample, such as a cell, tissue, or subject. Optionally, the abundance of an RNA molecule that is part of an RNA profile may also be sought. Optionally, other attributes of an RNA molecule that is part of an RNA profile may also be sought and include but are not limited to a molecule's location within the genomic locus of origin, the molecule's starting point, the molecule's ending point, the molecule's length, the identity of the molecule's terminal modifications, etc. The RNA molecules that can be used to form such a profile can be miRNAs, mRNAs, rRNAs, tRNAs fragments, etc. as well as combinations thereof.

The term “signature” or “RNA signature” as used herein refers to a subset of an RNA profile and comprises the identity of one or more molecules that are selected from an RNA profile and optionally one or more of the attributes of the one or more molecules that are selected from the RNA profile.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disease or condition being treated. The therapeutically effective amount will vary depending on the compound, the disease and its severity and the age, weight, etc., of the subject to be treated.

A “suppressor RNA” refers to a tRNA that alters the reading of a messenger RNA (mRNA) in a given translation system, e.g., by providing a mechanism for incorporating an amino acid into a polypeptide chain in response to a selector codon. For example, a suppressor tRNA can read through, e.g., a stop codon, a four base codon, a rare codon, and/or the like.

The term “diagnostic” refers to a method yielding a diagnosis to help identifying the nature or cause of a disease, disorder, illness, condition or problem. In some instances, a diagnosis is performed for a subject by systematic analysis of the background or history, examination of the signs or symptoms of the condition, evaluation of the research or test results and investigation of the causes of the condition.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disease or condition being treated. The therapeutically effective amount will vary depending on the compound, the disease and its severity and the age, weight, etc., of the subject to be treated.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or improving a disease or condition and/or symptom associated therewith. It will be appreciated that, although not precluded, treating a disease or condition does not require that the disease, condition or symptoms associated therewith be completely ameliorated or eliminated.

The terms “tRNA^{HisGTG},” “tRNAHisGTG,” “HisGTG tRNA,” “tRNA fragment,” or “tRF” are functional short non-coding RNAs generated from a tRNA locus. HisGTG tRNAs have lengths that range from 10 to 80 or more nucleotides, Categories of tRNA^{HisGTG} fragments include the 5′-tRFs, the i-tRFs, the 3′-tRFs, the 5′-halves, and the 3′-halves. The term “tRNA locus” refers to the genomic region that includes a tRNA gene and gives rise to the tRNA transcript. A given tRNA locus can produce zero, one, or more molecules belonging to zero, one, or more of the four structural categories.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Description

The present invention includes methods and compositions of analyzing tRNA^{HisGTG} fragments. tRNAs are ancient non-coding RNAs (ncRNAs) that have been heretofore understood to be molecules with well-defined roles confined to the translation of messenger RNA (mRNA) into amino acid sequences. As such, tRNAs are present in archaea, bacteria, and eukaryotes. The conventional understanding had been that a genomic tRNA locus produces a single transcript that is processed to give rise to the mature tRNA. Described herein, tRNA loci also produce fragments that are important novel regulators with roles in cellular physiology, post-transcriptional regulation, and so forth. The specifics of how tRNA fragments effect these roles are currently understood poorly. The present invention utilizes tRNA^{HisGTG} fragment profiling to identify subjects in need of therapeutic intervention.

In one aspect, the invention provides a method of identifying a subject in need of therapeutic intervention to treat a disease or disease progression. In certain embodiments, the method comprises isolating at least one tRNA^{HisGTG} fragment from a sample obtained from the subject; characterizing the tRNA^{HisGTG} fragment and its relative abundance with regard to another transcript in the sample to identify a signature, wherein when the signature is indicative of a diagnosis of the disease treatment of the subject is recommended. In certain embodiments, the subject is a human.

In another aspect, the invention provides a method of identifying a cell's tissue of origin to treat a disease or disease progression or disease recurrence in a subject in need thereof. In certain embodiments, the method comprises isolating fragments of tRNAs from a cell obtained from the subject; characterizing the fragments of tRNA and their relative abundance in the cell to identify a signature, wherein the signature is indicative of the cell's tissue of origin, or the disease status of the tissue of origin; and providing a treatment regimen to the subject dependent on the cell's tissue of origin, or the disease status of the tissue of origin.

HisGTG tRNA Fragments

Analysis of tRNA^{HisGTG} fragment profiles or signatures in one or more cells can lead to the discovery of tRNA fragment signatures present in healthy cells or diseased cells. tRNA^{HisGTG} fragment signatures in one or more cells, or a tissue may be used to identify a diseased cell, disease progression, or disease recurrence in a subject. Thus, the subject can be identified as in need of therapeutic intervention to delay the onset of, reduce, improve, and/or treat a disease or condition, such as breast cancer, in a subject in need thereof. In some embodiments, the disease or condition is a cancer, an immune or autoimmune disease or a neurological or neurodegenerative disease. In some embodiments, the disease or condition is a cancer selected from the group consisting of breast cancer, lung cancer, pancreatic cancer, prostate cancer, liver cancer and eye cancer. In other embodiments, the disease or condition is a neurological disease selected from the group consisting of Alzheimer's disease, Parkinson's disease and amyotrophic lateral sclerosis.

Also provided is a panel of engineered oligonucleotides comprising a mixture of oligonucleotides that are about 15 to about 50 nucleotides (nts) in length and capable of hybridizing tRNA^{HisGTG} fragments and/or tRNAs, wherein the tRNA^{HisGTG} fragments are generally at least 15 nts in length and the tRNA^{HisGTG} fragments are generally less than 80 nts in length. The panel may include one or more oligonucleotides that may be used to identify one or more tRNA^{HisGTG} fragments through hybridization of complementary regions between the oligonucleotides and the tRNA^{HisGTG}, or related techniques that are well known to those skilled in the art. The oligonucleotides may include complementary sequences to known tRNA sequences, such as tRNA^{HisGTG} fragments. The oligonucleotides may be engineered to be between about 5 nucleotides to about 60 nucleotides, or about 5 nucleotides to about 50 nucleotides, or about 5 nucleotides to about 40 nucleotides, or about 5 nucleotides to about 30 nucleotides, or about 5 nucleotides to about 20 nucleotides, or about 5 nucleotides to about 15 nucleotides in length. In some embodiments, the oligonucleotides can be engineered to be between about 15 nucleotides to about 60 nucleotides, or about 15 nucleotides to about 50 nucleotides in length. The panel may include engineered oligonucleotides that are specific to a cell type, disease type, disease subtype, stage of disease, a patient's sex, a patient's population of origin, a patient's race or other aspect that may differentiate tRNA^{HisGTG} fragment signatures. The kits and oligonucleotide panel may also be used to identify agents that modulate disease, or progression of disease, or disease recurrence, in patient samples, and/or in in vitro or in vivo animal models for the disease at hand.

In another aspect, the invention includes a method for identifying tRNA^{HisGTG} fragments from sequenced reads, typically obtained through next generation sequencing approaches. The method comprises the steps of defining tRNA loci; mapping the sequenced reads to at least one tRNA genomic locus comprising disregarding map locations that differ from the tRNA^{HisGTG} fragments by at least an insertion, deletion, or replacement of a nucleotide, optionally excluding tRNA^{HisGTG} fragments that can also be found at locations outside of the tRNA loci, and disregarding sequenced reads with tRNA intron sequences; mapping sequenced reads that are post-transcriptionally modified; and characterizing the remaining sequenced reads.

Known tRNA loci include the mitochondrial genome loci of mitochondrial tRNA sequences, the nuclear genome loci of nuclear tRNA sequences, and the nuclear genome loci of some mitochondrial tRNA sequences. Currently, there are the 22 known human mitochondrial tRNA sequences in the mitochondrial genome. There are 610 (508 true tRNAs and 102 pseudo-tRNAs) nuclear tRNA sequences in the nuclear genome, as per the public genomic tRNA database “gtRNAdb.” Selenocysteine tRNAs, tRNAs with undetermined anticodon identity, and tRNAs mapping to contigs that were not part of the human chromosome assembly are excluded from the collection of tRNA sequences considered here. There are also eight intervals in the nuclear genome, chr1:+:566062-566129, chr1:+:568843-568912, chr1:−:564879-564950, chr1:−:566137-566205, chr14:+:32954252-32954320, chr1:−:566207-566279, chr1:−:567997-568065, and, chr5:−:93905172-93905240—all given locations are for the hg19/GRCh37 human genome assembly—that correspond to identical instances of seven mitochondrial tRNAs TrpTCA, LysTTT, GlnTTG, AlaTGC (×2), AsnGTT, SerTGA, and, GluTTC, respectively.

The sequenced reads are further mapped to at least one tRNA genomic locus. Sequenced reads that differ from the map location by at least an insertion, deletion, or replacement of a nucleotide are disregarded. For example, two distinct 5′-tRF molecules that would otherwise be indistinguishable can then be differentiated from one another and properly mapped. Also, the misidentification of the genomic origin of a sequenced read that would lead to erroneous results can be avoided.

The human genome is also riddled with many nuclear and mitochondrial tRNA-look-alikes, as well as partial tRNA sequences. Optionally excluding sequenced reads that map to locations both inside and outside of the tRNA loci permits the optional exclusion of the tRNA-like fragments from further consideration.

Also disregarding sequenced reads with tRNA intron sequences improves identification of bona fide tRNA^{HisGTG} fragments. Many tRNAs include intronic sequences.

Sequenced reads that include only exonic sequences of an intron-containing tRNA are included. Sequenced reads that straddle a tRNA's exon-exon junction are further examined for possible mapping outside tRNA loci: any such reads that map outside tRNA loci can be optionally discarded.

tRNA^{HisGTG} molecules are also subject to post-transcriptional modifications. Mature tRNAs are commonly modified with a CCA trinucleotide added to their 3′ end. In certain embodiments, the tRNA^{HisGTG} is post-transcriptionally modified with at least one selected from the group consisting of guanylation, uridylation, adenylation, P, cP, OH, aa. In other embodiments, the post-transcriptionally modified tRNA^{HisGTG} or tRNA^{HisGTG} fragment interacts with Argonaute (Ago).

Without explicit provisions to include these tRNA^{HisGTG} molecules, they and their fragments could be inadvertently excluded from consideration by lacking an exact genomic map location. However, simply allowing an adequate number of mismatches (e.g. replacements) during mapping the nontemplated CCA is not adequate. Prior to mapping, a modification to the genome is created where the trinucleotide CCA is used to replace the three genomic nucleotides immediately downstream of each of the reference mature tRNAs. Special care must be taken. Otherwise, a careless replacement of the genomic sequence downstream from a tRNA by the CCA trinucleotide could inadvertently “erase” part of an adjacent tRNA's sequence as is the case, for example, for some tRNAs in the mitochondrial genome.

The tRNA^{HisGTG} fragments thusly identified are characterized. In certain embodiments, the tRNA^{HisGTG} fragment is selected from the group consisting of a 5′-tRNA half, a 3′-tRNA half, a 5′-tRNA fragment, an internal tRNA fragment, and a 3′-tRNA fragment.

The tRNA^{HisGTG} fragments can be assessed for one or more of, sequence of the tRNA^{HisGTG} fragments, the overall abundance of the tRNA^{HisGTG} fragments based on the number of sequenced reads that mapped to tRNA loci, the relative abundance of a tRNA^{HisGTG} fragments to a reference, the length of a tRNA^{HisGTG} fragment, the starting and ending points of a tRNA^{HisGTG} fragment, the genomic origin of a tRNA^{HisGTG} fragment, the terminal modifications of a tRNA^{HisGTG}, and other analyses known in the art. In certain embodiments, the tRNA^{HisGTG} fragment has a length in the range of about 15 nucleotides to about 80 nucleotides. In certain embodiments, the nucleic acid sequence of the tRNA^{HisGTG} fragment comprises SEQ ID NOs: 1-858. In other embodiments, the relative abundance is measured as a ratio of the tRNA^{HisGTG} and another tRNA that differs by a single nucleotide.

In another aspect, a system is described herein to perform the method of identifying tRNA^{HisGTG} fragments. In certain embodiments, the system comprises a processor that aligns sequenced reads with a genome and processes the alignment. The processor of the system processes the alignments and disregards data from the alignments when the mapped sequenced reads differ from the genome by at least an insertion, deletion, or replacement of a nucleotide; the mapped sequenced reads align to locations in the genome that reside outside of designated tRNA loci; the sequenced reads map to locations in the genome that reside both inside and outside of designated tRNA loci; or the mapped sequenced reads span intron sequences of tRNAs. The portion of the algorithm that is run by the processor of the system and processes the alignments may also have provisions to include sequenced reads that also map outside of tRNA loci, or that correspond to post-transcriptionally modified molecules and would otherwise not align perfectly with the genome.

### Diagnostics

Samples from subjects suffering from a disease or a condition have a specific tRNA^{HisGTG} fragment profile in the cell or cells that are diseased, including metastastic cancer cells. Identifying the cellular origin or tissue origin of a cancer metastasis, or a propensity for a cell to metasize by identifying a tRNA^{HisGTG} fragment profile associated with the cellular origin or tissue origin or a propensity to metasize in a sample obtained from the subject allows the subject to undergo a recommended treatment. In one aspect, the invention includes a method of identifying a cell's tissue of origin to treat a disease or disease progression, or disease recurrence in a subject in need thereof comprising isolating one or more tRNA^{HisGTG} fragment from a cell obtained from the subject; characterizing the tRNA^{HisGTG} fragment, which can include assessing one or more of, overall abundance, relative abundance, length of the fragment, starting and ending points of the fragment, terminal modifications, and so forth, in the cell to identify a signature, wherein the signature is indicative of the cell's tissue of origin, and/or disease status of the tissue of origin; and providing a treatment regimen to the subject dependent on the cell's tissue of origin and/or disease status of the tissue of origin.

In other embodiments, characterizing the tRNA^{HisGTG} fragment that is present in the RNA profile can identify subjects in need of treatment.

In yet other embodiments, the relative abundance of the tRNA^{HisGTG} fragments that are present in the RNA profile can identify subjects in need of treatment. In another approach, diagnostic methods are used to assess tRNA^{HisGTG} fragment profiles in a biological sample relative to a reference (e.g., tRNA^{HisGTG} fragment profile in a healthy cell or tissue or body fluid in a corresponding control sample). Examples of a body fluid may include, but are not limited to, amniotic fluid, aqueous humour and vitreous humour, bile, blood serum, breast milk, cerebrospinal fluid, cerumen, chyle, chyme, endolymph and perilymph, exudates, feces, female ejaculate, gastric acid, gastric juice, lymph, mucus, pericardial fluid, peritoneal fluid, pleural fluid, pus, rheum, saliva, sebum, serous fluid, semen, smegma, sputum, synovial fluid, sweat, tears, urine, vaginal secretion, and vomit.

In certain embodiments, the sample, such as a cell or tissue or body fluid is obtained from the subject. In other embodiments, the cell or tissue or body fluid is isolated from the sample. In other embodiments, the cell or tissue is isolated from a body fluid. The sample may be a peripheral blood cell, a tumor cell, a circulating tumor cell, an exosome, a bone marrow cell, a breast cell, a lung cell, a pancreatic cell, or other cell of the body.

In general, characterizing the tRNA^{HisGTG} fragments identifies a signature that may be indicative of a diagnosis of a disease or condition. The character of the tRNA^{HisGTG} fragments in the sample may be compared with a reference, such as other tRNA fragments present within the cell, a healthy cell or a diseased cell will yield a relative abundance of the tRNA^{HisGTG} fragments to identify a signature. The signature may be established by comparing the tRNA^{HisGTG} fragment locations within the genomic loci of origin, the starting and ending points of the tRNA fragments, the length of the tRNA fragments, and any other feature of the fragments as compared to other tRNA fragments within the same sample or another sample or reference to distinguish a diseased state, a propensity to develop a disease or condition, and/or the absence of a disease or condition. In certain embodiments, the relative abundance is measured as a ratio of the tRNA^{HisGTG} fragment and another tRNA fragment that differs by a single nucleotide. The skilled artisan will appreciate that the diagnostic can be adjusted to increase sensitivity or specificity of the assay. In general, any significant increase (e.g., at least about 10%, 15%, 30%, 50%, 60%, 75%, 80%, or 90%) in the level of a polynucleotide or polypeptide biomarker in the subject sample relative to a reference may be used to diagnose a diseased state, a propensity to develop a disease or condition, and/or the absence of a disease or condition.

Accordingly, a tRNA^{HisGTG} fragment profile may be obtained from a sample from a subject and compared to a reference tRNA^{HisGTG} fragment profile obtained from a reference cell or tissue or body fluid, so that it is possible to classify the subject as belonging to or not belonging to the reference population. The correlation may take into account the presence or absence of one or more tRNA^{HisGTG} fragments in a test sample and the frequency of detection of the tRNA^{HisGTG} fragments in a test sample compared to a control. The correlation may take into account both of such factors to facilitate a diagnosis of a disease or condition. In certain embodiments, the reference is the identity and abundance level of the tRNA^{HisGTG} fragment present in a control sample, such as non-diseased cell, a cell obtained from a patient that does not have the disease or condition at issue or a propensity to develop such a disease or condition. In other embodiments, the reference is a baseline level of the tRNA^{HisGTG} fragment presence and abundance in a biologic sample derived from the patient prior to, during, or after treatment for the disease or condition. In yet other embodiments, the reference is a standardized curve.

### Methods of Use

The method described herein includes diagnosing, identifying or monitoring a disease or condition, such as breast cancer, in a subject in need of therapeutic intervention. In certain embodiments, the method includes isolating tRNA^{HisGTG} fragments from a cell, tissue or body fluid obtained from the subject; hybridizing the tRNA^{HisGTG} fragments to a panel of oligonucleotides engineered to detect the tRNA^{HisGTG} fragments; analyzing an identity and levels of the tRNA^{HisGTG} fragments present in the cell; wherein a differential in the identity or measured tRNA^{HisGTG} fragment levels to the reference is indicative of a diagnosis or identification of breast cancer in the subject; and providing a treatment regimen to the subject dependent on the differential in the identity and measured tRNA^{HisGTG} fragment levels to the reference. The tRNA fragments may be isolated by a method known in the art or selected from the group consisting of size selection, sequencing, amplification. The tRNA fragments may be quantified by a method known in the art or selected from dumbbell-PCR, FIREPLEX®, miR-ID®, or related. In some embodiments,^{HisGTG} tRNA fragments in the range of about 10 nucleotides to about 80 nucleotides are isolated. The range of sizes may include, but is not limited to, from about 15 nucleotides to about 55 nucleotides, and from about 17 nucleotides to about 52 nucleotides. The size of the tRNAs may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 nucleotides.

The signature is a tRNA^{HisGTG} fragment profile that comprises the identity, abundance and relative abundance of tRNA^{HisGTG} fragments. The tRNA^{HisGTG} fragment location within the genomic loci of origin, the starting and ending points of the tRNA fragment, the length of the tRNA fragment, and any other feature of the tRNA fragment as compared to other tRNAs within the same sample or another sample or reference may be included in the HisGTG tRNA fragment signature. In certain embodiments, the signature is obtained by hybridization to a single oligonucleotide, or to a panel of oligonucleotides, such as those that comprise at least two or more oligonucleotides that selectively hybridize to the tRNA fragments. To prepare the sample for characterization, the tRNA fragments and tRNA^{HisGTG} fragments may be amplified prior to the hybridization.

The therapeutic methods (which include prophylactic treatments) to treat a disease or condition, such as a disease selected from the group consisting of a cancer, and genetically predisposed disease, in a subject include administering a therapeutically effective amount of an agent or therapeutic to a subject (e.g., animal, human) in need thereof, including a mammal, particularly a human. Such treatment will be suitably administered to subjects, particularly humans, suffering from, having, susceptible to, or at risk for the disease or condition or a symptom thereof. The agent may be identified in a screening using tRNA signatures or relative abundance of tRNAs in in vitro or in vivo animal model for the disease or condition.

### Monitoring

Methods of monitoring subjects that are at high risk of developing a disease or condition, or are at risk of disease or condition recurrence, or who are receiving therapeutic intervention to reduce, improve, or treat a symptom of the disease or condition, such as breast cancer, are also useful in determining whether to administer treatment and in managing treatment. Provided are methods where the tRNA^{HisGTG} fragments are measured and characterized. In some cases, the tRNA^{HisGTG} fragments are measured and characterized as part of a routine course of action. In other cases, the tRNA^{HisGTG} fragments are measured and characterized before and again after subject management or treatment. In these cases, the methods are used to monitor the onset of a disease or condition, the recurrence of the disease or condition, the status of the disease or condition, or a propensity to develop such disease or condition, e.g., breast cancer.

For example, characterization of tRNA^{HisGTG} fragments or signatures can be used to monitor a subject's response to certain treatments. Such characterization can be used to monitor for the presence or absence of the disease or condition. The changes in the relative abundance or tRNA signature delineated herein before treatment, during treatment, or following the conclusion of a treatment regimen may be indicative of the course of the disease or condition, progression of disease or condition, or response to treatment. In some embodiments, characterization of HisGTG tRNA fragments or signatures may be assessed at one or more times (e.g., 2, 3, 4, 5). Analysis of the tRNA^{HisGTG} fragments are made, for example, using a size selection, amplification, and sequencing, or other standard method to determine the tRNA^{HisGTG} fragment profile. If desired, a tRNA^{HisGTG} fragment profile is compared to a reference to determine if any alteration in the tRNA^{HisGTG} fragment profile is present. Such monitoring may be useful, for example, in assessing the efficacy of a particular treatment in a patient. Therapeutics that normalize the tRNA^{HisGTG} fragment profile are taken as particularly useful.

### Kits

Kits for diagnosing, identifying or monitoring a disease or condition, such as breast cancer, are included. In one aspect, the invention includes a panel of engineered oligonucleotides comprising a mixture of oligonucleotides that are about 15 to about 50 nucleotides (nts) in length and capable of hybridizing tRNA fragments and tRNA^{HisGTG} fragments, wherein the tRNAs and tRNA^{HisGTG} are less than about 80 nts in length. In another aspect, the panel of engineered oligonucleotides hybridizes to at least one tRNA^{HisGTG} fragment comprising SEQ ID NOs: 1-858. In another aspect, the invention includes a kit for high-throughput analysis of tRNA fragments or tRNA^{HisGTG} fragments in a sample comprising the panel of engineered oligonucleotides of the present invention; hybridization reagents; and tRNA fragment isolation reagents. In some embodiments, the kit could include: a specially designed TaqMan® Gene Expression Assays, TaqMan® Low Density Array-micro fluidic cards; a set of end-point specific assays such as dumbbell-PCR; a set of miR-ID assays. Other kits with variations on the components and oligonucleotide panels may be used in the context of the present invention. For example, the panel of engineered oligonucleotides may be specific to a cell type, disease type, stage of disease, or other aspect that may differentiate tRNA fragment signatures. The kits and oligonucleotide panel may also be used to identify agents that modulate disease, or progression of disease in in vitro or in vivo animal models for the disease.

The practice of the present invention employs, unless otherwise indicated.

conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual,” fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The Results of the experiments disclosed herein are now described.

### Example 1: The HisGTG tRNA Locus

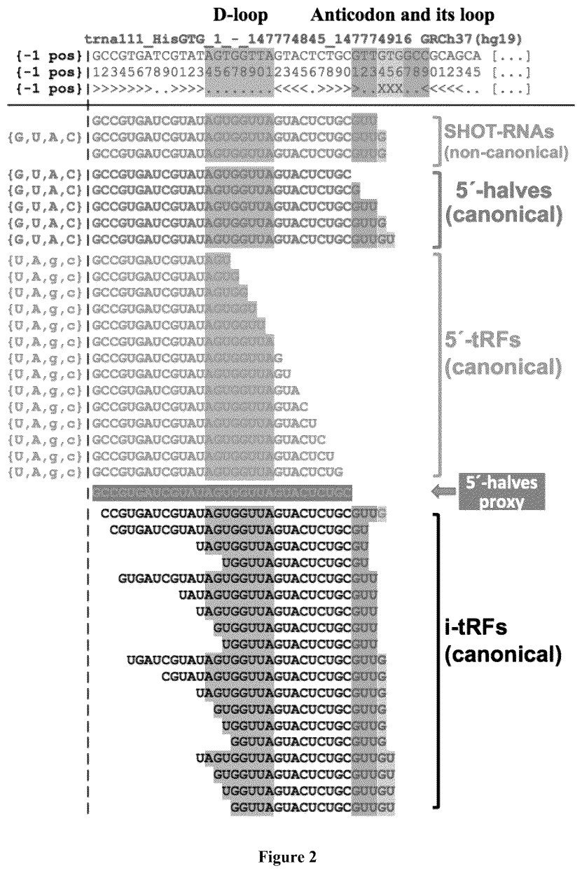

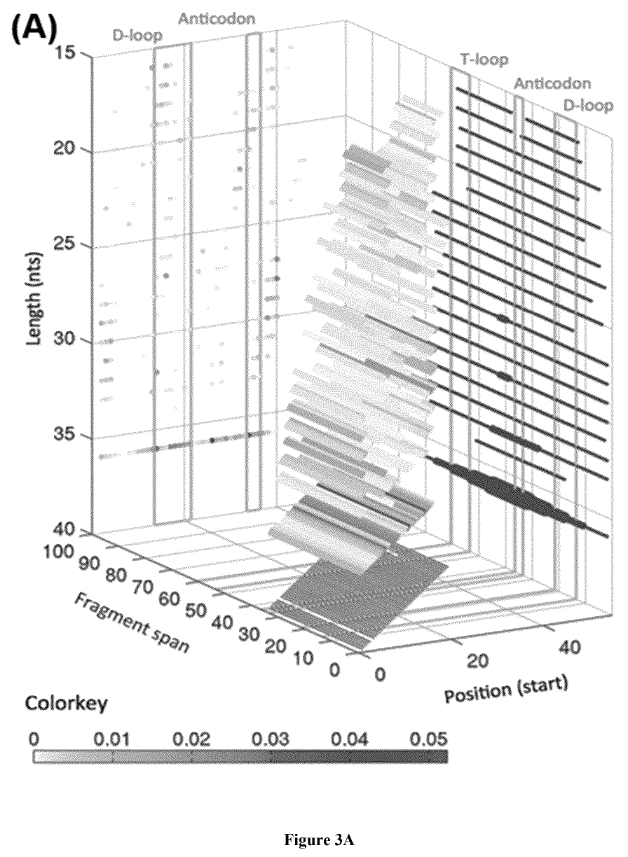

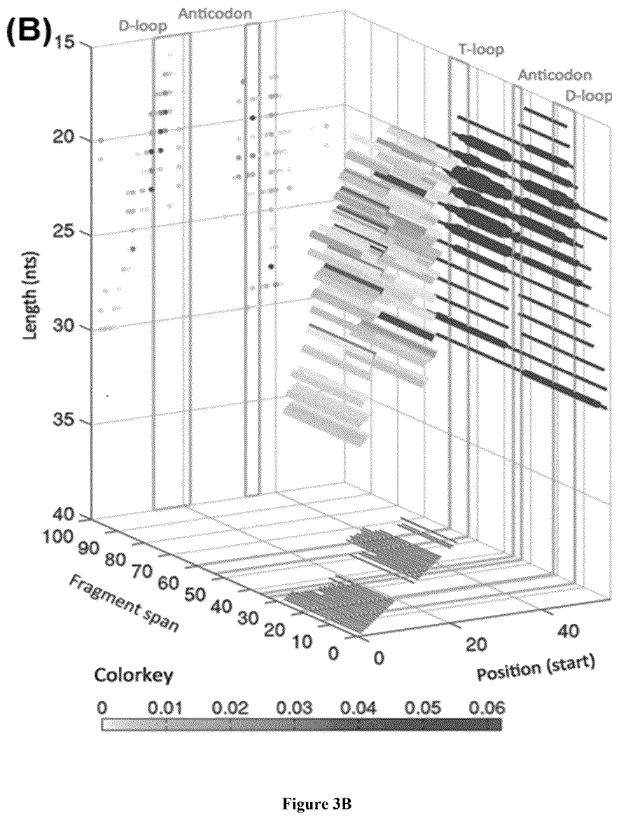

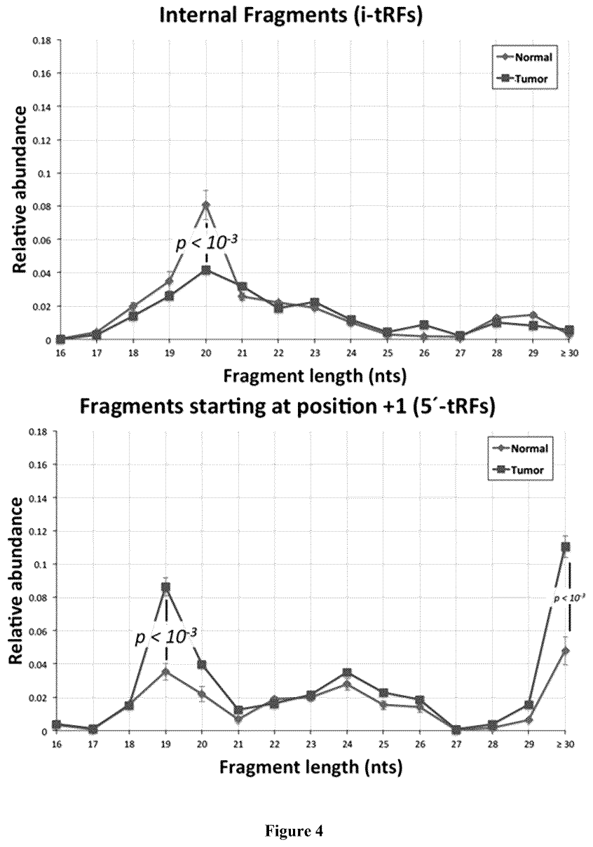

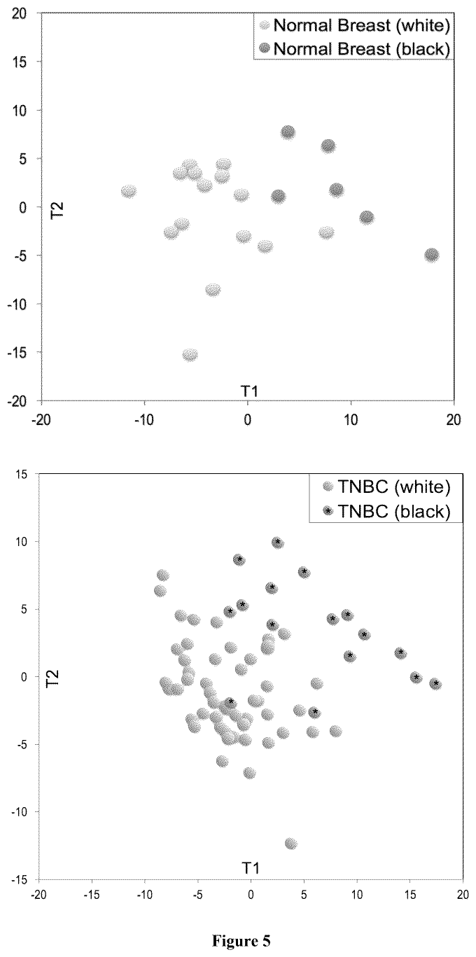

tRFs arising from the nuclear tRNA^{HisGTG} locus (also referred to as tRNAHisGTG locus) are of particular interest in the present invention. tRFs from this and other tRNA loci are present in hundreds of transcriptomes from two different human tissues, in healthy individuals and in cancer patients. tRFs from this and other tRNA loci were also shown to be produced constitutively in cells. FIG. 2 shows several tRFs from the tRNA^{HisGTG} locus aligned against the sequence of the mature tRNA of the tRNA^{HisGTG} isodecoder located on chromosome 1 between locations 147774845 and 147774916 (hg19/GRCh37 human genome assembly). The results listed below herein extend further the analyses of fragments from the nuclear tRNA^{HisGTG} locus to the subset of 10,274 normal and disease samples of the Cancer Genome Atlas (TCGA) repository whose records were not marked for withdrawal by the various TCGA consortia analyzing the different cancer types. The tRFs considered in the present invention are the ones whose sequences overlap a mature tRNA.

### Example 2: Mining tRFs in The Cancer Genome Atlas (TCGA)

Profiling tRFs that may be present in a deep-sequencing tRNA-seq) dataset is unlike the case of miRNAs miRNAs. Similarly to tRNA fragment studies, one must map on the full genome because mapping RNA-seq reads on only the several hundred isodecoders present in the nuclear and MT genomes will generate false positives. This problem is particularly acute given a report of hundreds of lookalikes of nuclear and mitochondrial tRNAs in the nuclear genome. Mapping on tRNA space alone will miss the fact that some reads map to both true tRNAs and non-tRNA space and should be discarded. Moreover, to avoid localization errors, tRF mapping must be exact and not permit replacements or indels. The nuclear genome contains multiple instances of tRNA isodecoders, tRNA lookalikes, and partial tRNA sequences, and multi-mapping will ensure an exhaustive enumeration of genomic sources and the discarding of reads that map to tRNAs and elsewhere. To accommodate fragments from the 31 tRNAs that contain introns, one must allow reads to span exon-exon junctions, and discard reads that partially step on the intron at these loci. Finally, one need to accommodate reads that extend to the non-templated “CCA” that is added post-transcriptionally to the 3′-terminus of all mature tRNAs.

An additional consideration is that of deciding a threshold above which a sequenced RNA is viewed as non-noise. The differences in sequencing depth that are present in TCGA RNA-seq datasets require that an adaptive threshold be used. An algorithm, “Threshold-seq,” can automatically determine such a threshold and was used to pre-process each dataset and keep those tRFs that exceeded the algorithm's recommended threshold. In certain non-limiting embodiments, the present invention is restricted to fragments in the range 16-50 nt whose sequences overlap a mature tRNA. When working with short RNA-seq profiles from the TCGA repository, one needs to be mindful that in that project deep-sequencing PCR was run for 30 cycles only. In the case of tRFs, many tRFs exist that are longer than 30 nt: in analyses of TCGA data, these longer tRFs will appear truncated and will be represented by “30-mers.”

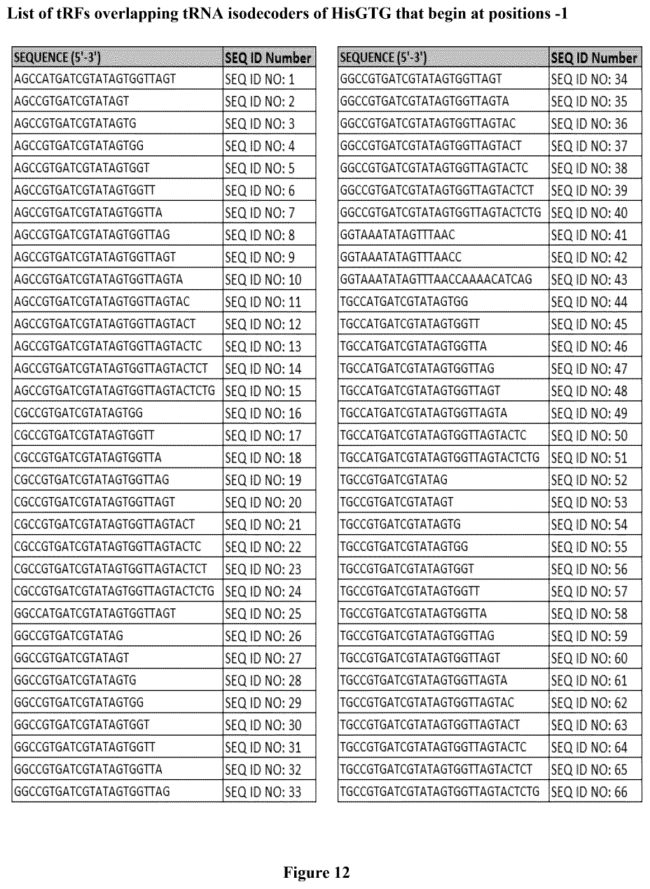

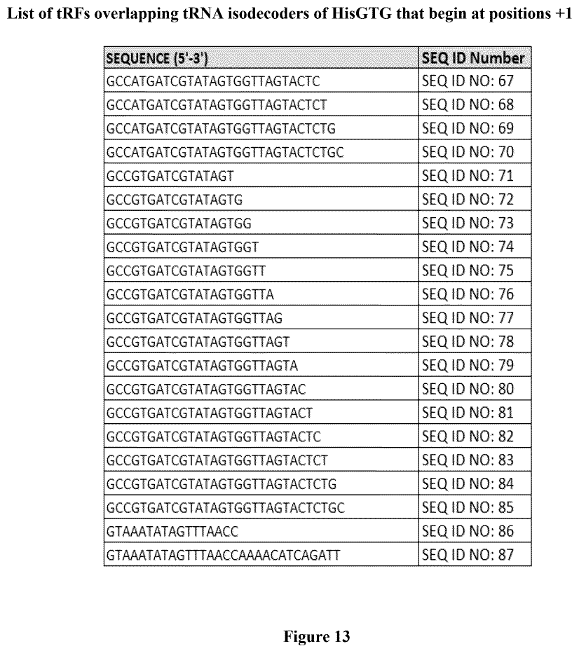

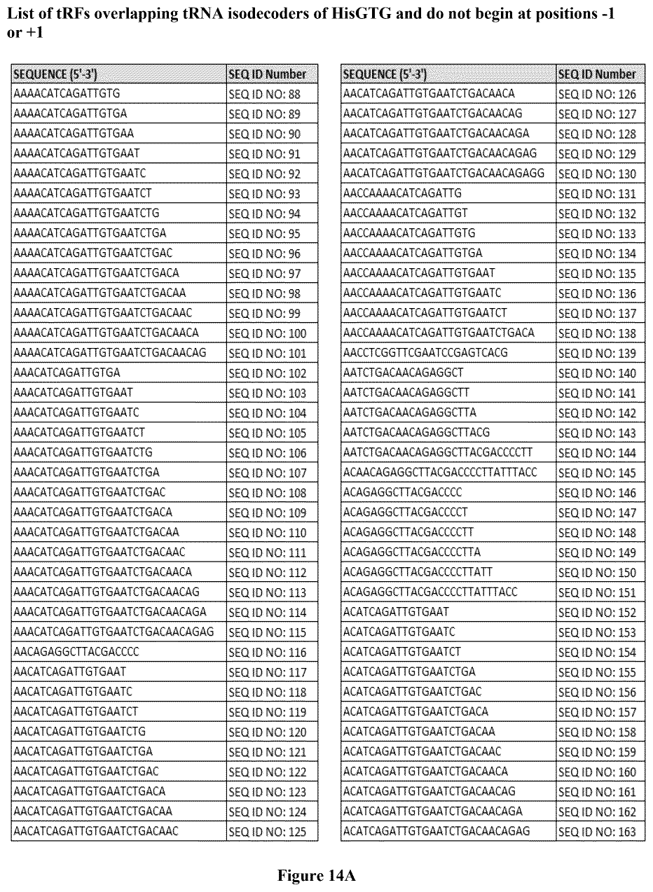

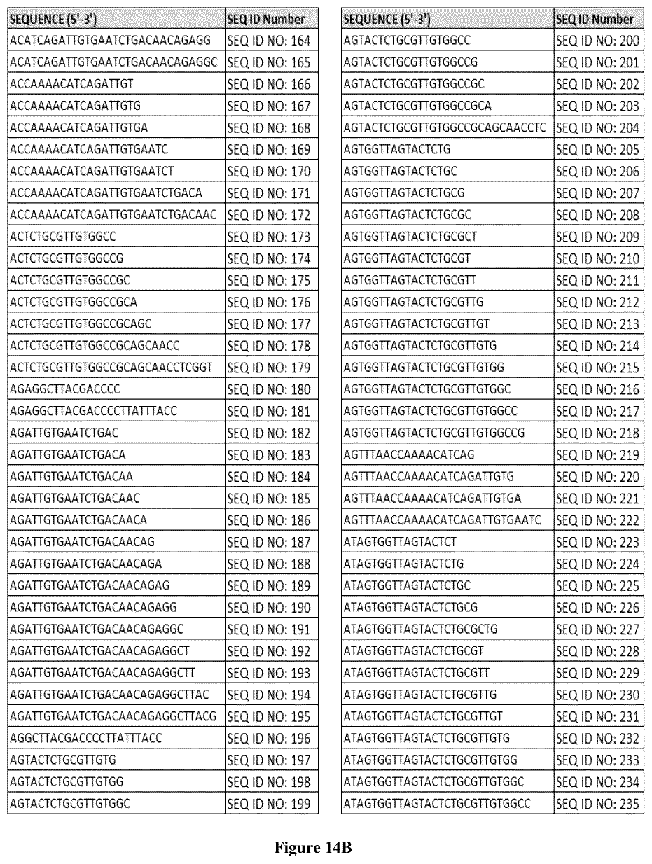

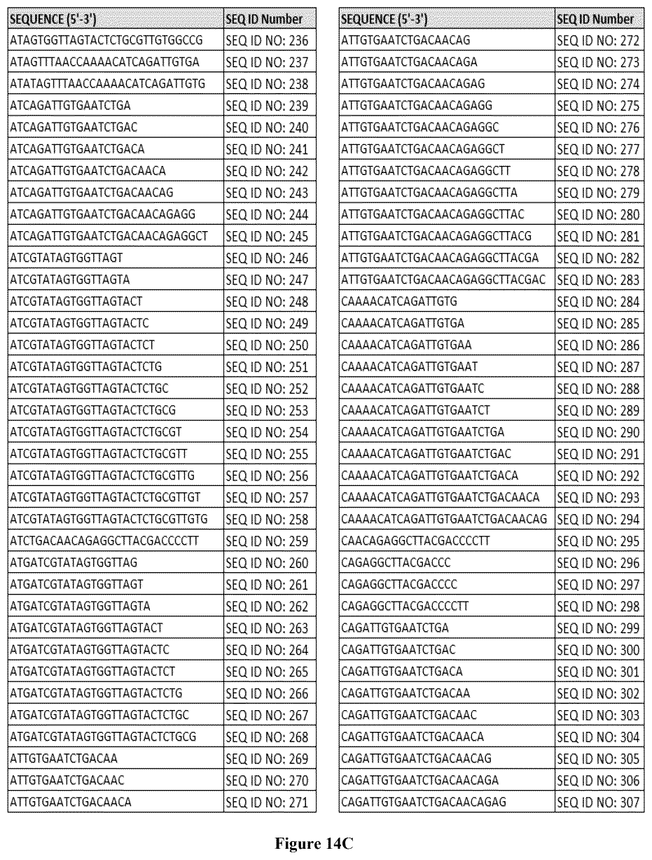

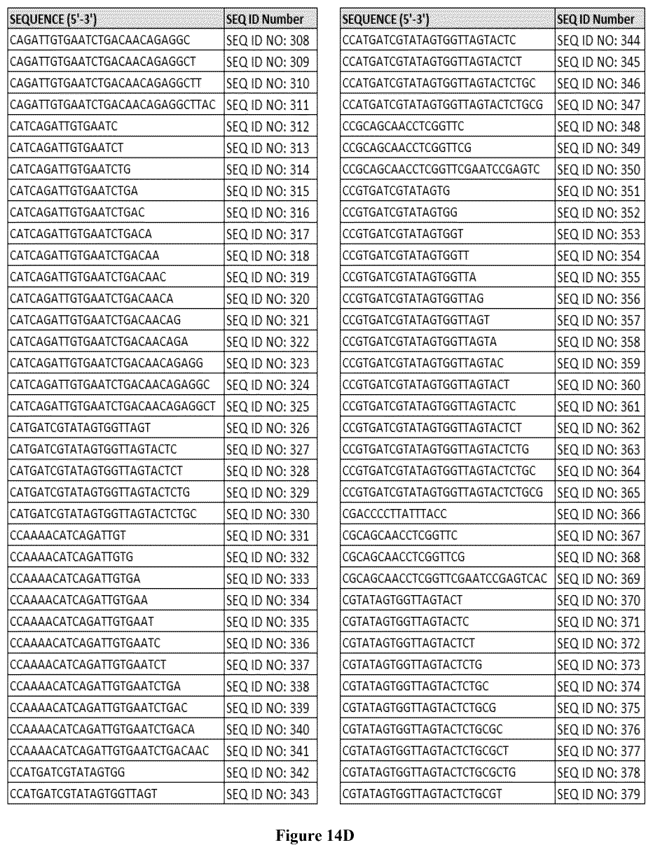

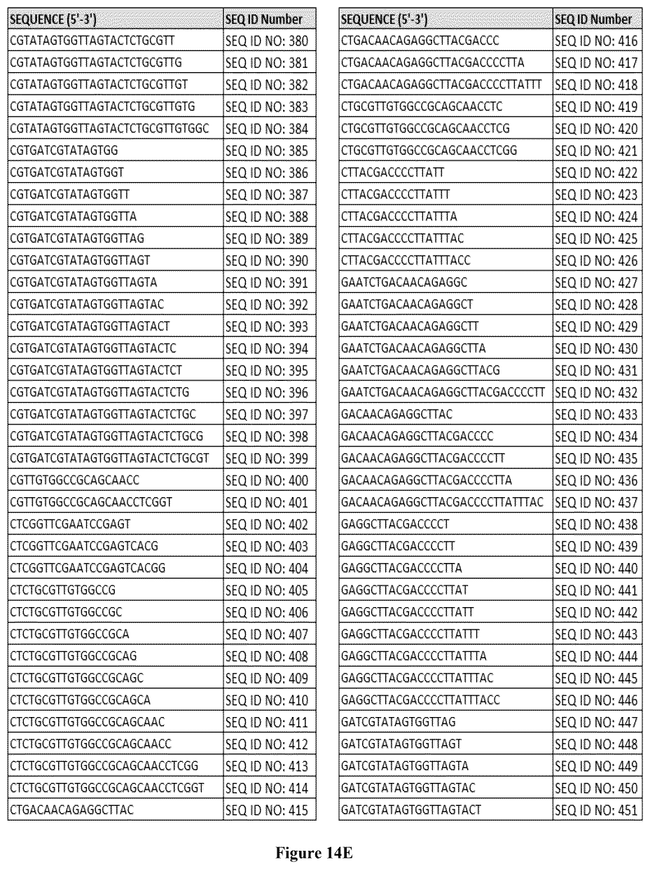

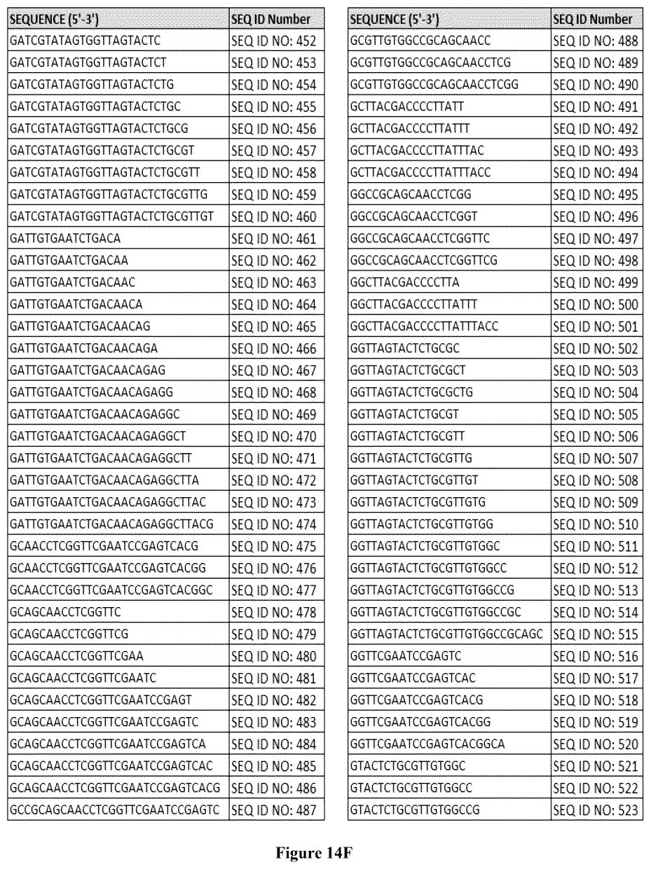

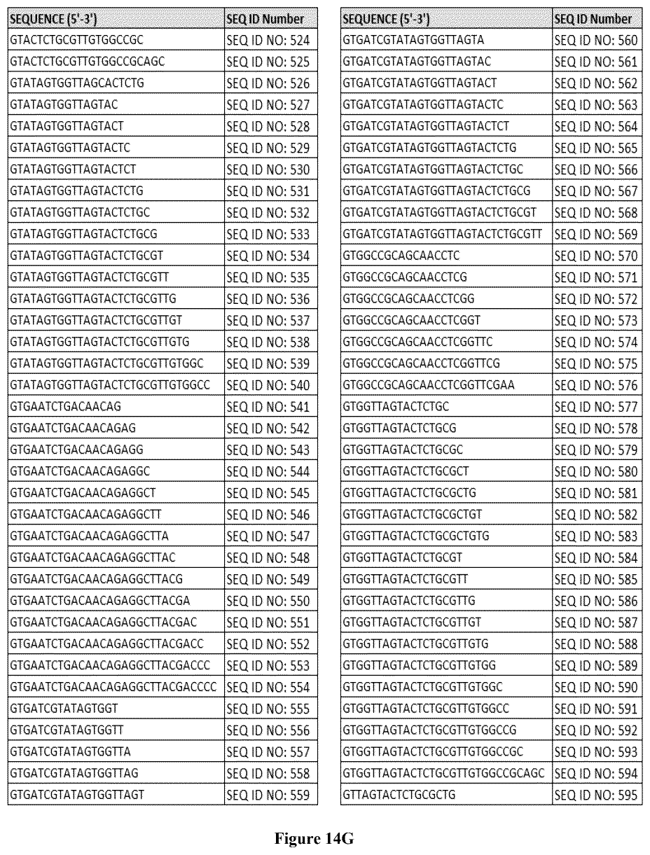

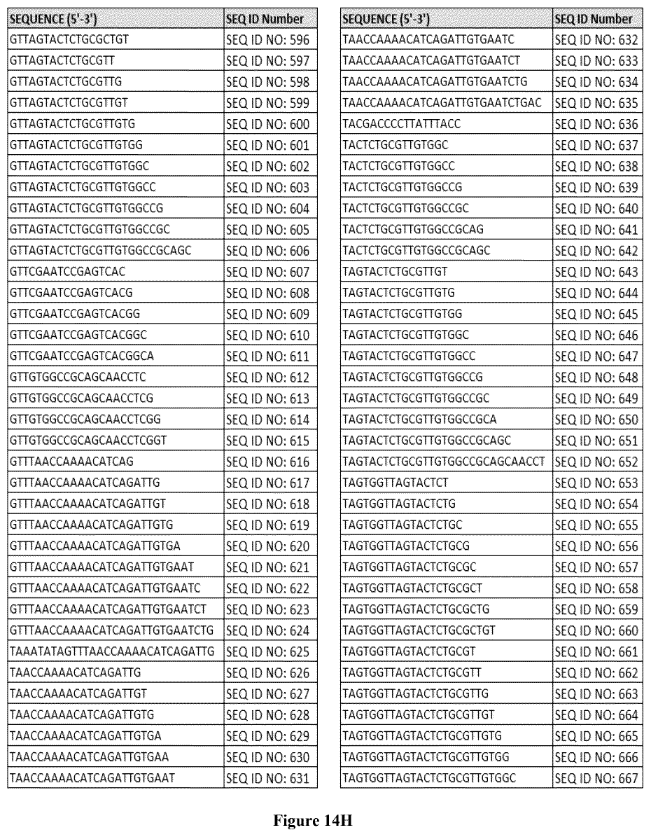

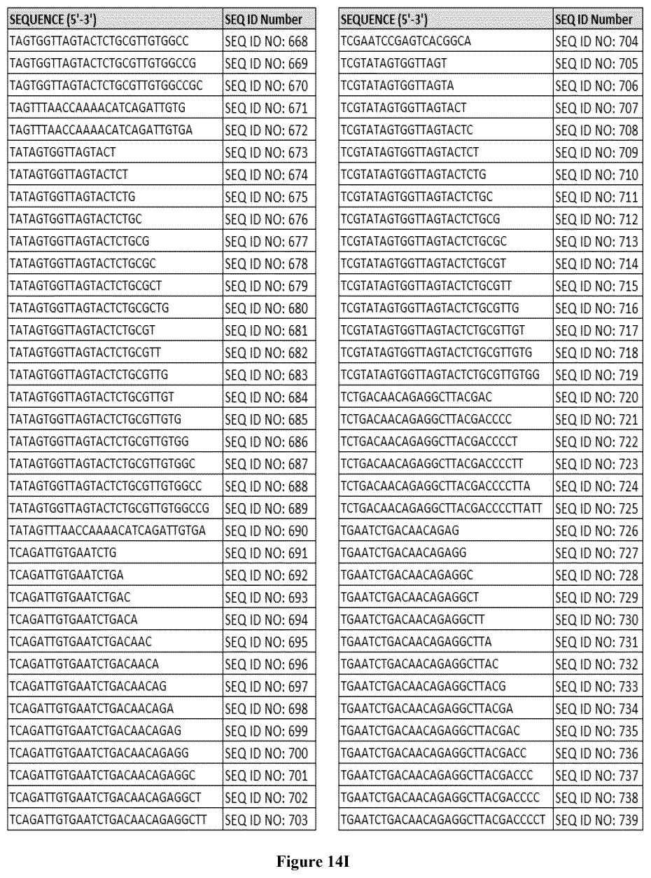

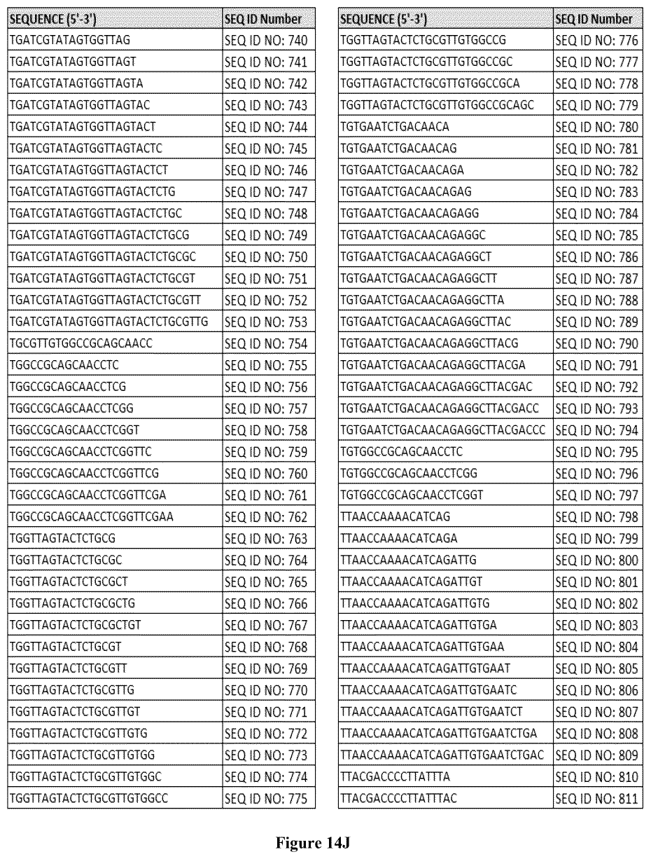

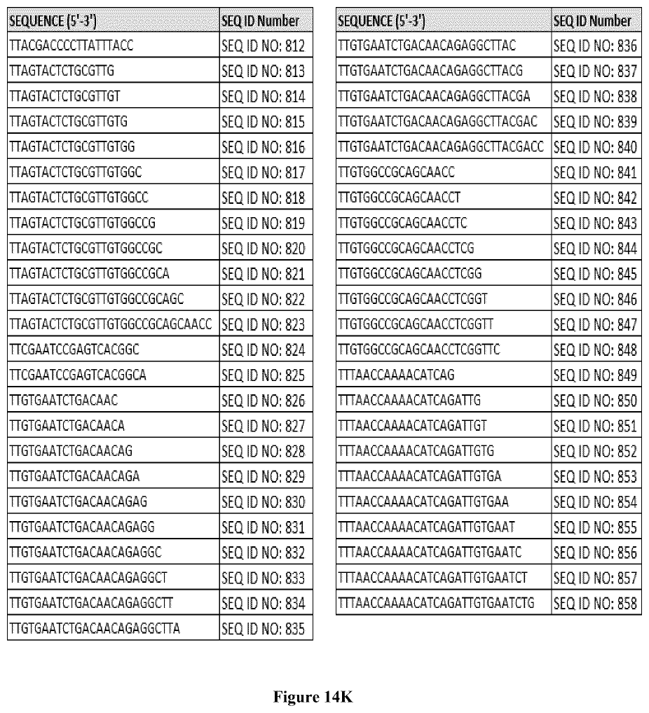

The analysis of the 10,274 datasets mentioned above herein generated 20,722 distinct tRFs above threshold. Of interest are those fragments that overlap the mature tRNA of HisGTG. Specifically, the 66 tRFs that begin at position −1 of isodecoders of tRNA^{HisGTG} (FIG. 12, SEQ ID NOs: 1-66), 21 tRFs that begin at position +1 of isodecoders of tRNAHisGTG (FIG. 13, SEQ ID NOs: 67-87), and the 771 tRFs that begin at positions other than −1 or +1 (FIGS. 14A-14K, SEQ ID NOs: 88-858).

### Example 3: Uridylated His(−1) tRFs are Abundant in Human Tissues

In eukaryotes, before the mature tRNA from tRNA^{HisGTG} can be recognized by its cognate aminoacyl tRNA synthetase, guanylation of its 5′-terminus by the enzyme THG1 (THG1L in human) is required. This post-transcriptionally added nucleotide is referred to as the “−1” position and denoted “His(−1).” Recent work with the breast cancer model cell line BT-474 showed that full-length mature tRNAs and 5′ halves from tRNA^{HisGTG} also contain a uracil at the His(−1) position (Shigematsu & Kirino, 2017, RNA, 23(2):161-168). This possibility has not been examined before in primary human tissue. The present analyses of the TCGA datasets reveal that in human tissues, and across all 32 cancer types, the largest portion of 5′-tRFs from tRNA^{HisGTG} contain a uracil at the His(−1) position (−1U 5′-tRFs). For example, in the TCGA BRCA datasets, the ratio of guanylated to uridylated fragments is approximately 1:10. A smaller fraction of 5′-tRFs contain an adenine at the His(−1) position, whereas 5′-tRFs with a guanine or cytosine are even fewer. The presence of a guanine or adenine at the −1 position suggests that these tRFs are the result of post-transcriptional enzymatic action. Indeed, the genomic sequence contains no A or G immediately upstream of the 11 nuclear and one mitochondrial isodecoders of tRNA^{HisGTG}. However, the same cannot be said of tRFs with a uracil or a cytosine at that position: four of the 12 isodecoders (the MT one and the three nuclear tRNA-His-GTG-1-6, tRNA-His-GTG-3-1, tRNA-His-GTG-1-5) contain a T at that location of the genome whereas the remaining 8 contain a C; thus, these tRFs could be either the product of post-transcriptional enzymatic action or the result of cleavage of the precursor tRNA.

### Example 4: Uridylated His(−1) tRFs Exhibit a Property that is Not Affected by Tissue or Tissue State

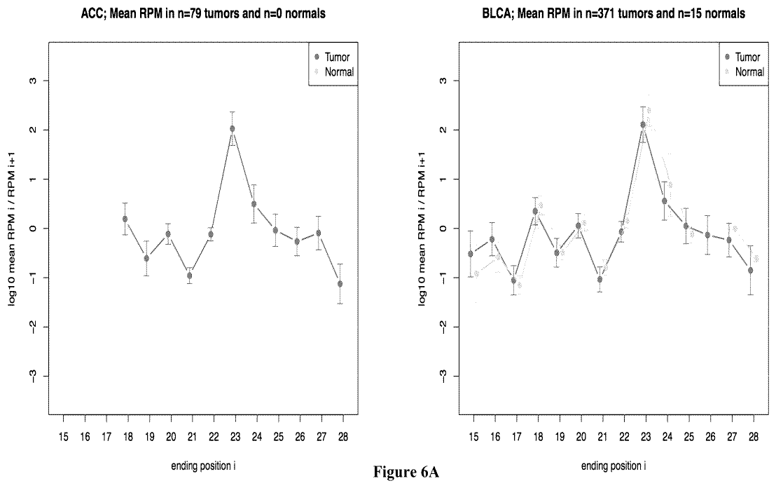

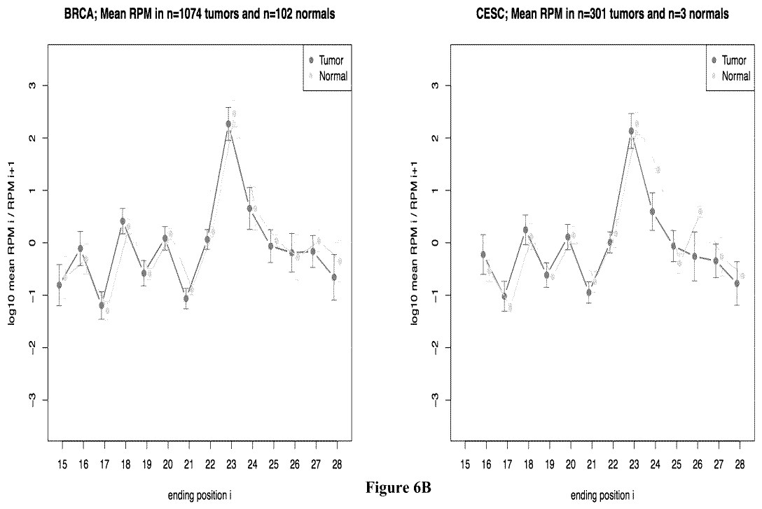

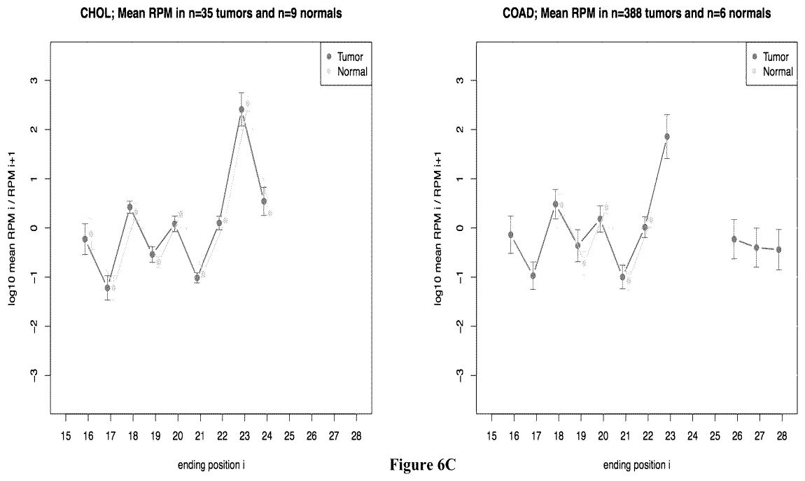

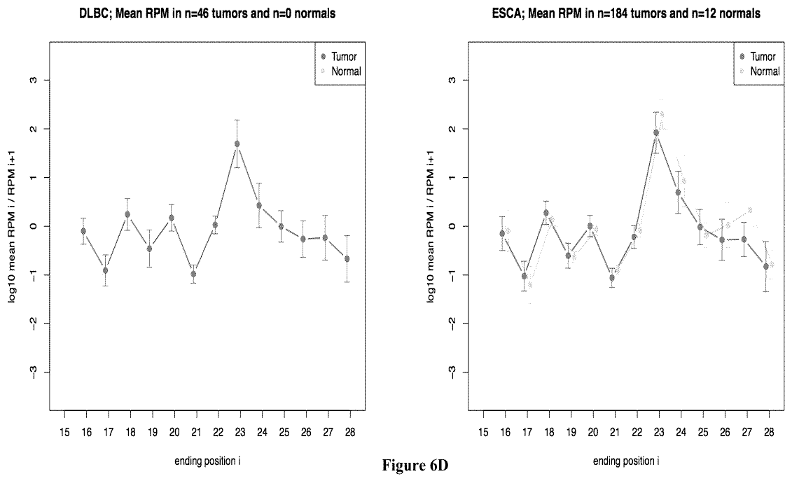

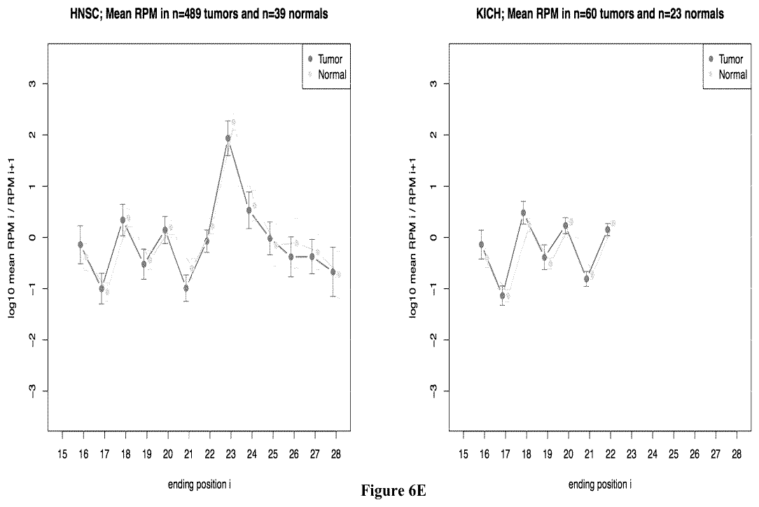

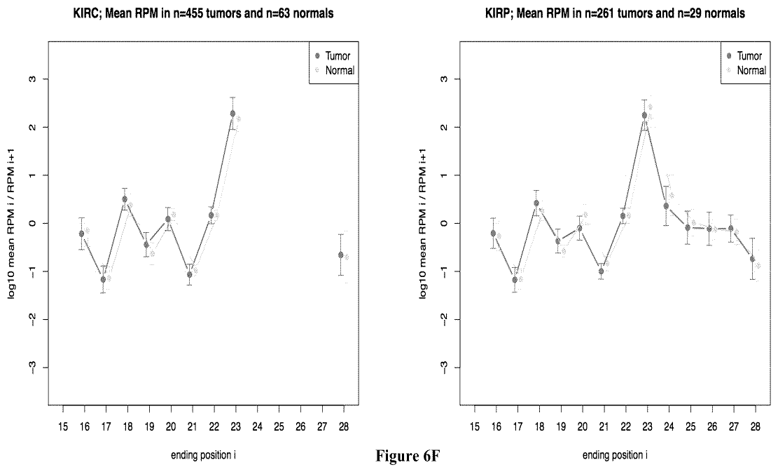

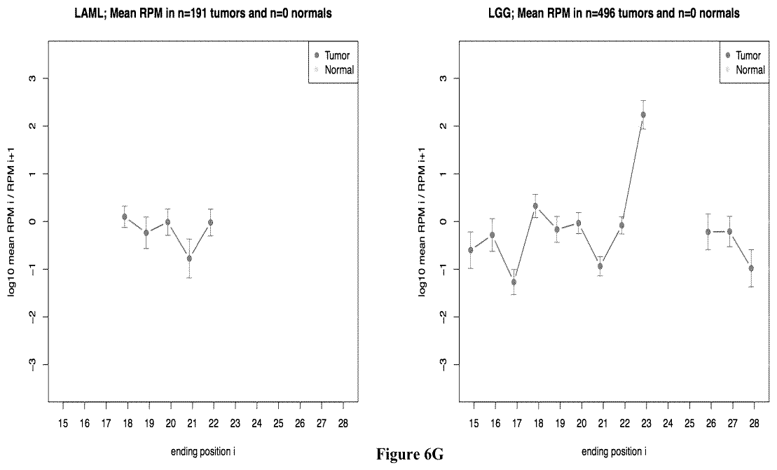

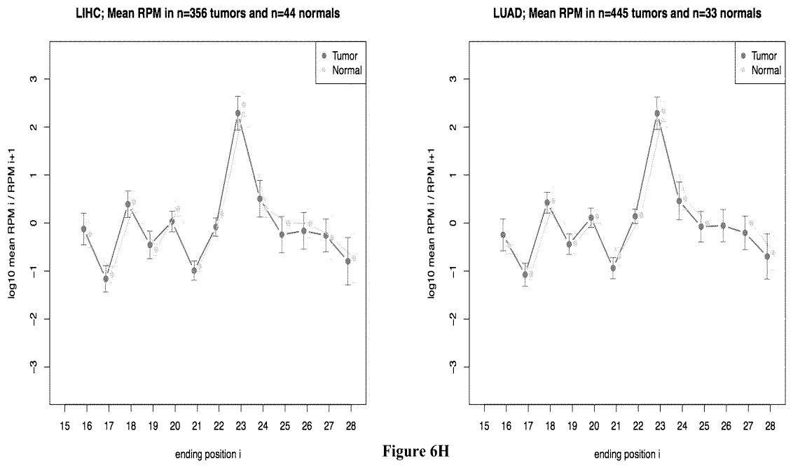

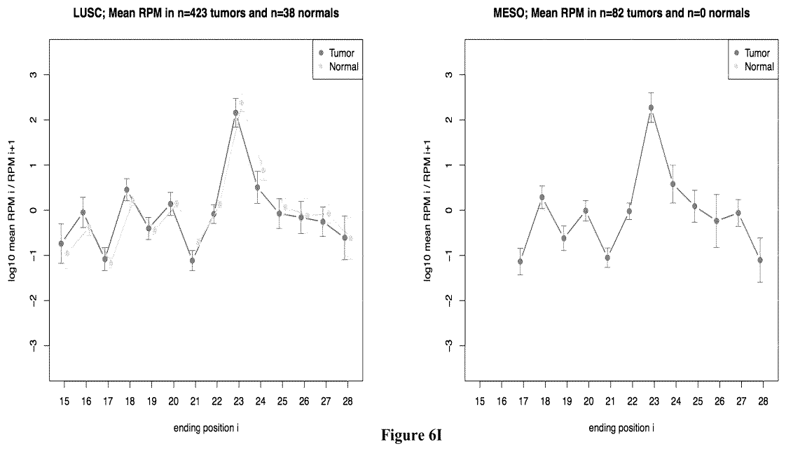

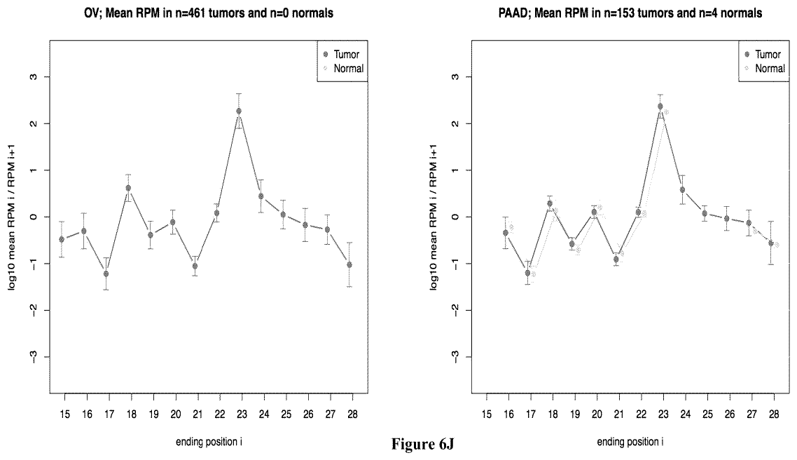

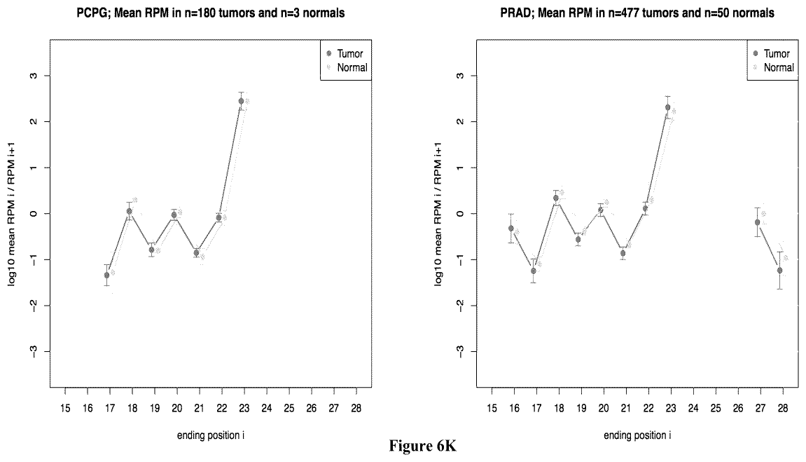

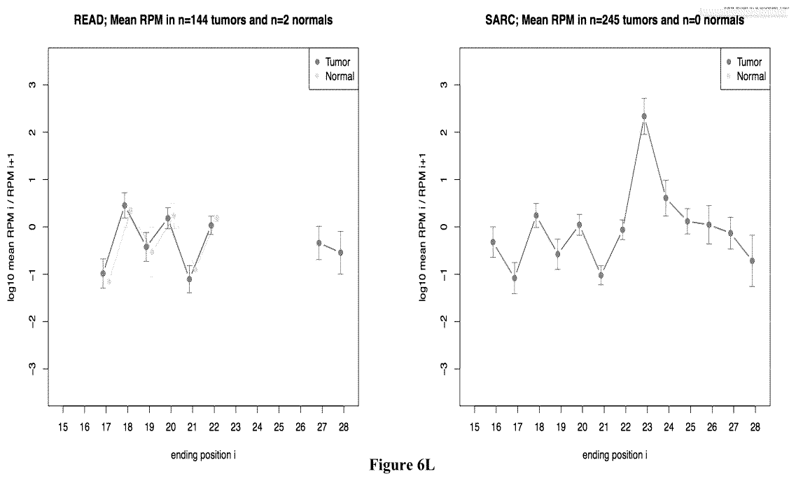

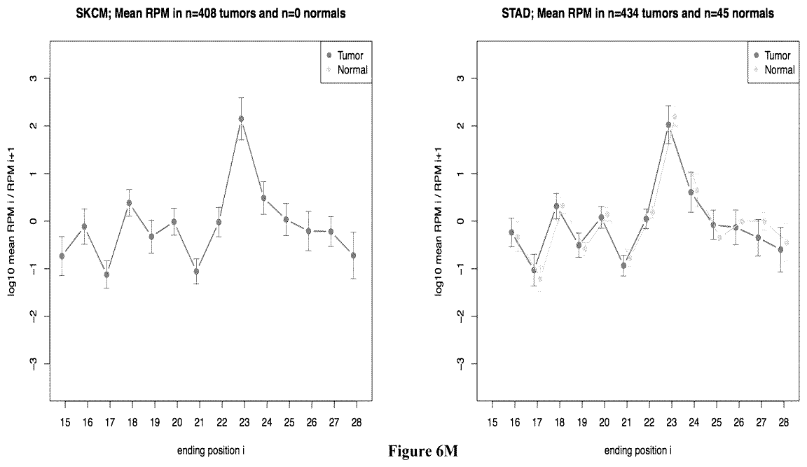

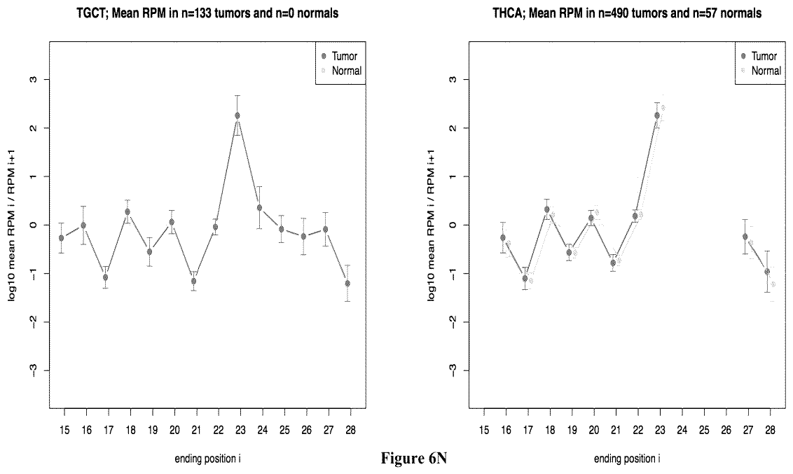

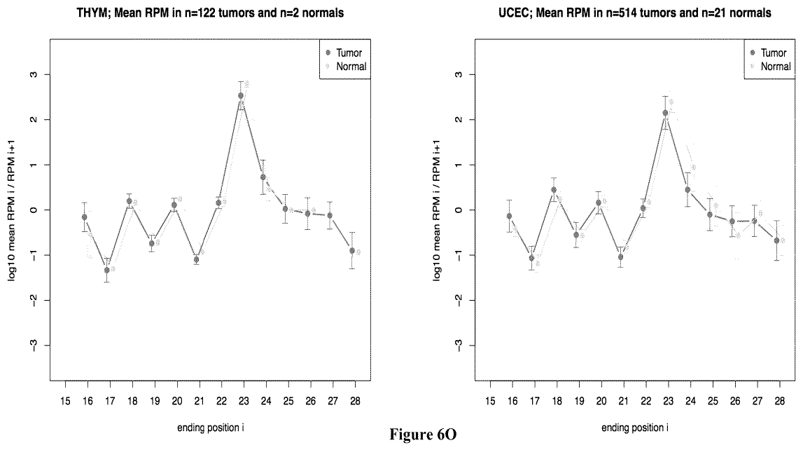

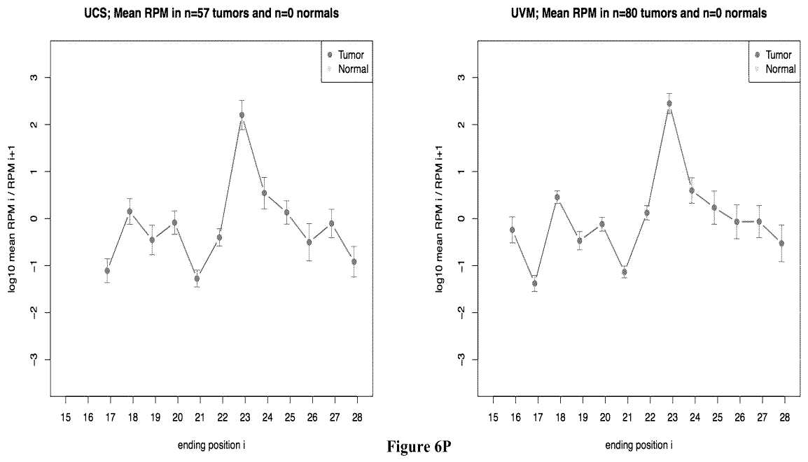

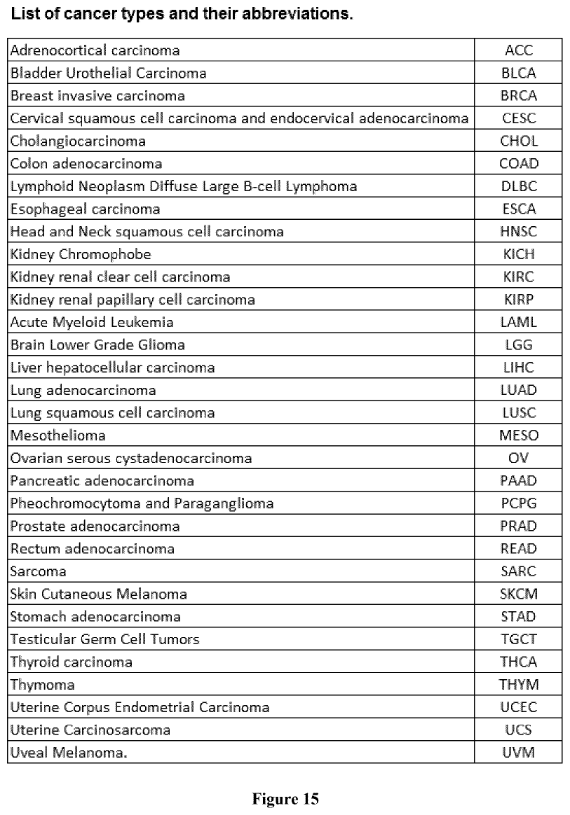

Uridylated His(−1) 5′-tRFs were examined across all 32 TCGA cancer types and uncovered an intriguing property. The property pertains to those His(−1) tRFs from tRNA^{HisGTG} that have a T (U) in their −1 position, differ by a single nucleotide in their 3′ terminus and have lengths between 16 and 25 nt inclusive. As the His(−1) tRF lengths increase, the tRFs' abundance was shown to alternate from low to high, to high to low, and so forth. More specifically, the ratio of abundances of these increasingly longer fragments remain constant in all 32 TCGA cancers. Notably the pattern remained unchanged between the normal and disease state of the tissue. FIG. 6A-6P shows the log 10 of the mean ratio of (abundance of His(−1) 5′-tRF ending at position i)/(abundance of His(−1) 5′-tRF ending at position i+1), for all 32 cancer types. The various panels of FIGS. 6A-6P follow the abbreviations shown in FIG. 15. In each sample, tRF abundances were normalized by converting them to reads-per-million (RPM) values. E.g. two such consecutive fragments are T-GCCGTGATCGTATAGT (SEQ ID NO: 54) and T-GCCGTGATCGTATAGT-G (SEQ ID NO: 55). In those cancer types for which normal samples are available, the values for both the tumor (black) and normal (grey) samples were reported. The points of the grey (black, respectively) curve are shifted slightly to right (left, respectively) along the X-axis in order to make the details of both curves visible simultaneously. This finding suggests that the biogenesis of these uridylated His(−1) 5′-tRFs is under exquisite control and that the specifics of this process are conserved across tissues, in health and disease, and across all TCGA cancer types. This conserved relationship suggests that these 5′-tRFs, whether instigators or effectors, participate in cellular process that are common to all cancer types, and, thus, of essential nature.

### Example 5: tRFs at Large are Loaded on Argonaute (Ago) in a Cell-Line-Specific Manner

tRFs can be loaded on Ago (Burroughs et al., 2011, RNA biology 8:1, 158-177; Kumar et al., 2014, BMC biology 12:1, 78; Maute et al., 2013, PNAS 110:4, 1404-1409). Ago loading, of course, suggests that at least some tRFs can enter the RNA interference tRNAi) pathway and regulate their targets through RNAi. The profile of Ago-loaded tRFs is a function of cell type (Telonis et al., 2015, Oncotarget 6:28, 24797-24822). Specifically, the public Ago HITS-CLIP datasets that were discussed in Pillai et al., 2014, Breast cancer research and treatment 146:1, 85-97 and were obtained from three breast cancer cell lines (MCF7, BT474 and MDA-MBA-231) were used herein. Through the present analysis each cell line was shown to exhibit a profile of Ago-loaded tRFs that differs from that of the other two cell lines (Telonis et al., 2015, Oncotarget 6:28, 24797-24822).

### Example 6: The Ago loading of His(−1) tRFs Depends on Cell Line and on 5′-Modification

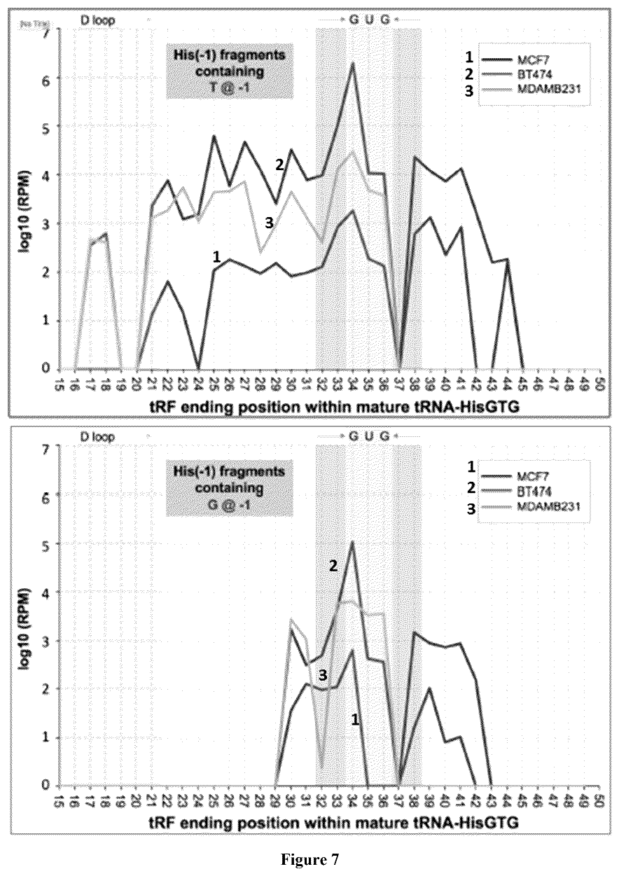

The Ago HITS CLIP-seq datasets of Pillai et al., 2014 was also examined herein specifically for instances of tRFs from tRNA^{HisGTG}. FIG. 7, top panel, shows the distribution of Ago-loaded His(−1) fragments whose −1 position has been uridylated. In particular, this figure shows the normalized abundance of His(−1) fragments that end at position “i” of the mature tRNA^{HisGTG}. With a few exceptions, the three distributions are similar qualitatively. Exceptions include: the absence in MDA-MB231 of Ago-loaded tRFs that end beyond position 36; the absence in MCF7 of Ago-loaded tRFs that end at position 24; etc.

FIG. 7, bottom panel, shows the analogous distribution for Ago-loaded His(−1) fragments whose −1 position has been guanylated. It is evident from this figure that His(−1) tRFs with a G at the −1 position exhibit different Ago-loading characteristics than those with a U at that position. Again, the MDA-MB231 cell line shows characteristic differences compared to the other two cell lines.

FIG. 7 (top and bottom panels) shows that Ago-loading pattern depends on the cell line and on the moiety that was added to the 5′-terminus. Naturally, these differences suggest a concomitant dependence of the downstream RNAi targets on the identities of these His(−1) tRFs. Lastly, His(−1) tRFs with an A occupying position −1 adenylated are also present in the analyzed HITS CLIP-seq data.

### Example 7: Non-Canonical tRF Variants

The standard RNA-seq protocol that targets short ncRNAs includes an adapter ligation step when two different adapters with known sequence are ligated to the 5′- and 3′-termini of the RNAs. These ligation reactions require that the targeted RNA substrates be of the “P/OH type” (as defined above herein to as canonical). Consequently, standard RNA-seq only targets canonical RNA substrates and, thus, could be undercounting when it comes to establishing the identities of molecules that may be present in a sample or in a cell line of interest.

The termini of ANG-generated 5′- and 3′-SHOT-RNAs belong to the P/cP and OH/aa types respectively (Honda et al., 2015, Proc Natl Acad Sci USA, 112:29, E3816-3825). Even though from a structural standpoint they belong to “tRNA halves,” SHOT-RNAs are a distinct class in that they were shown to be specifically and abundantly expressed in ER+ breast cancer and AR+ prostate cancers respectively (Honda et al., 2015, Proc Natl Acad Sci USA, 112:29, E3816-3825). Because of their terminal modifications SHOT-RNAs are non-canonical and, thus, they are “invisible” to standard RNA-seq.

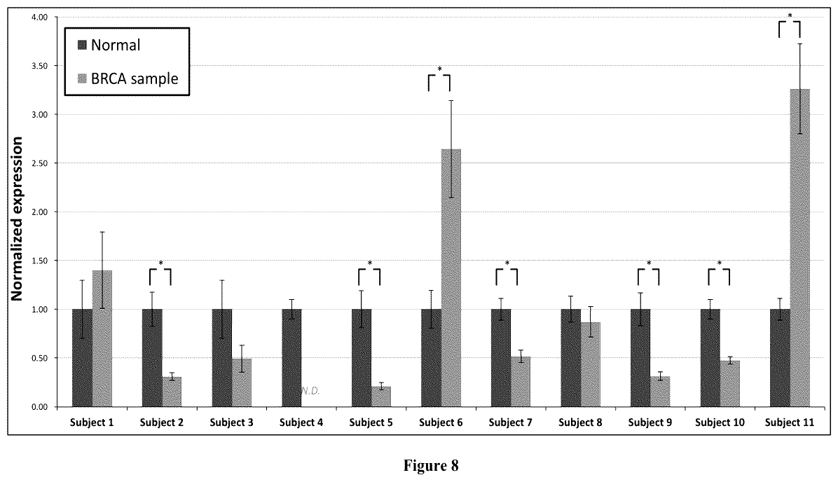

Just like SHOT-RNAs, other tRFs that are shorter than “halves” also exist in non-canonical variants. In Telonis et al., 2015 (Telonis et al., 2015, Oncotarget 6:28, 24797-24822), an i-tRF from tRNA^{AspGTC} that overlaps positions 15 through 35 inclusive of the mature tRNA, denoted AspGTC|15.35.21 here. To this end “dumbbell-PCR,” an endpoint-specific method (Honda et al., 2015, Nucleic acids research 43:12, e77), was used. 11 pairs of fresh breast tumor and adjacent normal breast tissue were tested and AspGTC|15.35.21 was found in 21 of the 22 tests (FIG. 8). AspGTC|15.35.21 was also quantitated after treatment with T4 PNK (T4 PNK turns the terminal structures of all present tRNA fragments into the P/OH type in preparation for adapter ligation) and an increase of the signal between 10× and 100× was found in all the normal breast and breast cancer samples that were tested. This indicated that AspGTC|15.35.21 also exists in variants that are abundant and are not of the P/OH type.

### Example 8: Canonical and Non-Canonical Instances of tRFs from tRNAHisGTG are Present in Model Cell Lines

The experiments listed above herein with the i-tRF AspGTC|15.35.21 in untreated and T4 PNK-treated normal breast and breast cancer samples provided first evidence that the tRF exists in two variants, canonical (P/OH type) and non-canonical.

To test if this might be true for other tRFs and other isodecoders/isoacceptors, a pilot study was carried out. This study profiled untreated total RNA from the BT-20 and MDA-MB-468 cell lines, and also total RNA that had been deacylated and treated with T4 PNK before adapter ligation. The BT-20 and MDA-MB-468 were selected herein because of the importance of these two cell lines as model for triple negative breast cancer (TNBC).

These experiments allowed verifying that many of the tRFs from tRNA^{HisGTG} and other anticodons that were identified previously as important in TNBC in particular, and in breast cancer in general, were also endogenously present in the model cell lines. More importantly, the tRFs from from tRNA^{HisGTG} and other anticodons were found to exist simultaneously as canonical (P/OH type) and also as non-canonical variants. The results found herein indicate that isodecoders of this particular isoacceptor produce many more distinct molecules than have been seen with the help of standard RNA-seq.

### Example 9: Correlations and Anti-Correlations

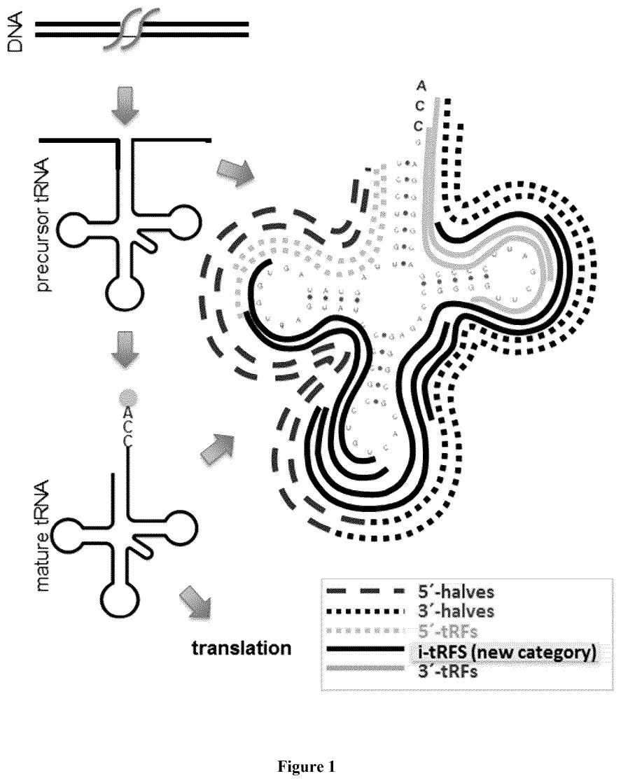

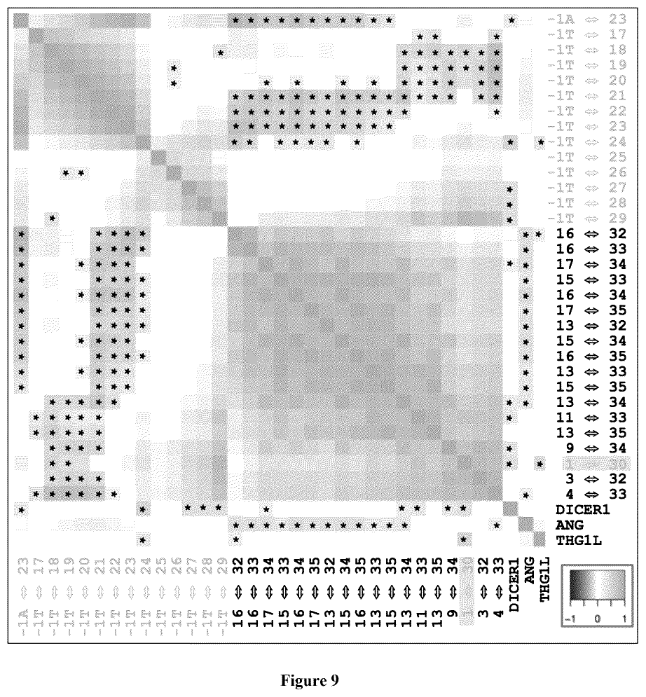

The tRFs used in this particular example are shown aligned against tRNA^{HisGTG} in FIG. 1. For the canonical tRFs among them (i.e., P/OH-type fragments) pair-wise Pearson correlations were computed in 1,049 TCGA BRCA datasets. In normal breast, in breast cancer, and across breast cancer subtypes, the guanylated His(−1) tRFS (grey labels in FIG. 9) exhibited correlated abundances. Similarly, the i-tRFs (black labels in FIG. 9) were also correlated. However, as can be seen from this figure the abundance levels of His(−1T) tRFs and i-tRFs were not correlated. In fact, for some pairings the corresponding tRFs were anticorrelated (these are indicated by asterisks “*” in the Figure). By tapping into the abundance levels of the messenger RNAs (mRNAs) of the same samples, the following was also found:

ANG mRNA is correlated with several His(−1T) tRFs and anti-correlated with several i-tRFs from the same isoacceptor; and,

DICER1 mRNA is anticorrelated with the longer among the His(−1T) tRFs and with the longer among the i-tRFs from the same isoacceptor.

### Example 10: A His(−1T) tRF and an i-tRF From the Same Isodecoder Target Different mRNAs

Two tRFs from tRNA^{HisGTG} were used herein. The first was a 23-nt-long uridylated His(−1) ending at position 22 of the mature tRNA (denoted HisGTG|−1T.22.23). The second was a 22-nt-long i-tRF that spans positions 13 through 34 inclusive of the same mature tRNA (denoted HisGTG|13.34.22). Analysis of a publicly available Ago HITS CLIP-seq data (Pillai et al., 2014, Breast cancer research and treatment 146:1, 85-97) from three breast cancer cell lines (MCF7, BT474 and MDA-MB-231) showed that both molecules are loaded on Ago and thus function in the RNAi pathway. These three cell lines serve as models for the three breast cancer subtypes, ER+, HER2+ and TNBC respectively. Two model cell lines were used, BT-20 and MDA-MB-468, both of which model TNBC, like MDA-MB-231.

Each tRF and a control (a random string of the same length and G/C content) were over-expressed, in triplicate, in the two cell lines, followed by RNA-seq profiling of all mRNAs and long ncRNAs in these cell lines.

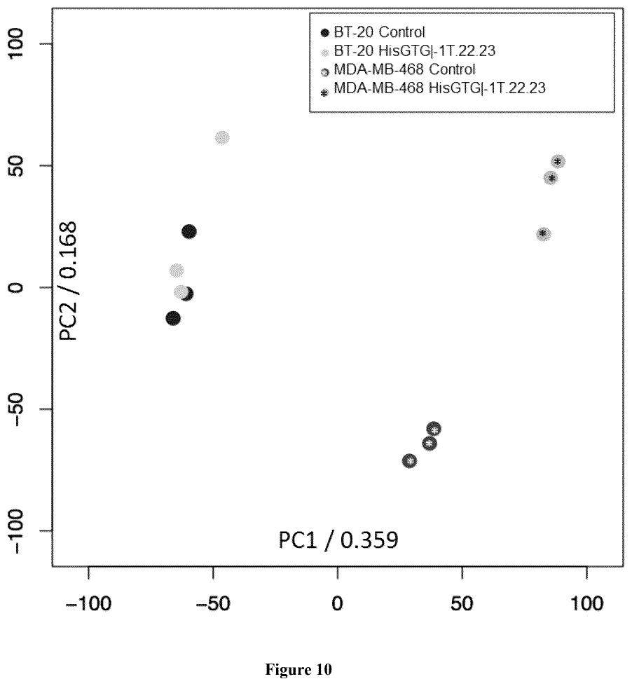

FIG. 10 shows a principal component analysis (PCA) of the transfection with HisGTG|−1T.22.23. As can be seen, this tRF had a considerable impact on mRNAs and lncRNAs in the MDA-MB-468 cell line compared to control. Differential expression analysis identified many mRNAs and lncRNAs that were differentially present following each tRF transfection, compared to control. These mRNAs and lncRNAs comprised both down-regulated and up-regulated transcripts.

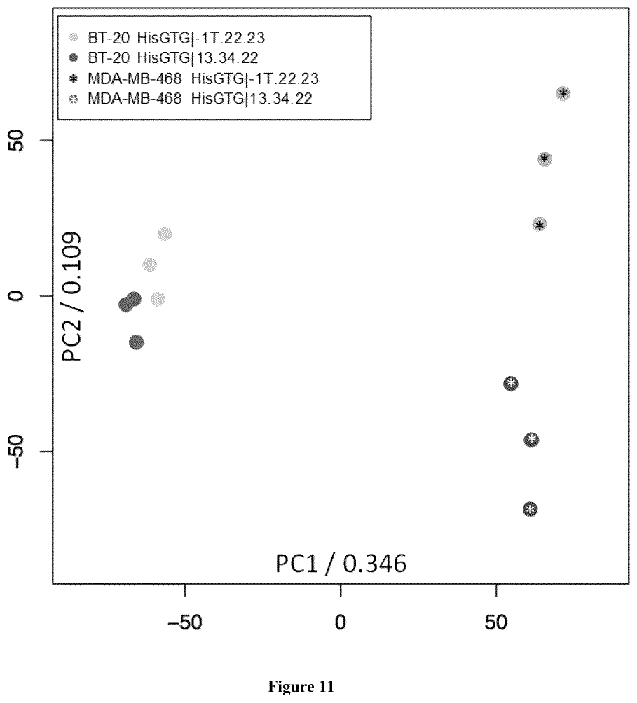

FIG. 11 compares the impact of the two tRF transfections in the two cell lines with one another. The MDA-MB-468 cell line again exhibited a more pronounced difference in response to the transfections with the HisGTG|−1T.22.23 and HisGTG|−13.34.22 respectively. In BT-20, 217 mRNAs and 267 non-coding RNAs were up-regulated following the HisGTG|−1T.22.23 transfection compared to the HisGTG|13.34.22 transfection. The 217 mRNAs included members of the following GO term categories: GO:0006753-nucleoside phosphate metabolic process, GO:0009117-nucleotide metabolic process, GO:0009891-positive regulation of biosynthetic process, GO:0010467-gene expression, GO:0010468-regulation of gene expression, GO:0010557-positive regulation of macromolecule biosynthetic process, GO:0010628-positive regulation of gene expression, GO:0016070-RNA metabolic process, GO:0019219-regulation of nucleobase-containing compound metabolic process, GO:0022900-electron transport chain, GO:0022904-respiratory electron transport chain, GO:0031328-positive regulation of cellular biosynthetic process, GO:0034645-cellular macromolecule biosynthetic process, GO:0042773-ATP synthesis coupled electron transport, GO:0042775-mitochondrial ATP synthesis coupled electron transport, GO:0045893-positive regulation of transcription, DNA-templated, GO:0045935-positive regulation of nucleobase-containing compound metabolic process, GO:0051171-regulation of nitrogen compound metabolic process, GO:0051173-positive regulation of nitrogen compound metabolic process, GO:0051252-regulation of RNA metabolic process, GO:0051254-positive regulation of RNA metabolic process, GO:0055086-nucleobase-containing small molecule metabolic process, GO:0055114-oxidation-reduction process, GO:1901566-organonitrogen compound biosynthetic process, GO:1902680-positive regulation of RNA biosynthetic process, and GO:1903508-positive regulation of nucleic acid-templated transcription. In MDA-MB-468, 109 mRNAs and 164 non-coding RNAs were up-regulated following the HisGTG|−1T.22.23 transfection compared to the HisGTG|13.34.22 transfection. The 109 mRNAs included members of the following GO term categories: GO:0006323-DNA packaging, GO:0010033-response to organic substance, GO:0007565-female pregnancy, GO:0071103-DNA conformation change, GO:0006970-response to osmotic stress, and GO:0044706-multi-multicellular organism process.

### Example 11: His tRFs and Correlated mRNAs

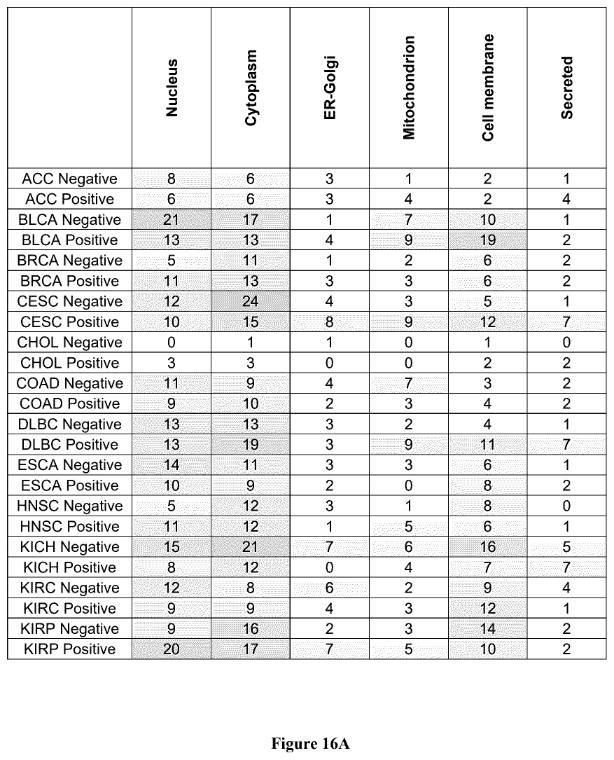

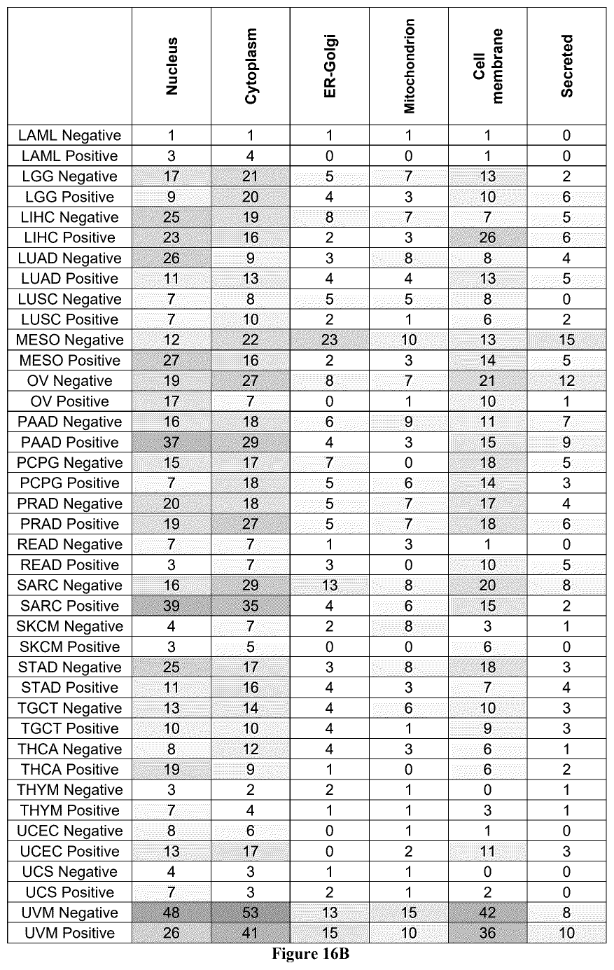

Another aspect of the correlations between tRFs and mRNAs was further examined herein, namely the cellular localization of the protein products whose mRNAs are correlated or anti-correlated with tRFs from tRNA^{HisGTG}. Using information from the UniProt database, six possible destinations were distinguished: nucleus, cytoplasm, endoplasmic reticulum or Golgi, mitochondrion, cell membrane, and secreted. FIG. 22 shows the sub-cellular localization and distribution of the protein products of the mRNAs that are correlated (suffix “Positive”) or anti-correlated (suffix “Negative”) with tRFs from tRNA^{HisGTG}. In FIGS. 16A-16B, each cell lists the number of proteins that localize to the compartment/destination indicated by the corresponding column's header and whose mRNAs are correlated or anti-correlated with tRFs from tRNA^{HisGTG}.

Based on this table, several observations stood out. For example, tRFs from tRNA^{HisGTG} were both positively and negatively correlated to mRNAs whose protein products localize largely to the nucleus, the cytoplasm or the cell membrane. In some instances, these tRFs were correlated/anti-correlated with mRNAs that were secreted from the cell, e.g. MESO, OV and UVM. Also, even though a similarity can be seen in the trends, the range of these correlations diffes from one cancer to the next. For example, in the two melanomas, SKCM and UVM, tRFs from tRNA^{HisGTG} were associated, positively and negatively, with distinctly different numbers of proteins. Another example can be drawn by comparing the two lung cancers, LUAD and LUSC. Evidence from public Ago HITS CLIP-seq data indicates Ago loading of tRFs from tRNA^{HisGTG}, which in turn suggests that some of the negative correlations shown in this figure could result from direct molecular interactions. Independent of whether the relationships captured by FIGS. 16A-16B represent direct or indirect molecular interactions, the present findings link the tRFs from tRNA^{HisGTG} in complex relationships with mRNAs.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of identifying a subject in need of therapeutic intervention to treat and/or prevent a disease, condition, disease recurrence or disease progression, the method comprising characterizing at least one tRNA^{HisGTG} fragment and its relative abundance isolated from a sample obtained from the subject to identify a signature, wherein, when the signature is indicative of a diagnosis of the disease, condition, disease recurrence or disease progression, treatment of the subject is recommended.

2. The method of claim 1, wherein the tRNA^{HisGTG} fragment is at least one selected from the group consisting of a 5′-tRNA fragment (5′-tRF), an internal tRNA fragment (i-tRF), a 3′-tRNA fragment (3′-tRF), a 5′-tRNA half, and a 3′-tRNA half.

3. (canceled)

4. The method of claim 1, wherein the tRNA^{HisGTG} fragment has a length in the range of about 15 nucleotides to about 80 nucleotides.

5. The method of claim 1, wherein the nucleic acid sequence of the tRNA^{HisGTG} fragment comprises at least one selected from the group consisting of SEQ ID NOs: 1-858.

6. The method of claim 1, wherein the tRNA^{HisGTG} fragment is post-transcriptionally modified with at least one selected from the group consisting of guanylation, uridylation, adenylation, P, cP, OH, and aa.

7. The method of claim 6, wherein the post-transcriptionally modified tRNA^{HisGTG} fragment interacts with Argonaute (Ago).

8. The method of claim 1, wherein the relative abundance of the tRNA^{HisGTG} fragment is measured as a ratio of the tRNA^{HisGTG} fragment and at least one other RNA transcript of interest, optionally wherein the at least one other RNA transcript of interest is at least one other tRNA^{HisGTG} fragment that differs by a single nucleotide.

9. The method of claim 1, wherein the tRNA^{HisGTG} fragment is at least one selected from the group consisting of a 5′-tRNA fragment (5′-tRF), an internal-tRNA fragment (i-tRF) and a 3′-tRNA fragment (3′-tRF), and wherein the relative abundance is high in a hormone dependent cancer.

10. (canceled)

11. The method of claim 1, wherein the sample is isolated from a cell, tissue or body fluid obtained from the subject.

12. The method of claim 11, wherein the body fluid is at least one selected from the group consisting of amniotic fluid, aqueous humour and vitreous humour, bile, blood serum, breast milk, cerebrospinal fluid, cerumen, chyle, chyme, endolymph and perilymph, exudates, feces, female ejaculate, gastric acid, gastric juice, lymph, mucus, pericardial fluid, peritoneal fluid, pleural fluid, pus, rheum, saliva, sebum, serous fluid, semen, smegma, sputum, synovial fluid, sweat, tears, urine, vaginal secretion, and vomit.

13. The method of claim 1, wherein the sample is at least one selected from the group consisting of a peripheral blood cell, a tumor cell, a circulating tumor cell, an exosome, a bone marrow cell, a breast cell, a lung cell, a pancreatic cell, a prostate cell, a brain cell, a liver cell, and a skin cell.

14. A method of diagnosing, identifying or monitoring a disease or condition in a subject in need thereof, the method comprising:
hybridizing at least one tRNA^{HisGTG} fragment obtained from a cell obtained from the subject to a panel of oligonucleotides engineered to detect the tRNA^{HisGTG} fragment;
analyzing levels of the tRNA^{HisGTG} fragment present in the cell; wherein a differential in the measured tRNA^{HisGTG} fragment levels compared to a reference is indicative of a diagnosis or identification of breast cancer in the subject; and
providing a treatment regimen to the subject dependent on the differential in the measured tRNA^{HisGTG} fragment levels to the reference.

15. The method of claim 14, wherein the disease or condition is a cancer selected from the group consisting of breast cancer, lung cancer, pancreatic cancer, prostate cancer, liver cancer and eye cancer.

16. The method of claim 14, wherein the disease or condition is a neurological disease selected from the group consisting of Alzheimer's disease, Parkinson's disease and amyotrophic lateral sclerosis.

17. A set of engineered oligonucleotides comprising a mixture of oligonucleotides that are about 15 to about 50 nucleotides in length and capable of hybridizing at least one tRNA^{HisGTG} fragment.

18. The set of claim 17, wherein the nucleic acid sequence of the at least one tRNA^{HisGTG} fragment comprises at least one selected from the group consisting of SEQ ID NOs: 1-858.

19. A kit for high-throughput analysis of tRNA^{HisGTG} fragment in a sample comprising the set of engineered oligonucleotides of claim 17; hybridization reagents; and tRNA fragment isolation reagents.

20. A method of identifying a cell's tissue of origin to treat and/or prevent a disease or condition, disease recurrence, or disease progression in a subject in need thereof, the method comprising:
characterizing the identity of at least one tRNA^{HisGTG} fragment and its relative abundance isolated from a cell obtained from the subject to identify a signature, wherein the signature is indicative of the cell's tissue of origin; and
providing a treatment regimen to the subject dependent on the cell's tissue of origin.

21. The method of claim 20, wherein the nucleic acid sequence of the at least one tRNA^{HisGTG} fragment comprises at least one selected from the group consisting of SEQ ID NOs: 1-858.

22. The method of claim 1, wherein the subject is a human.
